(19) United States
(12) Patent Application Publication
(10) Pub. No.: US 2011/0082323 A1
(43) Pub. Date: Apr. 7, 2011

(54) OLIGOMERIZATION OF OLEFIN WAXES USING METALLOCENE-BASED CATALYST SYSTEMS

(75) Inventors: Brooke L. Small, Kingwood, TX (US); Orson L. Sydora, Houston, TX (US); Albert P. Masino, Tulsa, OK (US); Hu Yang, Manvel, TX (US); Qing Yang, Bartlesville, OK (US); Max P. McDaniel, Bartlesville, OK (US); Eduardo J. Baralt, Kingwood, TX (US); William B. Beaulieu, Tulsa, OK (US)

(73) Assignee: CHEVRON PHILLIPS CHEMICAL COMPANY LP, The Woodlands, TX (US)

(21) Appl. No.: 12/898,465

(22) Filed: Oct. 5, 2010

Related U.S. Application Data
(60) Provisional application No. 61/249,113, filed on Oct. 6, 2009.

(51) Int. Cl. C07C 2/08; C07C 2/02; C07C 11/02

(52) U.S. Cl. 585/18; 585/520; 585/533

(57) ABSTRACT

This disclosure provides for olefin wax oligomer compositions, methods of producing olefin wax oligomer composition, and methods for oligomerizing olefin waxes. This disclosure encompasses metallocene-based olefin wax oligomerization catalyst systems, including those that include a metallocene and an aluminoxane, a metallocene and a solid oxide chemically-treated with an electron withdrawing anion, and a metallocene, a solid oxide chemically-treated with an electron withdrawing anion, and an organoaluminum compound. The olefin wax oligomers prepared with these catalyst systems can decreased needle penetrations, increased viscosity, and an increased drop melt, making them useful as an additive in candles, stone polishes, liquid polishes, and mold release formulations.

### CROSS REFERENCE TO RELATED APPLICATIONS

This application claims priority to and the benefit of U.S. Provisional Patent Application No. 61/249,113, filed Oct. 6, 2009, which is incorporated herein by reference in its entirety.

### TECHNICAL FIELD OF THE INVENTION

This disclosure relates to the metallocene catalyzed oligomerization of olefin waxes to form olefin wax oligomer compositions.

### BACKGROUND OF THE INVENTION

Mono-1-olefins (alpha-olefins), including ethylene, can be oligomerized with catalyst systems employing titanium, zirconium, vanadium, chromium or other metals impregnated on a variety of support materials, often in the presence of activators. These catalyst systems may be useful for both the homooligomerization of ethylene, cooligomerization of ethylene with comonomers such as propylene, 1-butene, 1-hexene, or higher alpha-olefins, or in the homooligomerization olefin having more than 2 carbon atoms. Because of the importance of preparing functional materials, there exists a need and a constant search to develop new olefin polymerization catalysts, catalyst activation processes, and methods of making and using catalysts that will provide enhanced catalytic activities, selectivities, or new oligomeric materials tailored to specific end uses.

One type of transition metal-based catalyst system utilizes metallocene compounds contacted with an activator such as methyl aluminoxane (MAO) to form an oligomerization catalyst system. There remain important challenges in developing catalysts and catalyst systems to produce olefin wax oligomers having desired properties that can be tailored or maintained within a desired specification range.

### SUMMARY OF THE INVENTION

This disclosure provides for olefin wax oligomer compositions, methods of producing olefin wax oligomer composition, and methods for oligomerizing olefin waxes.

The olefin wax oligomer composition comprises an olefin wax oligomer and olefin wax monomers. The olefin wax compositions have a decreased needle penetration, an increased kinematic viscosity, and/or increased drop melt point. In an embodiment, the olefin wax oligomer has a 25° C. needle penetration at least 5 percent lower than the needle penetration of the olefin wax monomer. In an embodiment, the olefin wax oligomer composition has 100° C. kinematic viscosity at least 20 percent higher than the olefin wax monomer. In an embodiment, the olefin wax oligomer composition has a drop melt point, in ° C., at least 5 percent higher than the olefin wax monomer.

The disclosure further provides a method for oligomerizing an olefin wax comprising: a) contacting an olefin wax and a catalyst system; and b) oligomerizing the olefin wax under oligomerization conditions. In an embodiment, the catalyst system comprises a metallocene. In some embodiments, the catalyst system comprises a metallocene and an aluminoxane. In other embodiments, the catalyst system comprises a metallocene, a chemically-treated solid oxide and an organoaluminum compound.

A wide range of metallocenes may be utilized in the catalyst systems for oligomerizing an olefin wax. One exemplary metallocene which may be utilized in the catalyst system is a metallocene having the formula ZrR¹⁰R¹¹X⁹₂ wherein each X⁹ independently is a halogen atom, R¹⁰ and R¹¹ are substituted or unsubstituted η⁵-indenyl groups, and optionally R¹⁰ and R¹¹ may be connected by a linking group. A second exemplary metallocene which may be utilized in the catalyst system is a metallocene having the formula ZrR¹⁰R¹¹X⁹₂ wherein each X⁹ independently is a halogen atom, R¹⁰ is a substituted or unsubstituted η⁵-cyclopentadienyl group, R¹¹ is a substituted or unsubstituted η⁵-fluorenyl group and R¹⁰ and R¹¹ are connected by a linking group. A third exemplary metallocene which may be utilized in the catalyst system is a metallocene having the formula ZrR¹²R¹³R¹⁴X⁹₂ wherein each X⁹ independently is a halogen atom, R¹² is a neutral ether group, R¹³ is a η¹-aminyl group, R¹⁴ is a substituted or unsubstituted η¹-fluorenyl group, and wherein R¹³ and R¹⁴ are connected by a linking group.

### DETAILED DESCRIPTION OF THE INVENTION

### General Description

This disclosure provides for olefin wax oligomer compositions, methods of making the olefin wax oligomer compositions, catalyst systems, and methods making catalyst systems. In particular, this disclosure encompasses oligomerizing one or more olefin waxes using a catalyst system that comprises a metallocene. The catalyst system can further comprise one or more activators. For example, an aluminoxane is one type of activator the can be useful in the catalyst systems and methods described herein. Another type of activator that can be particularly useful is a solid oxide that has been chemically-treated with an electron withdrawing anion, which is fully described herein. This chemically-treated solid oxide (CTSO) also may be referred to throughout this disclosure as a solid super acid (SSA), and these terms are used interchangeably. Other activators can be used with the metallocenes in the catalyst system, either alone, in combination these activators, or in any combination with at least one other activator.

The metallocene-based olefin wax oligomerizations result in olefin wax compositions with particularly useful properties. The produced olefin wax compositions can be useful as an additive in candles, stone polishes, liquid polishes, and mold release formulations.

### DEFINITIONS

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2^{nd} Ed (1997) can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Regarding claim transitional terms or phrases, the transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention. A “consisting essentially of” claim occupies a middle ground between closed claims that are written in a “consisting of” format and fully open claims that are drafted in a “comprising” format. Absent an indication to the contrary, when describing a compound or composition “consisting essentially of” is not to be construed as “comprising,” but is intended to describe the recited component that includes materials which do not significantly alter composition or method to which the term is applied. For example, a feedstock consisting essentially of a material A can include impurities typically present in a commercially produced or commercially available sample of the recited compound or composition. When a claim includes different features and/or feature classes (for example, a method step, feedstock features, and/or product features, among other possibilities), the transitional terms comprising, consisting essentially of, and consisting of, apply only to feature class to which it is utilized and it is possible to have different transitional terms or phrases utilized with different features within a claim. For example a method can comprise several recited steps (and other non-recited steps) but utilize a catalyst system preparation consisting of specific steps but utilize a catalyst system comprising recited components and other non-recited components. While compositions and methods are described in terms of “comprising” various components or steps, the compositions and methods can also “consist essentially of” or “consist of” the various components or steps.

The terms “a,” “an,” and “the” are intended, unless specifically indicated otherwise, to include plural alternatives, e.g., at least one. For instance, the disclosure of “a metallocene” is meant to encompass one metallocene, or mixtures or combinations of more than one metallocene unless otherwise specified.

Groups of elements of the table are indicated using the numbering scheme indicated in the version of the periodic table of elements published in Chemical and Engineering News, 63(5), 27, 1985. In some instances a group of elements may be indicated using a common name assigned to the group; for example alkali metals for Group 1 elements, alkaline earth metals for Group 2 elements, transition metals for Group 3-12 elements, and halogens or halides for Group 17 elements.

For any particular compound disclosed herein, the general structure or name presented is also intended to encompass all structural isomers, conformational isomers, and stereoisomers that can arise from a particular set of substituents, unless indicated otherwise. Thus, a general reference to a compound includes all structural isomers unless explicitly indicated otherwise; e.g. a general reference to pentane includes n-pentane, 2-methyl-butane, and 2,2-dimethylpropane and a general reference to a butyl group includes a n-butyl group, a sec-butyl group, an iso-butyl group, and a t-butyl group. Additionally, the reference to a general structure or name encompasses all enantiomers, diastereomer, and other optical isomers whether in enantiomeric or racemic forms, as well as mixtures of stereoisomers, as the context permits or requires. For any particular formula or name that is presented, any general formula or name presented also encompasses all conformational isomers, regioisomers, and stereoisomers that can arise from a particular set of substituents.

In one aspect, a chemical “group” can be defined or described according to how that group is formally derived from a reference or “parent” compound, for example, by the number of hydrogen atoms that are formally removed from the parent compound to generate the group, even if that group is not literally synthesized in this manner. These groups can be utilized as substituents or coordinated or bonded to metal atoms. By way of example, an “alkyl group” formally can be derived by removing one hydrogen atom from an alkane, while an “alkylene group” formally can be derived by removing two hydrogen atoms from an alkane. Moreover, a more general term can be used to encompass a variety of groups that formally are derived by removing any number (“one or more”) hydrogen atoms from a parent compound, which in this example can be described as an “alkane group,” and which encompasses an “alkyl group,” an “alkylene group,” and materials having three or more hydrogens atoms, as necessary for the situation, removed from an alkane. Throughout, the disclosure that a substituent, ligand, or other chemical moiety can constitute a particular “group” implies that the well-known rules of chemical structure and bonding are followed when that group is employed as described. By way of example, if a metallocene compound having the formula (eta-5-C₅H₅)₂Zr(CH₃)(X) is described, and it is disclosed that X can be an “alkyl group,” an “alkylene group,” or an “alkane group,” the normal rules of valence and bonding are followed. When describing a group as being “derived by,” “derived from,” “formed by,” or “formed from,” such terms are used in a formal sense and are not intended to reflect any specific synthetic methods or procedure, unless specified otherwise or the context requires otherwise. The bonding nomenclature “eta-5” is also written “η⁵-” throughout.

Many groups are specified according to the atom that is bonded to the metal or bonded to another chemical moiety as a substituent, such as an “oxygen-bonded group,” which is also called an “oxygen group.” For example, an oxygen-bonded group includes species such as hydrocarboxy (—OR where R is a hydrocarbyl group), alkoxide (—OR where R is an alkyl group), aryloxide (—OAr where Ar is an aryl group), or substituted analogs thereof, which function as ligands or substituents in the specified location. Also, unless otherwise specified, any carbon-containing group for which the number of carbon atoms is not specified can have, according to proper chemical practice, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms, or any range or combination of ranges between these values. For example, unless otherwise specified, any carbon-containing group can have from 1 to 30 carbon atoms, from 1 to 25 carbon atoms, from 1 to 20 carbon atoms, from 1 to 15 carbon atoms, from 1 to 10 carbon atoms, or from 1 to 5 carbon atoms, and the like. Moreover, other identifiers or qualifying terms may be utilized to indicate the presence or absence of a particular substituent, a particular regiochemistry and/or stereochemistry, or the presence of absence of a branched underlying structure or backbone.

The term “substituted” when used to describe a group, for example, when referring to a substituted analog of a particular group, is intended to describe any non-hydrogen moiety that formally replaces a hydrogen in that group, and is intended to be non-limiting. A group or groups can also be referred to herein as “unsubstituted” or by equivalent terms such as “non-substituted,” which refers to the original group in which a non-hydrogen moiety does not replace a hydrogen within that group. “Substituted” is intended to be non-limiting and include inorganic substituents or organic substituents as understood by one of ordinary skill in the art.

The term “organyl group” is used herein in accordance with the definition specified by IUPAC: an organic substituent group, regardless of functional type, having one free valence at a carbon atom. Similarly, an “organylene group” refers to an organic group, regardless of functional type, derived by removing two hydrogen atoms from an organic compound, either two hydrogen atoms from one carbon atom or one hydrogen atom from each of two different carbon atoms. An “organic group” refers to a generalized group formed by removing one or more hydrogen atoms from carbon atoms of an organic compound. Thus, an “organyl group,” an “organylene group,” and an “organic group” can contain organic functional group(s) and/or atom(s) other than carbon and hydrogen, that is, an organic group that can comprise functional groups and/or atoms in addition to carbon and hydrogen. For instance, non-limiting examples of atoms other than carbon and hydrogen include halogens, oxygen, nitrogen, phosphorus, and the like. Non-limiting examples of functional groups include ethers, aldehydes, ketones, esters, sulfides, amines, and phosphines, and so forth. In one aspect, the hydrogen atom(s) removed to form the “organyl group,” “organylene group,” or “organic group” can be attached to a carbon atom belonging to a functional group, for example, an acyl group (—C(O)R), a formyl group (—C(O)H), a carboxy group (—C(O)OH), a hydrocarboxycarbonyl group (—C(O)OR), a cyano group (—C≡N), a carbamoyl group (—C(O)NH₂), a N-hydrocarbylcarbamoyl group (—C(O)NHR), or N,N′-dihydrocarbylcarbamoyl group (—C(O)NR₂), among other possibilities. In another aspect, the hydrogen atom(s) removed to form the “organyl group,” “organylene group,” or “organic group” can be attached to a carbon atom not belonging to, and remote from, a functional group, for example, —CH₂C(O)CH₃, —CH₂NR₂, and the like. An “organyl group,” “organylene group,” or “organic group” can be aliphatic, inclusive of being cyclic or acyclic, or can be aromatic, and/or linear or branched. “Organyl groups,” “organylene groups,” and “organic groups” also encompass heteroatom-containing rings, heteroatom-containing ring systems, heteroaromatic rings, and heteroaromatic ring systems. “Organyl groups,” “organylene groups,” and “organic groups” can be linear or branched unless otherwise specified. Finally, it is noted that the “organyl group,” “organylene group,” or “organic group” definitions include “hydrocarbyl group,” “hydrocarbylene group,” “hydrocarbon group,” respectively, and “alkyl group,” “alkylene group,” and “alkane group,” respectively, (among others known to those having ordinary skill in the art) as members. When bonded to a transition metal, an “organyl group,” “organylene group,” or “organic group” can be further described according to the usual η^{x} (eta-x) nomenclature, in which x is an integer corresponding to the number of atoms which are coordinated to the transition metal or are expected to be coordinated to the transition metal, for example, according to the 18-electron rule.

The term “hydrocarbon” whenever used in this specification and claims refers to a compound containing only carbon and hydrogen. Other identifiers may be utilized to indicate the presence of particular groups in the hydrocarbon (e.g. halogenated hydrocarbon indicates that the presence of one or more halogen atoms replacing an equivalent number of hydrogen atoms in the hydrocarbon). The term “hydrocarbyl group” is used herein in accordance with the definition specified by IUPAC: a univalent group formed by removing a hydrogen atom from a hydrocarbon (that is, a group containing only carbon and hydrogen). Non-limiting examples of hydrocarbyl groups include ethyl, phenyl, tolyl, propenyl, and the like. Similarly, a “hydrocarbylene group” refers to a group formed by removing two hydrogen atoms from a hydrocarbon, either two hydrogen atoms from one carbon atom or one hydrogen atom from each of two different carbon atoms. Therefore, in accordance with the terminology used herein, a “hydrocarbon group” refers to a generalized group formed by removing one or more hydrogen atoms (as necessary for the particular group) from a hydrocarbon. A “hydrocarbyl group,” “hydrocarbylene group,” and “hydrocarbon group” can be aliphatic or aromatic, acyclic or cyclic groups, and/or linear or branched. A “hydrocarbyl group,” “hydrocarbylene group,” and “hydrocarbon group” can include rings, ring systems, aromatic rings, and aromatic ring systems, which contain only carbon and hydrogen. When bonded to a transition metal, a “hydrocarbyl group,” “hydrocarbylene group,” and “hydrocarbon group” can be further described according to the usual η^{x} (eta-x) nomenclature, in which x is an integer corresponding to the number of atoms which are coordinated to the transition metal or are expected to be coordinated to the transition metal, for example, according to the 18-electron rule. “Hydrocarbyl groups,” “hydrocarbylene groups,” and “hydrocarbon groups” include, by way of example, aryl, arylene, arene groups, alkyl, alkylene, alkane group, cycloalkyl, cycloalkylene, cycloalkane groups, aralkyl, aralkylene, and aralkane groups, respectively, among other groups as members.

An aliphatic compound is a class of acyclic or cyclic, saturated or unsaturated, and/or linear or branched carbon compounds that excludes aromatic compounds. An “aliphatic group” is a generalized group formed by removing one or more hydrogen atoms (as necessary for the particular group) from carbon atom of an aliphatic compound. That is, an aliphatic compound is a non-aromatic organic compound. Aliphatic compounds and therefore aliphatic groups can contain organic functional group(s) and/or atom(s) other than carbon and hydrogen.

The term “alkane” whenever used in this specification and claims refers to a saturated hydrocarbon compound. Other identifiers may be utilized to indicate the presence of particular groups in the alkane (e.g. halogenated alkane indicates that the presence of one or more halogen atoms replacing an equivalent number of hydrogen atoms in the alkane). The term “alkyl group” is used herein in accordance with the definition specified by IUPAC: a univalent group formed by removing a hydrogen atom from an alkane. Similarly, an “alkylene group” refers to a group formed by removing two hydrogen atoms from an alkane (either two hydrogen atoms from one carbon atom or one hydrogen atom from two different carbon atoms). An “alkane group” is a general term that refers to a group formed by removing one or more hydrogen atoms (as necessary for the particular group) from an alkane. An “alkyl group,” “alkylene group,” and “alkane group” can be acyclic or cyclic groups, and/or can be linear or branched unless otherwise specified. A primary, secondary, and tertiary alkyl group are derived by removal of a hydrogen atom from a primary, secondary, tertiary carbon atom, respectively, of an alkane. The n-alkyl group derived by removal of a hydrogen atom from a terminal carbon atom of a linear alkane. The groups RCH₂ (R≠H), R₂CH (R≠H), and R₃C (R≠H) are primary, secondary, and tertiary alkyl groups, respectively.

A cycloalkane is a saturated cyclic hydrocarbon, with or without side chains, for example, cyclobutane. Other identifiers may be utilized to indicate the presence of particular groups in the cycloalkane (e.g. halogenated cycloalkane indicates that the presence of one or more halogen atoms replacing an equivalent number of hydrogen atoms in the cycloalkane). Unsaturated cyclic hydrocarbons having one or more endocyclic double or one triple bond are called cycloalkenes and cycloalkynes, respectively. Those having only one, only two, only three, and so forth, such multiple bond can be identified by use of the term “mono,” “di,” “tri,” and so forth, within the name; e.g. cyclomonoenes, cycloalkadienes, cycloalkatrienes, and so forth. Other identifiers may be utilized to indicate the position of the multiple bonds and/or the presence of particular groups in the cycloalkenes or cycloalkynes.

A “cycloalkyl group” is a univalent group derived by removing a hydrogen atom from a ring carbon atom from a cycloalkane. For example, a 1-methylcyclopropyl group and a 2-methylcyclopropyl group are illustrated as follows.

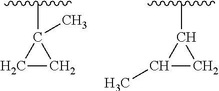

Similarly, a “cycloalkylene group” refers to a group derived by removing two hydrogen atoms from a cycloalkane, at least one of which is a ring carbon. Thus, a “cycloalkylene group” includes both a group derived from a cycloalkane in which two hydrogen atoms are formally removed from the same ring carbon, a group derived from a cycloalkane in which two hydrogen atoms are formally removed from two different ring carbons, and a group derived from a cycloalkane in which a first hydrogen atom is formally removed from a ring carbon and a second hydrogen atom is formally removed from a carbon atom that is not a ring carbon. An “cycloalkane group” refers to a generalized group formed by removing one or more hydrogen atoms (as necessary for the particular group and at least one of which is a ring carbon) from a cycloalkane.

The term “alkene” whenever used in this specification and claims refers a linear or branched hydrocarbon olefin that has one or more carbon-carbon double bonds. Alkenes having only one, only two, only three, and so forth, such multiple bond can be identified by use of the term “mono,” “di,” “tri,” and the like, within the name. For example, alkamonoenes, alkadienes, and alkatrienes refer to a linear or branched hydrocarbon olefin having only one carbon-carbon double bond (general formula CₙH₂ₙ), only two carbon-carbon double bonds (general formula CₙH₂ₙ₋₂), and only three carbon-carbon double bonds (general formula CₙH₂ₙ₋₄), respectively. Alkenes, can be further identified by the position of the carbon-carbon double bond(s). Other identifiers may be utilized to indicate the presence or absence of particular groups within an alkene. For example, a haloalkene refers to an alkene having one or more hydrogen atoms replace with a halogen atom.

An “alkenyl group” is a univalent group derived from an alkene by removal of a hydrogen atom from any carbon atom of the alkene. Thus, “alkenyl group” includes groups in which the hydrogen atom is formally removed from an sp² hybridized (olefinic) carbon atom and groups in which the hydrogen atom is formally removed from any other carbon atom. For example and unless otherwise specified, 1-propenyl (—CH═CHCH₃), 2-propenyl [(CH₃)C═CH₂], and 3-propenyl (—CH₂CH═CH₂) groups are encompassed with the term “alkenyl group.” Similarly, an “alkenylene group” refers to a group formed by formally removing two hydrogen atoms from an alkene, either two hydrogen atoms from one carbon atom or one hydrogen atom from two different carbon atoms. An “alkene group” refers to a generalized group formed by removing one or more hydrogen atoms (as necessary for the particular group) from an alkene. When the hydrogen atom is removed from a carbon atom participating in a carbon-carbon double bond, the regiochemistry of the carbon from which the hydrogen atom is removed, and regiochemistry of the carbon-carbon double bond can both be specified. Other identifiers may be utilized to indicate the presence or absence of particular groups within an alkene group. Alkene groups can also be further identified by the position of the carbon-carbon double bond.

The term “alkyne” whenever used in this specification and claims refers to a linear or branched hydrocarbon olefin that has one or more carbon-carbon triple bonds and the general formula CₙH₂ₙ₋₂. Alkynes having only one, only two, only three, and the like, such multiple bond can be identified by use of the term “mono,” “di,” “tri,” and so forth, within the name. For example, alkamonoynes, alkadiynes, and alkatriynes refer to a hydrocarbon olefin having only one carbon-carbon triple bond (general formula CₙH₂ₙ₋₂), only two carbon-carbon triple bonds (general formula CₙH₂ₙ₋₆), and only three carbon-carbon triple bonds (general formula CₙH₂ₙ₋₁₀), respectively. Alkynes, alkadiynes, and alkatriynes can be further identified by the position of the carbon-carbon triple bond(s). Other identifiers may be utilized to indicate the presence or absence of particular groups within an alkyne. For example, a haloalkyne refers to an alkyne having one or more hydrogen atoms replace with a halogen atom.

An “alkynyl group” is a univalent group derived from an alkyne by removal of a hydrogen atom from any carbon atom of the alkyne. Thus, “alkynyl group” includes groups in which the hydrogen atom is formally removed from an sp hybridized (acetylenic) carbon atom and groups in which the hydrogen atom is formally removed from any other carbon atom. For example and unless otherwise specified, 1-propynyl (—C≡CCH₃) and 3-propynyl (HC≡CCH₂—) groups are all encompassed with the term “alkynyl group.” Similarly, an “alkynylene group” refers to a group formed by formally removing two hydrogen atoms from an alkyne, either two hydrogen atoms from one carbon atom if possible or one hydrogen atom from two different carbon atoms. An “alkyne group” refers to a generalized group formed by removing one or more hydrogen atoms (as necessary for the particular group) from an alkyne. Other identifiers may be utilized to indicate the presence or absence of particular groups within an alkyne group. Alkyne groups can also be further identified by the position of the carbon-carbon triple bond.

The term “olefin” whenever used in this specification and claims refers to compound that has at least one carbon-carbon double bond that is not part of an aromatic ring or ring system. The term “olefin” includes aliphatic, aromatic, cyclic or acyclic, and/or linear and branched compounds having at least one carbon-carbon double bond that is not part of an aromatic ring or ring system unless specifically stated, otherwise. The term “olefin,” by itself, does not indicate the presence or absence of heteroatoms and/or the presence or absence of other carbon-carbon double bonds unless explicitly indicated. Olefins can also be further identified by the position of the carbon-carbon double bond. It is noted that alkenes, alkamonoenes, alkadienes, alkatrienes, cycloalkenes, cycloalkadienes, are members of the class of olefins. The olefin can be further identified by the position of the carbon-carbon double bond(s).

The term “alpha olefin” as used in this specification and claims refers to an olefin that has a double bond between the first and second carbon atom of a contiguous chain of carbon atoms. The term “alpha olefin” includes linear and branched alpha olefins unless expressly stated otherwise. In the case of branched alpha olefins, a branch may be at the 2-position (a vinylidene) and/or the 3-position or higher with respect to the olefin double bond. The term “vinylidene” whenever used in this specification and claims refers to an alpha olefin having a branch at the 2-position with respect to the olefin double bond. By itself, the term “alpha olefin” does not indicate the presence or absence of heteroatoms and/or the presence or absence of other carbon-carbon double bonds unless explicitly indicated. The terms “hydrocarbon alpha olefin” or “alpha olefin hydrocarbon” refer to alpha olefin compounds containing only hydrogen and carbon.

The term “linear alpha olefin” as used herein refers to a linear olefin having a double bond between the first and second carbon atom. The term “linear alpha olefin” by itself does not indicate the presence or absence of heteroatoms and/or the presence or absence of other carbon-carbon double bonds, unless explicitly indicated. The terms “linear hydrocarbon alpha olefin” or “linear alpha olefin hydrocarbon” refers to linear alpha olefin compounds containing only hydrogen and carbon.

The term “normal alpha olefin” whenever used in this specification and claims refers to a linear hydrocarbon mono-olefin having a double bond between the first and second carbon atom. It is noted that “normal alpha olefin” is not synonymous with “linear alpha olefin” as the term “linear alpha olefin” can include linear olefinic compounds having a double bond between the first and second carbon atoms and having heteroatoms and/or additional double bonds.

The term “consists essentially of normal alpha olefin(s)” or variations thereof are used in the specification and claims to refer to commercially available normal alpha olefin product(s). The commercially available normal alpha olefin product can contain non-normal alpha olefin impurities such as vinylidenes, internal olefins, branched alpha olefins, paraffins, and diolefins, among other impurities, which are not removed during the normal alpha olefin production process. One of ordinary skill in the art will recognize that the identity and quantity of the specific impurities present in the commercial normal alpha olefin product will depend upon the source of commercial normal alpha olefin product. Additionally, when applied to a normal alpha olefin of a single carbon number, the term “consists essentially of a normal alpha olefin(s)” also includes small quantities (e.g. less than 5, 4, 3, 2, or 1 weight %) of olefins having a different carbon number than the recited normal alpha olefin carbon number which are not removed during the production of the single carbon number normal alpha olefin production process. Consequently, the term “consists essentially of normal alpha olefins” and its variants is not intended to limit the amount/quantity of the non-linear alpha olefin components (or in relation to carbon number the amount of a non-recited carbon number) any more stringently than the amounts/quantities present in a particular commercial normal alpha olefin product, unless explicitly stated. One source of commercially available alpha olefins products are those produced by the oligomerization of ethylene. A second source of commercially available alpha olefin products are those which are produced, and optionally isolated from, Fischer-Tropsch synthesis streams. One source of commercially available normal alpha olefin products produced by ethylene oligomerization which can be utilized as an olefin feedstock is Chevron Phillips Chemical Company LP, The Woodlands, Tex., USA. Other sources of commercially available normal alpha olefin products produced by ethylene oligomerization which can be utilized as an olefin feedstock include Inneos Oligomers (Feluy, Belgium), Shell Chemicals Corporation (Houston, Tex., USA or London, United Kingdom), Idemitsu Kosan (Tokyo, Japan), and Mitsubishi Chemical Corporation (Tokyo, Japan), among others. One source of commercially available normal alpha olefin products produced, and optionally isolated from Fisher-Tropsch synthesis streams includes Sasol (Johannesburg, South Africa), among others.

An “aromatic group” refers to a generalized group formed by removing one or more hydrogen atoms (as necessary for the particular group and at least one of which is an aromatic ring carbon atom) from an aromatic compound. Thus, an “aromatic group” as used herein refers to a group derived by removing one or more hydrogen atoms from an aromatic compound, that is, a compound containing a cyclically conjugated hydrocarbon that follows the Hückel (4n+2) rule and containing (4n+2) pi-electrons, where n is an integer from 1 to about 5. Aromatic compounds and hence “aromatic groups” can be monocyclic or polycyclic unless otherwise specified. Aromatic compounds include “arenes” (hydrocarbon aromatic compounds) and “heteroarenes,” also termed “hetarenes” (heteroaromatic compounds formally derived from arenes by replacement of one or more methine (—C═) carbon atoms by trivalent or divalent heteroatoms, in such a way as to maintain the continuous pi-electron system characteristic of aromatic systems and a number of out-of-plane pi-electrons corresponding to the Hückel rule (4n+2)). While arene compounds and heteroarene compounds are mutually exclusive members of the group of aromatic compounds, a compound that has both an arene group and a heteroarene group is considered a heteroarene compound. Aromatic compounds, arenes, and heteroarenes can be mono- or polycyclic unless otherwise specified. Examples of arenes include, but are not limited to, benzene, naphthalene, and toluene, among others. Examples of heteroarenes include, but are not limited to furan, pyridine, and methylpyridine, among others. When bonded to a transition metal, an aromatic group can be further described according to the usual η^{x} (eta-x) nomenclature, in which x is an integer corresponding to the number of atoms which are coordinated to the transition metal or are expected to be coordinated to the transition metal, for example, according to the 18-electron rule. As disclosed herein, the term “substituted” can be used to describe an aromatic group wherein any non-hydrogen moiety formally replaces a hydrogen in that group, and is intended to be non-limiting.

An “aryl group” is a group derived from the formal removal of a hydrogen atom from an aromatic hydrocarbon ring carbon atom from an arene compound. One example of an “aryl group” is ortho-tolyl (o-tolyl), the structure of which is shown here.

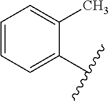

Similarly, an “arylene group” refers to a group formed by removing two hydrogen atoms (at least one of which is from an aromatic hydrocarbon ring carbon) from an arene. An “arene group” refers to a generalized group formed by removing one or more hydrogen atoms (as necessary for the particular group and at least one of which is an aromatic hydrocarbon ring carbon) from an arene. However, if a group contains both arene and heteroarene moieties its classification depends upon the particular moiety from which the hydrogen atom was removed, that is, an arene group if the removed hydrogen came from a carbon atom of an aromatic hydrocarbon ring or ring system and a heteroarene group if the removed hydrogen came from a carbon atom of a heteroaromatic ring or ring system. When bonded to a transition metal, an “aryl group,” “arylene group,” and “arene group” can be further described according to the usual η^{x} (eta-x) nomenclature, in which x is an integer corresponding to the number of atoms which are coordinated to the transition metal or are expected to be coordinated to the transition metal, for example, according to the 18-electron rule.

A “heterocyclic compound” is a cyclic compound having at least two different elements as ring member atoms. For example, heterocyclic compounds can comprise rings containing carbon and nitrogen (for example, tetrahydropyrrole), carbon and oxygen (for example, tetrahydrofuran), or carbon and sulfur (for example, tetrahydrothiophene), among others. Heterocyclic compounds and heterocyclic groups can be either aliphatic or aromatic. When bonded to a transition metal, a heterocyclic compound can be further described according to the usual η^{x} (eta-x) nomenclature, in which x is an integer corresponding to the number of atoms which are coordinated to the transition metal or are expected to be coordinated to the transition metal, for example, according to the 18-electron rule.

A “heterocyclyl group” is a univalent group formed by removing a hydrogen atom from a heterocyclic ring or ring system carbon atom of a heterocyclic compound. By specifying that the hydrogen atom is removed from a heterocyclic ring or ring system carbon atom, a “heterocyclyl group” is distinguished from a “cycloheteryl group,” in which a hydrogen atom is removed from a heterocyclic ring or ring system heteroatom. For example, a pyrrolidin-2-yl group illustrated below is one example of a “heterocyclyl group,” and a pyrrolidin-1-yl group illustrated below is one example of a “cycloheteryl” group.”

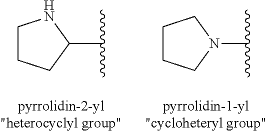

Similarly, a “heterocyclylene group” or more simply, a “heterocyclene group,” refers to a group formed by removing two hydrogen atoms from a heterocyclic compound, at least one of which is from a heterocyclic ring or ring system carbon. Thus, in a “heterocyclylene group,” at least one hydrogen is removed from a heterocyclic ring or ring system carbon atom, and the other hydrogen atom can be removed from any other carbon atom, including for example, the same heterocyclic ring or ring system carbon atom, a different heterocyclic ring or ring system ring carbon atom, or a non-ring carbon atom. A “heterocyclic group” refers to a generalized group formed by removing one or more hydrogen atoms (as necessary for the particular group and at least one of which is a heterocyclic ring carbon atom) from a heterocyclic compound. When bonded to a transition metal, a “heterocyclyl group,” “heterocyclylene group,” and “heterocyclic group” can be further described according to the usual η^{x} (eta-x) nomenclature, in which x is an integer corresponding to the number of atoms which are coordinated to the transition metal or are expected to be coordinated to the transition metal, for example, according to the 18-electron rule.

A “cycloheteryl group” is a univalent group formed by removing a hydrogen atom from a heterocyclic ring or ring system heteroatom of a heterocyclic compound, as illustrated. By specifying that the hydrogen atom is removed from a heterocyclic ring or ring system heteroatom and not from a ring carbon atom, a “cycloheteryl group” is distinguished from a “heterocyclyl group” in which a hydrogen atom is removed from a heterocyclic ring or ring system carbon atom. Similarly, a “cycloheterylene group” refers to a group formed by removing two hydrogen atoms from an heterocyclic compound, at least one of which is removed from a heterocyclic ring or ring system heteroatom of the heterocyclic compound; the other hydrogen atom can be removed from any other atom, including for example, a heterocyclic ring or ring system ring carbon atom, another heterocyclic ring or ring system heteroatom, or a non-ring atom (carbon or heteroatom). A “cyclohetero group” refers to a generalized group formed by removing one or more hydrogen atoms (as necessary for the particular group and at least one of which is from a heterocyclic ring or ring system heteroatom) from a heterocyclic compound. When bonded to a transition metal, a “cycloheteryl group,” “cycloheterylene group,” and “cyclohetero group” can be further described according to the usual η^{x} (eta-x) nomenclature, in which x is an integer corresponding to the number of atoms which are coordinated to the transition metal or are expected to be coordinated to the transition metal, for example, according to the 18-electron rule.

A “heteroaryl group” is a class of “heterocyclyl group” and is a univalent group formed by removing a hydrogen atom from a heteroaromatic ring or ring system carbon atom of a heteroarene compound. By specifying that the hydrogen atom is removed from a ring carbon atom, a “heteroaryl group” is distinguished from an “arylheteryl group,” in which a hydrogen atom is removed from a heteroaromatic ring or ring system heteroatom. For example, an indol-2-yl group illustrated below is one example of a “heteroaryl group,” and an indol-1-yl group illustrated below is one example of an “arylheteryl” group.”

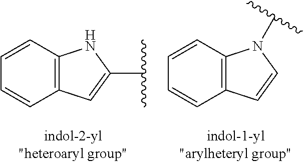

Similarly, a “heteroarylene group” refers to a group formed by removing two hydrogen atoms from a heteroarene compound, at least one of which is from a heteroarene ring or ring system carbon atom. Thus, in a “heteroarylene group,” at least one hydrogen is removed from a heteroarene ring or ring system carbon atom, and the other hydrogen atom can be removed from any other carbon atom, including for example, a heteroarene ring or ring system carbon atom, or a non-heteroarene ring or ring system atom. A “heteroarene group” refers to a generalized group formed by removing one or more hydrogen atoms (as necessary for the particular group and at least one of which is a heteroarene ring or ring system carbon atom) from a heteroarene compound. When bonded to a transition metal, a “heteroaryl group,” “heteroarylene group,” and “heteroarene group” can be further described according to the usual η^{x} (eta-x) nomenclature, in which x is an integer corresponding to the number of atoms which are coordinated to the transition metal or are expected to be coordinated to the transition metal, for example, according to the 18-electron rule.

An “arylheteryl group” is a class of “cycloheteryl group” and is a univalent group formed by removing a hydrogen atom from a heteroaromatic ring or ring system heteroatom of a heteroaryl compound, as illustrated. By specifying that the hydrogen atom is removed from of a heteroaromatic ring or ring system heteroatom and not from a heteroaromatic ring or ring system carbon atom, an “arylheteryl group” is distinguished from a “heteroaryl group” in which a hydrogen atom is removed from a heteroaromatic ring or a ring system carbon atom. Similarly, an “arylheterylene group” refers to a group formed by removing two hydrogen atoms from an heteroaryl compound, at least one of which is removed from a heteroaromatic ring or ring system heteroatom of the heteroaryl compound; the other hydrogen atom can, be removed from any other atom, including for example, a heteroaromatic ring or ring system ring carbon atom, another heteroaromatic ring or ring system heteroatom, or a non-ring atom (carbon or heteroatom) from a heteroaromatic compound. An “arylhetero group” refers to a generalized group formed by removing one or more hydrogen atoms (as necessary for the particular group and at least one of which is from a heteroaromatic ring or ring system) heteroatom from a heteroarene compound. When bonded to a transition metal, an “arylheteryl group,” “arylheterylene group,” and “arylhetero group” can be further described according to the usual η^{x} (eta-x) nomenclature, in which x is an integer corresponding to the number of atoms which are coordinated to the transition metal or are expected to be coordinated to the transition metal, for example, according to the 18-electron rule.

An “organoheteryl group” is a univalent group containing carbon, which are thus organic, but which have their free valence at an atom other than carbon. Thus, organoheteryl and organyl groups are complementary and mutually exclusive. Organoheteryl groups can be cyclic or acyclic, and/or aliphatic or aromatic, and thus encompasses aliphatic “cycloheteryl groups” such as pyrrolidin-1-yl, aromatic “arylheteryl groups” such as indol-1-yl, and acyclic groups such as organylthio, trihydrocarbylsilyl, and aryloxide, among others. Similarly, an “organoheterylene group” is a divalent group containing carbon and at least one heteroatom having two free valences, at least one of which is at a heteroatom. An “organohetero group” is a generalized group containing carbon and at least one heteroatom having one or more free valences (as necessary for the particular group and at least one of which is at a heteroatom) from an organohetero compound. When bonded to a transition metal, an “organoheteryl group,” an “organoheterylene group,” or an “organohetero group” can be further described according to the usual η^{x} (eta-x) nomenclature, in which x is an integer corresponding to the number of atoms which are coordinated to the transition metal or are expected to be coordinated to the transition metal, for example, according to the 18-electron rule.

An “aralkyl group” is an aryl-substituted alkyl group having a free valance at a non-aromatic carbon atom, for example, a benzyl group. Similarly, an “aralkylene group” is an aryl-substituted alkylene group having two free valances at a single non-aromatic carbon atom or a free valence at two non-aromatic carbon atoms while an “aralkane group” is a generalized is an aryl-substituted alkane group having one or more free valances at a non-aromatic carbon atom(s). A “heteroaralkyl group” is a heteroaryl-substituted alkyl group having a free valence at a non-heteroaromatic ring or ring system carbon atom. Similarly a “heteroaralkylene group” is a heteroaryl-substituted alkylene group having a two free valances at a single non-heteroaromatic ring or ring system carbon atom or a free valence at two non-heteroaromatic ring or ring system carbon atoms while a “heteroaralkane group” is a generalized aryl-substituted alkane group having one or more free valances at a non-heteroaromatic ring or ring system carbon atom(s).

A “halide” has its usual meaning. Examples of halides include fluoride, chloride, bromide, and iodide.

An “oxygen group,” also called an “oxygen-bonded group,” is a chemical moiety having at least one free valence on an oxygen atom. Exemplary “oxygen groups” include, but are not limited to, hydroxy (—OH), —OR, —OC(O)R, —OSiR₃, —OPR₂, —OAlR₂, —OSiR₂, —OGeR₃, —OSnR₃, —OSO₂R, —OSO₂OR, —OBR₂, —OB(OR)₂, —OAlR₂, —OGaR₂, —OP(O)R₂, —OAs(O)R₂, —OAlR₂, and the like, including substituted analogs thereof. In one aspect, each R can be independently a hydrocarbyl group; e.g. each R can be independently alkyl, cycloalkyl, aryl, aralkyl, substituted alkyl, substituted cycloalkyl, substituted aryl, or substituted aralkyl. In an “oxygen group” having more than one free valency, the other free valencies can be on atom(s) other than oxygen, for example carbon, in accord with the rules of chemical structure and bonding.

A “sulfur group,” also called a “sulfur-bonded group,” is a chemical moiety having at least one free valence on a sulfur atom. Exemplary “sulfur group(s)” include, but are not limited to, —SH, —SR, —SCN, —S(O)R, —SO₂R, and the like, including substituted analogs thereof. In one aspect, each R can be independently a hydrocarbyl group; e.g. each R can be independently alkyl, cycloalkyl, aryl, aralkyl, substituted alkyl, substituted cycloalkyl, substituted aryl, or substituted aralkyl. In a “sulfur group” having more than one free valency, the other free valencies can be on atom(s) other than sulfur, for example carbon, in accord with the rules of chemical structure and bonding.

A “nitrogen group,” also called a “nitrogen-bonded group,” is a chemical moiety having at least one free valence on a nitrogen atom. Exemplary “nitrogen groups” include, but are not limited to, an aminyl group (—NH₂), an N-substituted aminyl group (—NRH), an N,N-disubstituted aminyl group (—NR₂), a hydrazido group (—NHNH₂), an N¹-substituted hydrazido group (—NRNH₂), an N²-substituted hydrazido group (—NHNRH), an N²,N²-disubstituted hydrazido group (—NHNR₂), a nitro group (—NO₂), an azido group (—N₃), an amidyl group (—NHC(O)R), an N-substituted amido group (—NRC(O)R), and the like, including substituted analogs thereof. In one aspect, each R can be independently a hydrocarbyl group; e.g. each R can be independently alkyl, cycloalkyl, aryl, aralkyl, substituted alkyl, substituted cycloalkyl, substituted aryl, or substituted aralkyl. In a “nitrogen group” having more than one free valency, the other free valencies can be on any atom(s) in the group in accord with the rules of chemical structure and bonding, including atoms other than nitrogen, for example, carbon.

A “phosphorus group,” also called a “phosphorus-bonded group,” is a chemical moiety having at least one free valence on a phosphorus atom. Exemplary “phosphorous groups include, but are not limited to, —PH₂, —PHR, —PR₂, —P(O)R₂, —P(OR)₂, —P(O)(OR)₂, —P(NR₂)₂, —P(O)(NR₂)₂, and the like, including substituted analogs thereof. In one aspect, each R can be independently a hydrocarbyl group; e.g. each R can be independently alkyl, cycloalkyl, aryl, aralkyl, substituted alkyl, substituted cycloalkyl, substituted aryl, or substituted aralkyl. In a “phosphorus group” having more than one free valency, the other free valencies can be on any atom(s) in the group in accord with the rules of chemical structure and bonding, including atoms other than phosphorus, for example, carbon.

An “arsenic group,” also called an “arsenic-bonded group,” is a chemical moiety having a free valence on an arsenic atom. Exemplary “arsenic groups” include, —AsH₂, —AsHR, —AsR₂, —As(O)R₂, —As(OR)₂, —As(O)(OR)₂, and the like, including substituted analogs thereof. In one aspect, each R can be independently a hydrocarbyl group; e.g. each R can be independently alkyl, cycloalkyl, aryl, aralkyl, substituted alkyl, substituted cycloalkyl, substituted aryl, or substituted aralkyl. In an “arsenic group” having more than one free valency, the other free valencies can be on any atom(s) in the group in accord with the rules of chemical structure and bonding, including atoms other than phosphorus, for example, carbon.

A “silicon group,” also called a “silicon-bonded group,” is a generalized chemical moiety having at least one free valence on a silicon atom. A “silyl group” is a chemical moiety having at least one free valence on a silicon atom. Exemplary “silyl groups” include, but are not limited to, —SiH₃, —SiH₂R, —SiHR₂, —SiR₃, —SiR₂OR, —SiR(OR)₂, —Si(OR)₃ and the like. In one aspect, each R can be independently a hydrocarbyl group; e.g. each R can be independently alkyl, cycloalkyl, aryl, aralkyl, substituted alkyl, substituted cycloalkyl, substituted aryl, or substituted aralkyl. In a “silicon group” having more than one free valency, the other free valencies can be on any atom(s) in the group in accord with the rules of chemical structure and bonding, including atoms other than silicon, for example, carbon.

A “germanium group,” also called or a “germanium-bonded group,” is a generalized chemical moiety having at least free valence on a germanium atom. A “germanyl group” is a chemical moiety having at least one free valence on a germanium atom. Exemplary “germanyl groups” include, but are not limited to, —GeH₃, —GeH₂R, —GeHR₂, —GeR₃, —GeR₂OR, —GeR(OR)₂, —Ge(OR)₃ and the like. In one aspect, each R can be independently a hydrocarbyl group; e.g. each R can be independently alkyl, cycloalkyl, aryl, aralkyl, substituted alkyl, substituted cycloalkyl, substituted aryl, or substituted aralkyl. In a “germanium group” having more than one free valency, the other free valencies can be on any atom(s) in the group in accord with the rules of chemical structure and bonding, including atoms other than germanium, for example, carbon.

A “tin group,” also called a “tin-bonded group,” is a generalized chemical moiety having at least one free valence on a tin atom. A “stannyl group” is a chemical moiety having a one free valence on a tin atom. Exemplary “stannyl groups” include, but is not limited to, —SnH₃, —SnH₂R, —SnHR₂, —SnR₃ and —Sn(OR)₃. In one aspect, each R can be independently a hydrocarbyl group; e.g. each R can be independently alkyl, cycloalkyl, aryl, aralkyl, substituted alkyl, substituted cycloalkyl, substituted aryl, or substituted aralkyl. In a “tin group” having more than one free valency, the other free valencies can be on any atom(s) in the group in accord with the rules of chemical structure and bonding, including atoms other than tin, for example, carbon.

A “lead group,” also called a “lead-bonded group,” is a chemical moiety having a free valence on a lead atom. Exemplary “lead groups” include, but are not limited to, —PbH₃, —PbH₂R, —PbHR₂, —PbR₃ and —Pb(OR)₃. In one aspect, each R can be independently a hydrocarbyl group; e.g. each R can be independently alkyl, cycloalkyl, aryl, aralkyl, substituted alkyl, substituted cycloalkyl, substituted aryl, or substituted aralkyl. In a “lead group” having more than one free valency, the other free valencies can be on any atom(s) in the group in accord with the rules of chemical structure and bonding, including atoms other than lead, for example, carbon.

A “boron group,” also called a “boron-bonded group,” is a generalized chemical moiety having at least one free valence on a boron atom. A “boronyl group” is a chemical moiety having at least one free valence on a boron atom. Exemplary “boronyl groups” include, but are not limited to, —BH₂, —BHR, —BR₂, —BR(OR), —B(OR)₂, and the like. In one aspect, each R can be independently a hydrocarbyl group; e.g. each R can be independently alkyl, cycloalkyl, aryl, aralkyl, substituted alkyl, substituted cycloalkyl, substituted aryl, or substituted aralkyl. In a “boron group” having more than one free valency, the other free valencies can be on any atom(s) in the group in accord with the rules of chemical structure and bonding, including atoms other than boron, for example, carbon.

An “aluminum group,” also called an “aluminum-bonded group,” is a generalized chemical moiety having at least one free valence on an aluminum atom. An “aluminyl group” is a chemical moiety having at least one free valence on an aluminum atom. Exemplary “aluminyl groups” include, but are not limited to, —AlH₂, —AlHR, —AlR₂, —AlR(OR), —Al(OR)₂, and the like. In one aspect, each R can be independently a hydrocarbyl group; e.g. each R can be independently alkyl, cycloalkyl, aryl, aralkyl, substituted alkyl, substituted cycloalkyl, substituted aryl, or substituted aralkyl. In an “aluminium group” having more than one free valency, the other free valencies can be on any atom(s) in the group in accord with the rules of chemical structure and bonding, including atoms other than aluminum, for example, carbon.

For each of the specific groups in which the free valence is situated on a heteroatom (non-carbon atom), such as the “oxygen group,” “sulfur group,” “nitrogen group,” “phosphorus group,” “arsenic group,” “silicon group,” “germanium group,” “tin group,” “lead group,” “boron group,” “aluminum group,” and the like, such groups can include a general “R” moiety. In each instance, R can be independently a organyl group; alternatively, a hydrocarbyl group; alternatively, an alkyl group; alternatively, an aliphatic group; alternatively, a cycloalkyl group; alternatively, an alkenyl group; alternatively, an alkynyl group; alternatively, an aromatic group; alternatively, an aryl group; alternatively, a heterocyclyl group; alternatively, a cycloheteryl group; alternatively, a heteroaryl group; alternatively, an arylheteryl group; alternatively, an organoheteryl group; alternatively, an aralkyl group; alternatively, a heteroaralkyl group; or alternatively, a halide.

An “organoaluminum compound,” is used to describe any compound that contains an aluminum-carbon bond. Thus, organoaluminum compounds include, but are not limited to, hydrocarbyl aluminum compounds such as trihydrocarbyl-, dihydrocarbyl-, or monohydrocarbylaluminum compounds; hydrocarbylaluminum halide compounds; hydrocarbylalumoxane compounds; and aluminate compounds which contain an aluminum-organyl bond such as tetrakis(p-tolyl)aluminate salts.

A “solid super acid” or “SSA” is synonymous with a solid oxide chemically-treated with an electron withdrawing anion, or a “chemically treated solid oxide” (CTSO). An SSA is a solid activator that derives from a solid oxide chemically-treated with an electron withdrawing anion as provided herein.

The term “substantially optically pure” is used to indicate a mixture of enantiomers having an enantiomeric excess of greater than or equal to 99.5%.

Terms that refer to the “substantial absence” of a component is intended to reflect a commercially-available sample of the recited components without the intentional addition of the specified component that is substantially absent. By way of example, the oligomerization reaction typically is carried out in an inert atmosphere that is “substantially free” of oxygen and/or water, meaning that engineering or laboratory methods to carry out reactions in which oxygen and/or water are excluded, such as drying solvents and using a dry, inert atmosphere such as dry nitrogen, or dry argon, are typically employed in the oligomerization reactor. For example, an inert atmosphere that is “substantially free” of oxygen and/or water can be interpreted to mean having less the 1,000, 750, 500, 250, 100, 75, 50, 25, 10, or 5 ppm oxygen and/or water. Reactions carried out in the substantial absence of aluminoxanes and/or organoborates are carried out without the addition of these components or the equivalent thereof, such as would be present if trialkyl aluminum compounds were intentionally contacted with water. For example, substantial absence of aluminoxanes and/or organoborates can be interpreted to mean having less than 5, 2.5, 1, 0.5, 0.1 weight percent aluminoxanes and/or organoborates.

The term “precontacted” is used herein to describe a first mixture of catalyst components that are contacted for a first period of time prior to the first mixture being used to form a “postcontacted” or second mixture of catalyst components that are contacted for a second period of time. For example, a precontacted mixture can describe a mixture of metallocene compound, olefin monomer, and organoaluminum compound, before this mixture is contacted with the chemically treated solid oxide and optionally additional organoaluminum compound. Thus, “precontacted” describes components that are used to contact each other, but prior to contacting with additional components in the second, postcontacted mixture. Accordingly, this disclosure can occasionally distinguish between a component used to prepare the precontacted mixture and that component after the mixture has been prepared. For example, according to this description, it is possible for the precontacted organoaluminum compound, once it is contacted with the metallocene and the olefin monomer, to have reacted to form at least one different chemical compound, formulation, or structure from the distinct organoaluminum compound used to prepare the precontacted mixture. In this case, the precontacted organoaluminum compound or component is described as comprising an organoaluminum compound that was used to prepare the precontacted mixture.

Similarly, the term “postcontacted” is used herein to describe a second mixture of catalyst components that are contacted for a second period of time, and one constituent of which is the “precontacted” or first mixture of catalyst components that were contacted for a first period of time. For example, a postcontacted mixture can describe a mixture of first metallocene compound, first metallocene compound, olefin monomer, organoaluminum compound, and chemically treated solid oxide, formed from contacting the precontacted mixture of a portion of these components with any additional components added to make up the postcontacted mixture. In this example, the additional component added to make up the postcontacted mixture is the chemically treated solid oxide, and optionally can include an organoaluminum compound the same or different from the organoaluminum compound used to prepare the precontacted mixture, as described herein. Accordingly, this disclosure can also occasionally distinguish between a component used to prepare the postcontacted mixture and that component after the mixture has been prepared.

The term “metallocene” as used herein is an organometallic coordination compound between a metal compound and at least one pi-bonded η^{x≧5} ligand; eg. η^{x≧5}-hydrocarbyl, η^{x≧5}-arene, η^{x≧5}-heteroarene, η^{x≧5}-heterocyclic, η^{x≧5}-organyl, or η^{x≧5}-organoheteryl group or moiety that is aromatic (for example, η⁵-cycloalkadienyl-type) or conjugated with (4n+2) pi-electrons, where n is an integer, usually either 1 or 2 (for example, η⁵-alkadienyl-type). In this aspect, the IUPAC definition (IUPAC Compendium of Chemical Terminology, 2^{nd} Edition (1997)) of a “metallocene” is much more limiting than the definition of a “metallocene” used herein; therefore the IUPAC definition for “metallocene” is not used herein. In this disclosure, such ligands can be referred to as Group I ligands, and compounds that contain at least one such ligand are referred to as metallocenes. For example, a metallocene can contain at least one pi-bonded η^{x≧5} ligand; e.g. η⁵-cycloalkadienyl-type or η⁵-alkadienyl-type ligand, for example, η⁵-cyclopentadienyl, η⁵-indenyl, η⁵-fluorenyl, η⁵-alkadienyl-, η⁶-boratabenzene-ligand, and the like. Thus, a metallocene is indicated as containing an η^{x≧5} moiety according to the usual if η^{x} (eta-x) nomenclature, in which x is an integer corresponding to the number of atoms which are coordinated to the transition metal or are expected to be coordinated to the transition metal, for example, according to the 18-electron rule.

The term “linking group” is used to describe the entire chemical moiety that connects two groups (for example, a Group I ligand with another ligand in the molecule, either another Group I ligand or a Group II ligand). The “linking group” includes a “bridge” having “bridging atom(s).” The bridge comprises the smallest number of contiguous atoms (bridging atoms) required to traverse the connection between the linked ligands (e.g. the Group I ligand and the other ligand it is connected to). Generally, the linking group and the bridge can comprise any atom; for example, the bridge can comprise C, Si, Ge, Sn, or any combination thereof. The linking group can be saturated, or the linking group can be unsaturated. By way of example, in the metallocene illustrated here, the “linking group” is the entire hydrocarbylene group C(CH₃)CH₂CH₂CH═CH₂, whereas the “bridge” or the “bridging atom” is a single carbon atom. Thus, the so-called “constrained-geometry” metallocene catalysts are encompassed within the metallocenes of the catalyst composition of this disclosure.

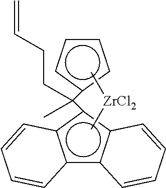

In some instances, reference can be made to “cyclic groups.” Unless otherwise specified, “cyclic groups” include aromatic and aliphatic groups having a ring structure, including homocyclic and heterocyclic groups.

### Olefin Wax Oligomer Composition

Generally, the olefin wax oligomer composition encompassed by the current disclosure minimally comprises olefin wax oligomers. That is to say compounds containing at least two olefin wax monomer units. However, since it is difficult to completely remove the olefin wax monomers from the olefin wax oligomers, the olefin wax oligomer compositions comprise, or consist essentially of, olefin wax monomers and olefin wax oligomers. Additionally, the properties of the olefin wax oligomer compositions will be the properties of the entire composition (olefin wax monomers plus olefin wax oligomers). In particular instances, the olefin wax oligomer may contain residual amounts (less than 1 weight percent) of catalyst system residues and/or deactivated catalyst system residues. Generally, these residues do not significantly impact the properties of the olefin wax oligomer composition.

Features which may be utilized to described the olefin wax oligomer compositions include the weight percent of olefin wax oligomers present in the composition, the weight percent of olefin wax monomers present in the composition, the 25° C. needle penetration of the olefin, the drop melt point of the composition, the 100° C. viscosity of the composition, the Mₙ as measured by GPC of the composition, the M_{w} as measured by GPC of the composition, the polydispersity index as measured by GPC of the composition, the olefin wax oligomer having the maximum peak height as measured by GPC, the olefin wax oligomer having the greatest peak area as measured by GPC, and/or the particular olefin wax utilized to produce the composition, among other features. These features are independently described herein and may be utilized in any combination to describe the olefin wax oligomer composition.

In an aspect, the olefin wax oligomer composition comprises greater than 40 percent olefin wax oligomers; alternatively, has greater than 50 weight percent olefin wax oligomers; alternatively, greater than 55 weight percent olefin wax oligomers; alternatively, greater than 60 weight percent olefin wax oligomers; alternatively, greater than 65 weight percent olefin wax oligomers; alternatively, greater than 70 weight percent olefin wax oligomers; alternatively, greater than 75 weight percent olefin wax oligomers; alternatively, greater than 80 weight percent olefin wax oligomers; alternatively, greater than 85 weight percent olefin wax oligomers; alternatively, greater than 90 weight percent olefin wax oligomers. In an embodiment, the olefin wax oligomer composition has from 40 to 95 weight percent olefin wax oligomers; alternatively, from 50 to 95 weight percent olefin wax oligomers; alternatively, 55 to 95 weight percent olefin wax oligomers; or alternatively, from 60 to 95 weight percent olefin wax oligomers alternatively, 65 to 95 weight percent olefin wax oligomers; or alternatively, from 70 to 95 weight percent olefin wax oligomers.

In an aspect, the olefin wax oligomer composition comprises the olefin wax oligomer composition has less than 50 weight percent olefin wax monomer; alternatively, less than 45 weight percent olefin wax monomer; alternatively, less than 40 weight percent olefin wax monomer; alternatively, less than 35 weight percent olefin wax monomer; alternatively, less than 30 weight percent olefin wax monomer; alternatively, less than 25 weight percent olefin wax monomer; alternatively, less than 20 weight percent olefin wax monomer; alternatively, less than 15 weight percent olefin wax monomer; or alternatively, less than 10 weight percent olefin wax monomer. In some embodiments, the olefin wax oligomer composition has from 5 to 60 weight percent olefin wax monomer; alternatively, from 5 to 50 weight percent olefin wax monomer; alternatively, from 5 to 45 weight percent olefin wax monomer; alternatively, from 5 to 40 weight percent olefin wax monomer; alternatively, from 5 to 35 weight percent olefin wax monomer; alternatively, from 5 to 30 weight percent olefin wax monomer.

The olefin wax oligomer content and olefin wax monomer content may be determined by GPC. Alternatively the olefin oligomer content and olefin wax monomer content may be determined by comparing the olefin wax monomer response of equal weight concentration solutions of the olefin wax and the olefin wax oligomer composition analyzed by gas chromatography.

In an aspect, the olefin wax oligomer composition has a needle penetration at least 5 percent lower than the needle penetration of the olefin wax monomer; alternatively, at least 10 percent lower than the needle penetration of the olefin wax monomer; alternatively, at least 15 percent lower than the needle penetration of the olefin wax monomer; alternatively, at least 20 percent lower than the needle penetration of the olefin wax monomer; alternatively, at least 25 percent lower than the needle penetration of the olefin wax monomer; alternatively, at least 30 percent lower than the needle penetration of the olefin wax monomer; alternatively, at least 35 percent lower than the needle penetration of the olefin wax monomer; alternatively, at least 40 percent lower than the needle penetration of the olefin wax monomer; alternatively, at least 45 percent lower than the needle penetration of the olefin wax monomer; alternatively, at least 50 percent lower than the needle penetration of the olefin wax monomer; alternatively, at least 55 percent lower than the needle penetration of the olefin wax monomer; alternatively, at least 60 percent lower than the needle penetration of the olefin wax monomer; alternatively, at least 65 percent lower than the needle penetration of the olefin wax monomer; alternatively, at least 70 percent lower than the needle penetration of the olefin wax monomer; alternatively, at least 75 percent lower than the needle penetration of the olefin wax monomer; alternatively, at least 80 percent lower than the needle penetration of the olefin wax monomer. Needle penetrations are measured at 25° C. (77° C.) according the procedure provided by ASTM D1321 and reported in units of dmm (decimillimeters).

In an aspect, the olefin wax oligomer composition has a drop melt point, in ° C., at least 5 percent higher than the olefin wax monomer; alternatively, at least 10 percent higher than the olefin wax monomer; alternatively, at least 15 percent higher than the olefin wax monomer; alternatively, at least 20 percent higher than the olefin wax monomer; alternatively, at least 25 percent higher than the olefin wax monomer; alternatively, at least 30 percent higher than the olefin wax monomer; alternatively, at least 35 percent higher than the olefin wax monomer; alternatively, at least 40 percent higher than the olefin wax monomer; alternatively, at least 45 percent higher than the olefin wax monomer; alternatively, at least 55 percent higher than the olefin wax monomer; or alternatively, at least 60 percent higher than the olefin wax monomer. Drop melt points are measured according the procedure provided by ASTM D127 and are reported in ° C.

In an aspect, the olefin wax oligomer composition has 100° C. kinematic viscosity at least 20 percent higher than the olefin wax monomer; alternatively, at least 40 percent higher than the olefin wax monomer; alternatively, at least 60 percent higher than the olefin wax monomer; alternatively, at least 80 percent higher than the olefin wax monomer; alternatively, at least 100 percent higher than the olefin wax monomer; alternatively, at least 120 percent higher than the olefin wax monomer; alternatively, at least 140 percent higher than the olefin wax monomer; alternatively, at least 160 percent higher than the olefin wax monomer; alternatively, at least 180 percent higher than the olefin wax monomer; or alternatively, at least 200 percent higher than the olefin wax monomer. The 100° C. kinematic viscosities are measured according the procedure provided by ASTM D445 and are reported in cSt.

In an aspect, the olefin wax oligomer composition has an Mₙ as measured by GPC greater than 1,000 g/mole; alternatively, greater than 1,250 g/mole; alternatively, greater than 1,500 g/mole; alternatively, greater than 1,750 g/mole; alternatively, greater than 2,000 g/mole; alternatively, greater than 2,250 g/mole; alternatively, greater than 2,500 g/mole; or alternatively, greater than 2,750 g/mole. In an embodiment, the olefin wax oligomer composition has an Mₙ as measured by GPC ranging from 1,000 g/mole to 50,000 g/mole; alternatively, ranging from 1,250 g/mole to 45,000 g/mole; alternatively, ranging from 1,500 g/mole to 40,000 g/mole; alternatively, ranging from 1,750 g/mole to 30,000 g/mole; alternatively, ranging from 2,000 g/mole to 20,000 g/mole; alternatively, ranging from 2,250 g/mole to 9,000 g/mole; alternatively, ranging from 2,500 g/mole to 15,000 g/mole; alternatively, ranging from 2,750 g/mole to 10,000 g/mole.

In an aspect, the olefin wax oligomer composition has an M_{w} greater than as measured by GPC greater than 4,000 g/mole; alternatively, greater than 6,000 g/mole; alternatively, greater than 7,000 g/mole; alternatively, greater than 8,000 g/mole; alternatively, greater than 9,000 g/mole; or alternatively, greater than 10,000 g/mole. In an aspect, the olefin wax oligomer composition has an M_{w} greater than as measured by GPC ranging from 2,000 g/mole to 500,000 g/mole; alternatively, ranging from 4,000 g/mole to 250,000 g/mole; alternatively, ranging from 6,000 g/mole to 150,000 g/mole; alternatively, ranging from 7,000 g/mole to 125,000 g/mole; alternatively, ranging from 8,000 g/mole to 100,000 g/mole; or alternatively, ranging from 9,000 g/mole to 75,000 g/mole; alternatively ranging from 10,000 g/mole to 50,000 g/mole.

In an aspect, the olefin wax oligomer composition has an polydispersity index as measured by GPC greater than 2; alternatively, greater than 2.5; alternatively, greater than 3; alternatively, greater than 3.5; alternatively, greater than 4; alternatively, greater than 5; alternatively, greater than 6; alternatively, greater than 7; or alternatively, greater than 8. In an embodiment, olefin wax oligomer composition has an polydispersity index as measured by GPC ranging from 2 to 16; alternatively, ranging from 2.5 to 15.5; alternatively, ranging from 3 to 15; alternatively, ranging from 3.5 to 14.5; alternatively, ranging from 4 to 14; alternatively, ranging from 5 to 13.5; alternatively, ranging from 6 to 13; alternatively, ranging from 7 to 12.5; or alternatively, ranging from 8 to 12.

In an aspect, the olefin wax oligomer having the greatest maximum peak height as measured by GPC has a molecular weight greater than 2,000 g/mole; alternatively, greater than 4,000 g/mole; alternatively, greater than 6,000 g/mole; alternatively, greater than 7,000 g/mole; alternatively, greater than 8,000 g/mole; or alternatively, greater than 9,000 g/mole. In an embodiment, the wax oligomer having the maximum peak height as measured by GPC has a molecular weight ranging from 2,000 g/mole to 100,000 g/mole; alternatively, ranging from 4,000 g/mole to 80,000 g/mole; alternatively, ranging from 6,000 g/mole to 70,000 g/mole; alternatively, ranging from 7,000 g/mole to 60,000 g/mole; alternatively, ranging from 8,000 g/mole to 50,000 g/mole; or alternatively, ranging from 9,000 g/mole to 40,000 g/mole. In an aspect, the olefin wax oligomer having the maximum peak area count as measured by GPC has a molecular weight greater than 2,000 g/mole; alternatively, greater than 4,000 g/mole; alternatively, greater than 6,000 g/mole; alternatively, greater than 7,000 g/mole; alternatively, greater than 8,000 g/mole; or alternatively, greater than 9,000 g/mole. In an embodiment, the wax oligomer having the maximum peak area count as measured by GPC has a molecular weight ranging from 2,000 g/mole to 100,000 g/mole; alternatively, ranging from 4,000 g/mole to 80,000 g/mole; alternatively, ranging from 6,000 g/mole to 70,000 g/mole; alternatively, ranging from 7,000 g/mole to 60,000 g/mole; alternatively, ranging from 8,000 g/mole to 50,000 g/mole; or alternatively, ranging from 9,000 g/mole to 40,000 g/mole.

According to a further aspect, the oil content of the olefin wax oligomer or olefin wax oligomer composition, as determined by methyl ethyl ketone (MEK) extraction, can be less than the oil content of the olefin wax. In some embodiments, for example, the oil content of the olefin wax oligomer composition is 80, 70, 60, 50, 40, 30, 25, 20, 15, 10, 5 wt. % of the olefin wax.

The olefin waxes, sometimes referred to as olefin wax monomer, which may be utilized to produce the olefin wax oligomer compositions are described herein and may be utilized, without limitation, to further described a olefin wax oligomer composition encompassed by this disclosure. In an exemplary, but non-limiting, embodiment, the olefin wax may be an alpha olefin wax; or alternatively, a normal alpha olefin wax. In one exemplary, but non-limiting, embodiment, the olefin wax is selected from the group consisting of an olefin wax having 70 wt % olefins having from 20 to 24 carbon atoms, an olefin wax having 60 wt % olefins having from 24 to 28 carbon atoms, an olefin wax having 70 wt % olefins having from 26 to 28 carbon atoms, and an olefin wax having 70 wt % olefins having greater than 30 carbon atoms. In another exemplary, but non-limiting embodiment, the olefin wax may be an olefin wax having 70 wt % olefins having from 20 to 24 carbon atoms; alternatively, an olefin wax having 60 wt % olefins having from 24 to 28 carbon atoms; alternatively, an olefin wax having 70 wt % olefins having from 26 to 28 carbon atoms; or alternatively, an olefin wax having 70 wt % olefins having greater than 30 carbon atoms. In a further exemplary, but non-limiting, embodiment, the olefin wax is selected from the group consisting of an olefin wax having 70 wt % olefins having from 20 to 24 carbon atoms and greater than 70 mole % alpha olefin, an olefin wax having 60 wt % olefins having from 24 to 28 carbon atoms and greater than 45 mole % alpha olefin, an olefin wax having 70 wt % olefins having from 26 to 28 carbon atoms and greater than 75 mole % alpha olefin, and an olefin wax having 70 wt % olefins having greater than 30 carbon atoms and greater than 45 mole % alpha olefin. In yet another exemplary, but non-limiting, embodiment, the olefin wax may be an olefin wax having 70 wt % olefins having from 20 to 24 carbon atoms and greater than 70 mole % alpha olefin; alternatively, an olefin wax having 60 wt % olefins having from 24 to 28 carbon atoms and greater than 45 mole % alpha olefin; alternatively, an olefin wax having 70 wt % olefins having from 26 to 28 carbon atoms and greater than 75 mole % alpha olefin; or alternatively, an olefin wax having 70 wt % olefins having greater than 30 carbon atoms and greater than 45 mole % alpha olefin. Other olefin waxes and olefin wax features are disclosed herein and may be utilized, without limitation, to describe the olefin wax.

### Olefin Wax

The terms olefin wax and olefin wax monomer may be used interchangeably to describe the olefin wax utilized in the method described herein and utilized to produce the olefin wax oligomer compositions described herein. Generally, but not-limiting, the term olefin wax is utilized to describe the olefinic material subjected to the methods described herein and olefin wax monomer refers to the unreacted components of the olefin wax found in the olefin wax oligomer compositions. With respect to using the word “monomer” in conjunction with an olefin wax, reference to a “monomer” encompasses the group of molecules in the olefin wax, and not to a single, specific molecule (e.g. a olefin wax having a specific carbon number). It is noted that the oligomerization catalyst systems described herein may have different oligomerization reactivities to different olefin wax isomers present in the olefin wax. Consequently, the olefin wax monomer present in the olefin wax composition can have a different olefin isomer distribution than found in the olefin wax from which the olefin wax composition was formed.

Generally, an olefin wax has at least 20 carbon atoms and at least one carbon-carbon double bond. In an embodiment, the olefin wax may be an alpha olefin wax. Generally, an alpha olefin is an olefin having a carbon-carbon double bond at the terminal position. In some embodiments the olefin wax may comprise internal olefins. In other embodiments, the olefin wax may comprise linear internal olefins. In yet other embodiments the olefin wax may be a normal alpha olefin wax. Additional criteria which may be independently applied, either singly or in any combination, to the olefin wax include the olefin wax composition's average olefin molecular weight, olefin wax composition carbon number composition, alpha olefin content, internal olefin content, linear internal olefin content, vinylidene olefin content, needle penetration, drop melt point, and viscosity, among others, are discussed below. The olefin wax may also contain paraffin wax. While the paraffin wax will not oligomerize under the method described herein, the paraffin wax is considered part of the olefin wax and will be present as unreacted material in the produced olefin wax oligomer composition.

In an embodiment, the olefin wax comprises greater than 30 mole % olefins having at least 20 carbon atoms. In some embodiments, the olefin wax comprises greater than 45 mole % olefins having at least 20 carbon atoms. In other embodiments, the olefin wax comprises greater than 60 mole % olefins having at least 20 carbon atoms. In a further embodiment, the olefin wax comprises greater than 75 mole % olefins having at least 20 carbon atoms. In yet a further embodiment, the olefin wax comprises greater than 90 mole % olefins having at least 20 carbon atoms. In still a further embodiment, the olefin wax comprises greater than 95 mole % olefins having at least 20 carbon atoms. In yet another embodiment, the olefin wax consists essentially of olefins having at least 20 carbon atoms. In an embodiment the olefins making up the olefin wax may be a hydrocarbon olefin.

The olefin wax's mole % olefin compositions are not limited to olefin waxes comprising olefins having at least 20 carbon atoms. The olefin mole % values may also be applied to any other olefin wax embodiments having any olefin carbon number, any carbon number range, and/or any average olefin molecular weight range described herein.

In an embodiment, the components of the olefin wax may include a paraffin wax in addition to the olefin wax. In some embodiments, the olefin wax may contain a paraffin wax having greater than 20 carbon atoms. In other embodiments, the olefin wax contains less than 65 mole % paraffins having greater than 20 carbon atoms; alternatively, less than 50 mole % paraffins having greater than 20 carbon atoms; alternatively, less than 35 mole % paraffins having greater than 20 carbon atoms; alternatively, less than 20 mole % paraffins having greater than 20 carbon atoms; alternatively, less than 8 mole % paraffins having greater than 20 carbon atoms; or alternatively, less than 5 mole % paraffins having greater than 20 carbon atoms.

The olefin wax's mole % paraffin contents are not limited to olefin waxes comprising olefins having at least 20 carbon atoms. The paraffin mole % values may also be applied to any other olefin wax embodiments having any olefin carbon number, any carbon number range, and/or any average olefin molecular weight range described herein.

In an embodiment, the olefin wax comprises alpha olefins. In one embodiment, the olefin wax comprises greater than 30 mole % alpha olefins having at least 20 carbon atoms. In some embodiments, the olefin wax comprises greater than 45 mole % alpha olefins having at least 20 carbon atoms. In other embodiments, the olefin wax comprises greater than 60 mole % alpha olefins having at least 20 carbon atoms. In a further embodiment, the olefin wax comprises greater than 75 mole % alpha olefins having at least 20 carbon atoms. In another embodiment, the olefin wax comprises greater than 90 mole % alpha olefins having at least 20 carbon atoms. In yet another embodiment, the olefin wax comprises greater than 95 mole % alpha olefins having at least 20 carbon atoms. In an embodiment, the alpha olefins which make the olefin wax may be a hydrocarbon alpha olefin. In another embodiment, the alpha olefins which make the olefin wax may be a normal alpha olefin.

The olefin wax mole % alpha olefins compositions are not limited to olefins having at least 20 carbon atoms. The alpha olefin mole % values may also be applied to any other olefin wax embodiments having any olefin carbon number, any carbon number range, and/or any average olefin molecular weight range described herein.

The olefin waxes comprising olefins and/or alpha olefins with carbon number distributions, alpha olefin contents, molecular weight distributions, and needle penetration values as described herein.

In one embodiment, the olefin wax comprises greater than 70 wt % olefins having from 20 to 24 carbon atoms. In a further embodiment, the olefin wax comprises greater than 80 wt % olefins having from 20 to 24 carbon atoms. In still a further embodiment, the olefin wax comprises greater than 85 wt % percent olefins having from 20 to 24 carbon atoms. In yet a further embodiment, the olefin wax comprises greater than 90 wt % olefins having from 20 to 24 carbon atoms. In still a further embodiment, the olefin wax comprises greater than 95 wt % olefins having from 20 to 24 carbon atoms.

In one embodiment, the olefin wax comprises greater than 50 wt % olefins having from 24 to 28 carbon atoms. In a further embodiment, the olefin wax comprises greater than 60 wt % olefins having from 24 to 28 carbon atoms. In a further embodiment, the olefin wax comprises greater than 70 wt % olefins having from 24 to 28 carbon atoms. In yet a further embodiment, the olefin wax comprises greater than 80 wt % olefins having from 24 to 28 carbon atoms. In still a further embodiment, the olefin wax comprises greater than 90 wt % olefins having from 24 to 28 carbon atoms.

In one embodiment, the olefin wax comprises greater than 50 wt % olefins having from 26 to 28 carbon atoms. In a further embodiment, the olefin wax comprises greater than 60 wt % olefins having from 26 to 28 carbon atoms. In a further embodiment, the olefin wax comprises greater than 70 wt % olefins having from 26 to 28 carbon atoms. In yet a further embodiment, the olefin wax comprises greater than 80 wt % olefins having from 26 to 28 carbon atoms. In still a further embodiment, the olefin wax comprises greater than 90 wt % olefins having from 26 to 28 carbon atoms.

In one embodiment, the olefin wax comprises greater than 70 wt % olefins having at least 30 carbon atoms. In a further embodiment, the olefin wax comprises greater than 80 wt % olefins having at least 30 carbon atoms. In still a further embodiment, the olefin wax comprises greater than 85 wt % percent olefins having from at least 30 carbon atoms. In yet a further embodiment, the olefin wax comprises greater than 90 wt % olefins having at least 30 carbon atoms. In still a further embodiment, the olefin wax comprises greater than 95 wt % olefins having at least 30 carbon atoms.

The olefin wax may alternatively be described as an olefin wax having a particular average molecular weight of the olefin components. In an embodiment, the olefin wax has an average olefin molecular weight greater than 260 grams/mole. In some embodiments, the olefin wax has an average olefin molecular weight greater than 330 grams/mole. In other embodiments, the olefin wax has an average olefin molecular weight greater than 400 grams/mole. In another embodiment, the olefin wax has an average olefin molecular weight between 260 grams/mole and 340 grams/mole; alternatively, between 280 grams/mole and 320 grams/mole; alternatively, between 290 grams/mole and 310 grams/mole. In a further embodiment, the olefin wax has an average olefin molecular weight between 330 grams/mole and 420 grams/mole; alternatively, between 350 grams/mole and 400 grams/mole; alternatively, between 360 grams/mole and 390 grams/mole. In yet another embodiment, the olefin wax has an average olefin molecular weight between 440 grams/mole and 550 grams/mole; alternatively, between 460 grams/mole and 530 grams/mole; alternatively, between 480 grams/mole and 510 grams/mole.

Commercially available olefin waxes (e.g. normal alpha olefin waxes) commonly contain a number of alpha olefins having at least 20 carbon atoms as well as other compounds (smaller alpha olefins, smaller normal alpha olefins, internal olefins, vinylidene, or others). For example, Alpha Olefin C₂₀₋₂₄ (ALPHAPLUS® C20-24, also designated C_{20/24} or C₂₀₋₂₄, Chevron Phillips Chemical Company LP, The Woodlands, Tex.) comprises from about 35-55 wt % C₂₀ olefin, about 25-45 wt % C₂₂ olefin, about 10-26 wt % C₂₄ olefin, about 3 wt % olefins smaller than C₂₀, and about 2 wt % olefins larger than C₂₄. Alpha Olefin C₂₀₋₂₄ is an exemplary olefin wax within the definition “comprising an olefin having at least 20 carbon atoms” as used herein. The various aspects of this disclosure are not limited to this or any other particular commercially available olefin wax. Also, an olefin wax consisting essentially of an olefin having 20 carbon atoms (or another olefin having a particular number of carbon atoms greater than 20) can also be used according to the present disclosure.

In one embodiment, the olefin wax comprises an olefin having from 20 carbon atoms to 24 carbon atoms. In a further embodiment, the olefin wax comprises an olefin having greater than 20 carbon atoms. In another embodiment, the olefin wax comprises an olefin having from 26 carbon atoms to 28 carbon atoms. In yet another embodiment, the olefin wax comprises an olefin having from 26 to 28 carbon atoms. In still an additional embodiment, the olefin wax comprises an olefin having at least 30 carbon atoms.

Commercially available olefin waxes may further comprise vinylidene or internal olefins, up to as much as about 40-50 wt % of the wax. In one embodiment, and regardless of the number of carbons in the olefin, the olefin wax is a high alpha (HA) AO wax. By “HA wax” is meant a wax comprising (a) one or more alpha olefins and (b) less than about 20 wt % vinylidene or internal olefins.

Independently, commercially available olefin wax compositions may further comprise non-olefin hydrocarbons, such as paraffins (hydrocarbons wherein all bonds between carbon atoms are single bonds). Other components known in the art to acceptably be present in olefin waxes can be present as well. For example, some applicable olefin waxes may contain oxygenated components such as alcohols, aldehydes, and ketones, among others.

Known olefin waxes include olefin streams from ethylene oligomerization, cracked heavy waxes (e.g. Fischer-Tropsch waxes), and mixtures of paraffins and olefins, among others. Additionally, the olefin waxes may include Fischer-Tropsch waxes comprising a mixture of paraffin waxes and olefin waxes which meet the described features of the olefin waxes described herein. One source of commercially available Fischer-Tropsch waxes is Sasol, Johannesburg, South Africa.

In some embodiments, the olefin wax may be a commercially available normal alpha olefin wax. In other embodiments, the olefin wax consists essentially of a commercially available normal alpha olefin waxes. One source of commercially available alpha olefin waxes is Chevron Phillips Chemical Company LP, The Woodlands, Tex., and alpha olefin waxes are available under the tradename ALPHAPLUS® normal alpha olefin (NAO) waxes, which may also be referred to herein as “Alpha Olefin” with a general designation of the range of olefin size as the principal components. For example, ALPHAPLUS® C20-24 (also designated C_{20/24} or C₂₀₋₂₄) may be designated “Alpha Olefin C₂₀₋₂₄”, ALPHAPLUS® C24-28 (C_{24/28} or C₂₄₋₂₈) may be designated “Alpha Olefin C₂₄₋₂₈”, ALPHAPLUS® C26-28 (C26/28 or C26-28) may be designated “Alpha Olefin C₂₆₋₂₈”, the high alpha (HA) AO wax ALPHAPLUS® C30+HA (C_{30+HA}) may be designated “Alpha Olefin C_{30+HA}”, and ALPHAPLUS® C30+ (C₃₀₊) may be designated “Alpha Olefin C₃₀₊”, where the carbon number indicates the highest proportion of olefins in the product. In an embodiment, the olefin wax may consist essentially of Alpha Olefin C₂₀₋₂₄; alternatively, Alpha Olefin C₂₄₋₂₈; alternatively, Alpha Olefin C₂₆₋₂₈; alternatively, Alpha Olefin C₃₀₊; or alternatively, Alpha Olefin C_{30+HA} The following are published physical and chemical characteristics of the normal alpha olefin waxes Alpha Olefin C₂₀₋₂₄, Alpha Olefin C₂₄₋₂₈, Alpha Olefin C₂₆₋₂₈, Alpha Olefin C₃₀₊, and Alpha Olefin C_{30+HA}, which are provided for illustrative purposes as exemplary feedstock olefin waxes. The various aspects of this disclosure are not limited to these particular feedstock olefin waxes.

| | Typical Value (Typical Range) | | | | |
|---|---|---|---|---|---|
| Characteristic | C₂₀₋₂₄ | C₂₄₋₂₈ | C₂₆₋₂₈ | C₃₀₊ | C_{30+HA} |
| Drop melt point, ° F. | 96 | 143 | 125 | 162 | 159 |
| (ASTM D 127) | (ASTM D 87) | (140-158) | (122-130) | (154-174) | (150-164) |
| Oil content (MEK | | 3.7 | 3.8 | 1.50 | 1.5 |
| extraction), wt. % | | (3.0-5.1) | (3.2-5.3) | (1.0-2.0) | (1.2-3.0) |
| Needle Penetration | 150 | 59 | 48 | 13 | 15.5 |
| @ 77° F., dmm | | (48-70) | (40-60) | (11-17) | (12-18) |
| Needle Penetration | | | | 24 | 32 |
| @ 100° F., dmm | | | | (18-30) | (24-44) |
| Needle Penetration | | | | 34 | 40 |
| @ 110° F., dmm | | | | (25-50) | (30-56) |
| Flash Point | 362° F. | 425° F. | 417° F. | 485° F. | 432° F. |
| (ASTM D 93) | (183° C.) | (218° C.) | (214° C.) | (252° C.) | (222° C.) |
| Saybolt Color | 30 | 25 | 30 | 20+ | 20+ |
| Kinematic Viscosity | 2.0 | 3.5 | 3.4 | 6.5 | 6.4 |
| @ 100° C., cSt | (1.8-2.2) | (3.2-4.0) | (3.2-3.6) | (5.0-10.0) | (5.0-9.0) |
| % Alpha olefins | 86 | 54 | 79 | 62 | 76 |
| (¹H-NMR) | (83-92) | (40-60) | (70-82) | (50-65) | (70-81) |
| % Vinylidenes | 8 | 30 | 16 | 30 | 18 |
| (¹H-NMR) | (6-15) | (25-55) | (11-17) | (25-45) | (15-25) |
| % Internal olefins | 3 | 18 | 3 | 10 | 5.3 |
| (¹H-NMR) | (2-5) | (10-22) | (2-8) | (5-20) | (4-10) |
| Drop melt point, ° F. | 96 | 154 | 125 | 164 | 150 |
| (ASTM D 127) | | | | | |
| Oil content (MEK | | 4.60 | 5.00 | 1.50 | 1.5 |
| extraction), wt. % | | | | | |
| Needle Penetration | 150 | 59 | 48 | 13 | 15.5 |
| @ 77° F., dmm | | | | | |
| Needle Penetration | | | | 24 | 32 |
| @ 100° F., dmm | | | | | |
| Needle Penetration | | | | 34 | 40 |
| @ 110° F., dmm | | | | | |
| Flash Point | 362° F. | 425° F. | 417° F. | 485° F. | 432° F. |
| (ASTM D 93) | (183° C.) | (218° C) | (214° C.) | (252° C.) | (222° C.) |
| Saybolt Color | 30 | 25 | 30 | 20+ | 20+ |

### The Catalyst System and Components

This disclosure encompasses a catalyst system, a method of making the catalyst system, an oligomerization method using the catalyst system, and a method of producing an olefin wax oligomer and/or an olefin wax oligomer composition using the catalyst system. In one aspect the disclosed catalyst system generally comprises a metallocene component. According to another aspect, the catalyst system can comprise a metallocene and an activator component. The activator component itself can comprise one, two, three, or more activators. For example, the catalyst system can comprise at least one metallocene and at least one activator; alternatively, the catalyst system can comprise at least one metallocene, at least one first activator, and at least one second activator.

This disclosure also encompasses an oligomerization method comprising: a) contacting an olefin wax and a catalyst system comprising a metallocene, and b) forming an olefin wax oligomer and/or an olefin wax oligomer composition under oligomerization conditions. Alternatively, this disclosure encompasses an oligomerization method comprising: a) contacting an olefin wax and a catalyst system comprising a metallocene and an activator, and b) forming an olefin wax oligomer and/or an olefin wax oligomer composition under oligomerization conditions. For example, in other embodiments, the catalyst system can comprise a metallocene, a first activator, and a second activator. In other embodiments, or the catalyst system can be substantially devoid of an activator.

Generally, the olefin wax, the catalyst system, metallocene, activator (first, second, or other), the olefin wax oligomer and/or an olefin wax oligomer composition, the oligomerization conditions, and the like are independent elements of the oligomerization method and are independently described herein. The oligomerization method and any process which incorporates the oligomerization method can be described utilizing any combination of olefin wax described herein, catalyst system described herein, metallocene described herein, activator (first, second, or other) described herein, olefin wax oligomer and/or an olefin wax oligomer composition described herein, oligomerization conditions described herein, and the like.

When an activator is used in the catalyst system, the activator (first, second, or other) can comprise a solid oxide chemically-treated with an electron withdrawing anion; alternatively, the activator (first, second, or other) can comprise, consist essentially of, or consist of, an alumoxane. In some embodiments, the catalyst system can comprise, consist essentially of, or consist of, a metallocene, a first activator comprising a solid oxide chemically-treated with an electron withdrawing anion, and a second activator. In another embodiment, the catalyst system can comprise, consist essentially of, or consist of, a metallocene, a first activator comprising (consisting essentially of, or consisting of) an alumoxane. In other embodiments, the catalyst system can comprise consist essentially of, or consist of, a metallocene, a first activator an alumoxane, and a second activator.

In exemplary, but non-limiting, embodiments, the catalyst system can comprise consist essentially of, or consist of, a metallocene; alternatively, a metallocene and an aluminoxane; alternatively, a metallocene and a chemically-treated solid oxide; alternatively, a metallocene, an aluminoxane, and a chemically-treated solid oxide. In further exemplary, but non-limiting, embodiments, the catalyst system can comprise a metallocene, a chemically-treated solid oxide, and an organoaluminum compound; alternatively, a metallocene, a chemically-treated solid oxide, and an organoboron compound; alternatively, a metallocene, a chemically-treated solid oxide, and an organozinc compound; alternatively, a metallocene, a chemically-treated solid oxide, and an organomagnesium compound; alternatively, a metallocene, a chemically-treated solid oxide, and an organolithium compound; or alternatively, a metallocene, a chemically-treated solid oxide, and an ionizing ionic compound. In further exemplary, but non-limiting, embodiments, the catalyst system can comprise consist essentially of, or consist of, a metallocene and any combination of an aluminoxane, a chemically-treated solid oxide, an organoaluminum compound, an organoboron compound, an organozinc compound, an organomagnesium compound, an organolithium compound, and/or an ionizing ionic compound.

In further embodiments, exemplary activator(s) that can be used in conjunction with a metallocene include: 1) an aluminoxane; 2) a chemically-treated solid oxide; 3) a chemically-treated solid oxide in combination with any one or more organoaluminum compound, organoboron compound, organozinc compound, organomagnesium compound, organolithium compound, and/or ionizing ionic compound.

Any number of precontacting or postcontacting steps can be employed in which any selection of catalyst system components and/or the olefin wax monomer can be precontacted and/or postcontacted prior to the step of forming olefin wax oligomer product under oligomerization conditions. In any aspect or embodiment of the oligomerization method disclosed herein can utilize any combination of olefin wax monomer, metallocene, activator, solid oxide, or electron withdrawing anion, or any other activator or combination of activators which can be precontacted for any length of time prior to the step of contacting the olefin wax and the catalyst system. Each of the components that can be used in the catalyst system is described independently herein.

In an aspect and any embodiment described herein, the oligomerization method(s) described herein can be incorporated into a process of producing an olefin wax oligomer and/or an olefin wax oligomer composition. In an non-limiting embodiment, the process to produce an olefin wax oligomer and/or an olefin wax oligomer composition comprises: a) contacting an olefin wax and a catalyst system comprising a metallocene, and b) forming an olefin wax oligomer and/or olefin wax oligomer composition under oligomerization conditions.

According to a further exemplary, but non-limiting, embodiment, this disclosure further encompasses a method of producing an olefin wax oligomer and/or an olefin wax oligomer composition, a method of oligomerizing an olefin wax, and/or a method of producing any olefin wax oligomer and/or any olefin wax oligomer composition described herein. Generally, the methods comprise:

a) contacting an olefin wax and a catalyst system; and

b) oligomerizing the olefin wax under oligomerization conditions.

The olefin wax, the catalyst system, and the oligomerization conditions are independent elements of the method and their description found herein may be utilized in any combination to further describe the method encompassed by this disclosure.

These and other elements of the catalyst systems along with other features (e.g. ratio of catalyst system components) of the catalyst system encompassed by this disclosure are further described herein and may be utilized, without limitation, to further describe the catalyst system.

### The Metallocene Component

In one aspect, the present disclosure provides a catalyst system comprising a metallocene. In an embodiment, a combination of metallocenes can be employed in the catalysts system. When multiple metallocenes are utilized, the metallocene may be referred to herein as a first metallocene (or metallocene compound) and a second metallocene (or metallocene compound). In another aspect, two different metallocenes can be used simultaneously in an oligomerization process to produce the alpha olefin product.

Throughout this disclosure, metallocenes are described generally as comprising a Group I ligand, a Group II ligand, and a group 4, 5, or 6 metal; alternatively, a Group I ligand, a Group II ligand, and a group 4 metal; alternatively, a Group I ligand, a Group II ligand, and a group 5 metal; or alternatively, a Group I ligand, a Group II ligand, and a group 6 metal. In an aspect, the metal of the metallocene can be Ti, Zr, Hf, V, Nb, Ta, Cr, Mo, or W. In another aspect, the metal of the metallocene can be titanium, zirconium, hafnium, vanadium, niobium, tantalum, chromium, molybdenum, or tungsten; alternatively, titanium, zirconium, hafnium, or vanadium; alternatively, titanium, zirconium, or hafnium; alternatively, titanium; alternatively, zirconium; alternatively, hafnium; or alternatively, vanadium.

In an aspect, the Group I ligands of the metallocene are pi-bonded η^{x≧5} ligands. The pi-bonded η^{x≧5} ligands which can be utilized as a Group I ligand of the present disclosure include η⁵-cycloalkadienyl-type ligands, η⁵-cycloalkadienyl-type ligand analogs, and η⁵-alkadienyl-type ligands as utilized in “open metallocenes.” In an embodiment, a metallocene which can be utilized in any aspect or embodiment of the present disclosure contains at least one η⁵-cycloalkadienyl-type or η⁵-alkadienyl-type ligand. In some embodiments, the Group I ligand can be η⁵-cyclopentadienyl, η⁵-indenyl, η⁵-fluorenyl, η⁵-alkadienyl-, η⁶-boratabenzene-ligand, and their substituted analogs. Other aspects and embodiments of the Group I ligands are described herein and can be utilized without limitation to describe the metallocene with can be utilized in any aspect or embodiment disclosed herein. Regarding the bonding of the unsaturated ligand to the metal in a metallocene, such a ligand can be indicated as containing a ligand bound according to the usual if η^{x} (eta-x) nomenclature, in which x is an integer corresponding to the number of atoms which are coordinated to the transition metal or are expected to be coordinated to the transition metal, for example, according to the 18-electron rule. The Group I ligands can be substituted or unsubstituted.

According to a further aspect, the Group I ligands can comprise at least one heterocyclic ring that is fused to a η⁵-cycloalkadienyl-type or η⁵-alkadienyl-type ligand. In some embodiments, for example, the Group I ligand can be a η⁵-cyclopentadienyl ligand, a η⁵-indenyl ligand, or similar Group I ligands, including their substituted analogs, to which a heterocyclic moiety is fused. Examples of fused heterocyclic moieties include, but are not limited to, pyrrole, furan, thiophene, phosphole, imidazole, imidazoline, pyrazole, pyrazoline, oxazole, oxazoline, isoxazole, isoxazoline, thiazole, thiazoline, isothiozoline, and the like, including partially saturated analogs of these rings.

In an aspect, the Group II ligands of the metallocene are the ligands that are not η^{x≧5} bonded ligands and are prototypically sigma-bonded ligands and those pi-bonded ligands that are bound to the metal in an η^{x≧5} bonding mode. Therefore, the η^{x≧5}-bonded ligands encompass the typical sigma-bonded halide, sigma-bonded hydride, sigma-bonded hydrocarbyl ligands (e.g. alkyl and alkenyl ligands, among others), and η^{x≧5} “pi-bonded” ligands such as η²-alkene, η⁴-alkadienyl, and the like, which are bound to the metal in an η^{x≧5} bonding mode. Thus, the Group II ligand of the metallocenes of this disclosure include those sigma-bonded ligands and some pi-bonded ligands in the metallocene that are not the η⁵-cycloalkadienyl-type ligands and are not the other pi-bonded η^{x≧5} ligands typically associated with defining a metallocene compound. Examples and alternative embodiments of Group II ligands are provided herein.

In an aspect, the metallocene can comprise two Group I ligands. In this aspect, and in any embodiment, the metallocene can comprise two Group I ligands, wherein the two Group I ligands are connected by a linking group; or alternatively, wherein the two Group I ligands are separate (not connected or unlinked). Because a linking group is considered a substituent on a Group I ligand, a linked Group I ligand can be further substituted with other, non-linking substituents or can be unsubstituted with the exception of the linking group. Thus, the Group I ligands can be linked and further substituted, linked but not further substituted, not linked but substituted with non-linking ligands, or not linked and not further substituted; alternatively, the Group I ligands can be linked and further substituted; alternatively, the Group ligands can be linked but not further substituted; alternatively, the Group I ligands may not be linked but substituted with non-linking ligands; or alternatively, the Group I ligands may not be linked and not further substituted. Also in any embodiment, the metallocene can comprise a Group I ligand and at least one Group II ligand, where the Group I ligand and a Group II ligand are connected by a linking group; or alternatively, where the Group I ligand the Group II ligands are separate and not connected by a linking group.

In an aspect, and in any embodiment, the metallocene can have the formula X²¹X²²X²³X²⁴M¹. In this aspect, X²¹, X²², X²³, X²⁴, and M¹ are independently described herein and can be utilized in any combination to described the metallocene having the formula X²¹X²²X²³X²⁴M¹. In some embodiments, M¹ can be a group 4, 5, or 6 metal; alternatively, a group 4 metal; alternatively, a group 5 metal; or alternatively, a group 6 metal. In other embodiments, M¹ can be Ti, Zr, Hf, V, Nb, Ta, Cr, Mo, or W; alternatively, Ti, Zr, or Hf; alternatively, V, Nb, or Ta; alternatively, Cr, Mo, or W; alternatively, Ti, Zr, Hf, or V; alternatively, Ti, Zr, or Hf; alternatively, Ti; alternatively, Zr; alternatively, Hf; or alternatively, V. In an embodiment, X²¹ is a Group I ligand, X²² is a Group I ligand or a Group II ligand, and X³ and X⁴ independently are Group II ligands; alternatively, X²¹ and X²² independently are Group I ligands not connected by a linking group, and X²³ and X²⁴ independently are Group II ligands; alternatively, X²¹ and X²² independently are Group I ligands connected by a linking group, and X²³ and X²⁴ independently are Group II ligands; or alternatively, X²¹ is a Group I ligand and X²², X²³, and X²⁴ independently are substituted or an unsubstituted hydrocarbyl group having from 1 to 20 carbon atoms. In an embodiment, any substituent on X²¹, X²², X²³, and X²⁴ can be independently a halide, a C₁ to C₂₀ hydrocarboxide group, an C₁ to C₂₀ aliphatic group, a C₁ to C₂₀ heterocyclic group, a C₆ to C₂₀ aromatic group, a C₁ to C₂₀ heteroaromatic group, an amido group, an C₁ to C₂₀ N-hydrocarbylamido group, a C₁ to C₂₀ N,N-dihydrocarbylamido group, a C₁ to C₂₀ hydrocarbylthiolate group, or a C₃ to C₃₀ trihydrocarbylsiloxy group.

In a non-limiting embodiment, the metallocene can have the formula:

X²¹X²²X²³X²⁴M¹; wherein:
M¹ is selected from titanium, zirconium, hafnium, vanadium, niobium, tantalum, chromium, molybdenum, or tungsten;
X²¹ is a Group I ligand;
X²² is a Group I ligand or a Group II ligand; and
X²³ and X²⁴ are independently selected from a Group II ligand. In some embodiments X²¹ and
X²² are connected by a linking group. In other embodiments, X²¹ and X²² are not connected by a linking group.
   In some non-limiting embodiments, the metallocene can have the formula:

X²¹X²²X²³X²⁴M¹: wherein:
M¹ is selected independently from Ti, Zr, or Hf;
X²¹ and X²² are Group I ligands connected by a linking group; and
X²³ and X²⁴ are independently selected from a Group II ligand.
   In other non-limiting embodiments, the metallocene can have the formula:

X²¹X²²X²³X²⁴M¹: wherein:
M¹ is selected independently from Ti, Zr, or Hf;
X²¹ and X²² are Group I ligands not connected by a linking group; and
X²³ and X²⁴ are independently selected from a Group II ligand.
   In yet another non-limiting embodiment, the metallocene can have the formula:

X²⁵X²⁶X²⁷X²⁸M²; wherein
M² is Ti, Zr, Hf, or V;
X²⁵ is a Group I ligand;
X²⁶, X²⁷, and X²⁸ are selected independently from a substituted or an unsubstituted hydrocarbyl group having from 1 to 20 carbon atoms; and wherein
any substituent on X²⁵, X²⁶, X²⁷, and X²⁸ can be independently a halide, a C₁ to C₂₀ hydrocarboxide group, an C₁ to C₂₀ aliphatic group, a C₁ to C₂₀ heterocyclic group, a C₆ to C₂₀ aromatic group, a C₁ to C₂₀ heteroaromatic group, an amido group, an C₁ to C₂₀ N-hydrocarbylamido group, a C₁ to C₂₀ N,N-dihydrocarbylamido group, a C₁ to C₂₀ hydrocarbylthiolate group, and a C₃ to C₃₀ trihydrocarbylsiloxy group.

In one aspect, and in any embodiment, the metallocene can include a linking group that connects a Group I ligand with another ligand (either another Group I ligand or a Group II ligand) in the metallocene. The linking group includes a bridge, comprising the smallest number of contiguous atoms required to traverse the connection between the Group I ligand and the other ligand it is connected to. For example, the linking group can comprise from 1 to 3 contiguous bridging atoms; alternatively, 1 or 2 contiguous bridging atoms; alternatively, 1 bridging atom; alternatively, 2 contiguous bridging atoms; alternatively, 3 contiguous bridging atoms. In an embodiment, each contiguous bridging atom can be C, O, S, N, P, Si, Ga, Sn, or Pb; alternatively, C, Si, Ge, or Sn; alternatively; C or Si; alternatively, C; or alternatively, Si. The linking group can be saturated, or the linking group can be unsaturated; alternatively, linking group can be saturated; or alternatively, linking group can be unsaturated.

Linking groups include, but are not limited to, a C₁-C₂₀ hydrocarbyl group, a C₀-C₂₀ nitrogen-bonded group, a C₀-C₂₀ phosphorus-bonded group, a C₁-C₂₀ organyl group, a C₀-C₃₀ silicon-bonded group, a C₀-C₂₀ germanium-bonded group, a C₀-C₂₀ tin-bonded group, or a C₀-C₂₀ lead-bonded group; alternatively, a C₁-C₂₀ hydrocarbyl group, or a C₀-C₃₀ silicon-bonded group; alternatively, a C₁-C₂₀ hydrocarbyl group; alternatively, a C₀-C₂₀ nitrogen-bonded group; alternatively, a C₀-C₂₀ phosphorus-bonded group; alternatively, a C₁-C₂₀ organyl group; alternatively, a C₀-C₃₀ silicon-bonded group; alternatively, a C₀-C₂₀ germanium-bonded group; alternatively, a C₀-C₂₀ tin-bonded group; or alternatively, a C₀-C₂₀ lead-bonded group.

Linking groups in any aspect or embodiment comprising linking groups, include those moieties having the formula >CR¹R², >SiR³R⁴, or —CR⁵R⁶CR⁷R⁸—, where R¹, R², R³, R⁴, R⁵, R⁶, R⁷, and R⁸ are selected independently from a hydrogen, a halide, a C₁-C₂₀ hydrocarbyl group, a C₁-C₂₀ oxygen-bonded group, a C₁-C₂₀ sulfur-bonded group, a C₀-C₂₀ nitrogen-bonded group, a C₀-C₂₀ phosphorus-bonded group, a C₁-C₂₀ organyl group, a C₀ to C₂₀ arsenic-bonded group, a C₀-C₂₀ silicon-bonded group, a C₀-C₂₀ germanium-bonded group, or a C₀-C₂₀ tin-bonded group; a C₀ to C₂₀ lead-bonded group, a C₀ to C₂₀ boron-bonded group, or a C₀ to C₂₀ aluminum-bonded group. In this aspect and in any embodiment, R¹, R², R³, R⁴, R⁵, R⁶, R⁷, and R⁸ can be, independently, saturated or unsaturated; alternatively, saturated; or alternatively, unsaturated. In some embodiments comprising linking groups, the linking group can have the formula >CR¹R², >SiR³R⁴, or —CR⁵R⁶CR⁷R⁸—, in which R¹, R², R³, R⁴, R⁵, R⁶, R⁷, and R⁸ are selected independently from a hydrogen, a halide, a saturated or unsaturated C₁-C₂₀ aliphatic group, or a C₆-C₂₀ aromatic group; alternatively, a saturated C₁-C₂₀ aliphatic group; alternatively, R¹, R², R³, R⁴, R⁵, R⁶, R⁷, and R⁸ can be selected independently from a hydrogen, a halide, a C₁-C₂₀ alkyl group, a C₂-C₂₀ alkenyl group, a C₂-C₂₀ alkynyl group, a C₆-C₂₀ aryl group, or a C₆-C₂₀ aromatic group; alternatively, a hydrogen, a C₁-C₂₀ alkyl group, a C₂-C₂₀ alkenyl group, or a C₆-C₂₀ aryl group; or alternatively, R¹, R², R³, R⁴, R⁵, R⁶, R⁷, and R⁸ are selected independently from a hydrogen, or saturated or unsaturated C₁-C₂₀ hydrocarbyl group. Hydrocarbyl, aliphatic, alkyl, alkenyl, alkynyl, aryl, and aromatic groups are described herein and can be utilized to describe R¹, R², R³, R⁴, R⁵, R⁶, R⁷, and/or R⁸ which can be utilized in the liking groups.

In yet another aspect and in any embodiment, in each occurrence of the Group I ligand in a metallocene can be a substituted or an unsubstituted η⁵-cycloalkadienyl-ligand, a substituted or an unsubstituted η⁵-alkadienyl-ligand, or a substituted or an unsubstituted η⁶-boratabenzene-containing ligand; alternatively, a substituted or an unsubstituted cyclopentadienyl ligand, a substituted or an unsubstituted indenyl ligand, a substituted or an unsubstituted fluorenyl ligand, a substituted or an unsubstituted tetrahydroindenyl ligand, a substituted or an unsubstituted tetrahydrofluorenyl ligand, or a substituted or an unsubstituted octahydrofluorenyl ligand; or alternatively, a substituted or an unsubstituted cyclopentadienyl ligand, a substituted or an unsubstituted indenyl ligand, or a substituted or an unsubstituted fluorenyl ligand. Further, in any embodiment, in each occurrence of the Group I ligand in a metallocene a substituted or an unsubstituted cyclopentadienyl; alternatively, substituted or an unsubstituted indenyl; alternatively, substituted or an unsubstituted fluorenyl; alternatively, substituted or an unsubstituted tetrahydroindenyl; alternatively, substituted or an unsubstituted tetrahydrofluorenyl; or alternatively, a substituted or an unsubstituted octahydrofluorenyl. Alternatively, the metallocene can have two Group 1 ligands and in each occurrence of the Group I ligand, the Group I ligand can be independently two substituted or unsubstituted cyclopentadienyls, a substituted or an unsubstituted fluorenyl and a substituted or an unsubstituted cyclopentadienyl, a substituted or an unsubstituted fluorenyl and a substituted or an unsubstituted indenyl, two substituted or unsubstituted fluorenyls or two substituted or unsubstituted indenyls. Alternatively, in each occurrence of the Group I ligand, the Group I ligand can be selected independently from a substituted or an unsubstituted cyclopentadienyl, a substituted or an unsubstituted indenyl, or a substituted or an unsubstituted fluorenyl.

As disclosed herein, a linked Group I ligand can be further substituted with other, non-linking substituents or can be further unsubstituted. A non-linked Group I ligand can be substituted or can be unsubstituted. In this aspect, each non-linking substituent on a Group I ligand can be independently, but is not limited to, a halide, a C₁ to C₂₀ hydrocarbyl group, a C₁ to C₂₀ hydrocarboxy group, a C₃ to C₂₀ heterocyclic group, a C₆ to C₂₀ aromatic group, a C₃ to C₂₀ heteroaromatic group, a C₁ to C₂₀ hydrocarbylsilyl group, a C₂ to C₄₀ dihydrocarbylsilyl group, a C₃ to C₆₀ trihydrocarbylsilyl group, an aminyl group, a C₁ to C₂₀ N-hydrocarbyl aminyl group (sometimes referred to as a C₁ to C₂₀ N-hydrocarbylamido group), a C₂ to C₄₀ N,N-dihydrocarbyl aminyl group (sometimes referred to as a C₂ to C₄₀ N,N-dihydrocarbylamido group), a C₁ to C₂₀ hydrocarbylthiolate group, or a C₃ to C₆₀ trihydrocarbylsiloxy group; alternatively, a halide, a C₁ to C₂₀ hydrocarbyl group, or a C₁ to C₂₀ hydrocarboxy group; alternatively, a halide or a C₁ to C₂₀ hydrocarbyl group; alternatively, a halide or a C₁ to C₂₀ hydrocarboxy group; alternatively, a C₁ to C₂₀ hydrocarbyl group or a C₁ to C₂₀ hydrocarboxy group; alternatively, a halide; alternatively, a C₁ to C₂₀ hydrocarbyl group; or alternatively, a C₁ to C₂₀ hydrocarboxy group. In another aspect and any embodiment disclosed herein each non-linking substituent on a Group I ligand can be independently, but is not limited to, a halide, a C₁ to C₁₀ hydrocarbyl group, a C₁ to C₁₀ hydrocarboxy group, a C₃ to C₁₅ heterocyclic group, a C₆ to C₁₅ aromatic group, a C₃ to C₁₅ heteroaromatic group, a C₁ to C₁₀ hydrocarbylsilyl group, a C₂ to C₂₀ dihydrocarbylsilyl group, a C₃ to C₃₀ trihydrocarbylsilyl group, an aminyl group, a C₁ to C₁₀ N-hydrocarbyl aminyl group (sometimes referred to as a C₁ to C₁₀ N-hydrocarbylamido group), a C₂ to C₂₀ N,N-dihydrocarbyl aminyl group (sometimes referred to as a C₂ to C₂₀ N,N-dihydrocarbylamido group), a C₁ to C₁₀ hydrocarbylthiolate group, or a C₃ to C₃₀ trihydrocarbylsiloxy group; alternatively, a halide, a C₁ to C₁₀ hydrocarbyl group, or a C₁ to C₁₀ hydrocarboxy group; alternatively, a halide or a C₁ to C₁₀ hydrocarbyl group; alternatively, a halide or a C₁ to C₁₀ hydrocarboxy group; alternatively, a C₁ to C₁₀ hydrocarbyl group or a C₁ to C₁₀ hydrocarboxy group; alternatively, a halide; alternatively, a C₁ to C₁₀ hydrocarbyl group; or alternatively, a C₁ to C₁₀ hydrocarboxy group.

In yet another aspect and any embodiment disclosed herein, each non-linking substituent on a Group I ligand can be independently, but is not limited to, a halide, a C₁ to C₅ hydrocarbyl group, a C₁ to C₅ hydrocarboxy group, a C₃ to C₁₀ heterocyclic group, a C₆ to C₁₀ aromatic group, a C₃ to C₁₀ heteroaromatic group, a C₁ to C₅ hydrocarbylsilyl group, a C₂ to C₁₀ dihydrocarbylsilyl group, a C₃ to C₁₅ trihydrocarbylsilyl group, an aminyl group, a C₁ to C₅ N-hydrocarbyl aminyl group (sometimes referred to as a C₁ to C₅ N-hydrocarbylamido group), a C₂ to C₁₀ N,N-dihydrocarbyl aminyl group (sometimes referred to as a C₂ to C₁₀ N,N-dihydrocarbylamido group), a C₁ to C₅ hydrocarbylthiolate group, or a C₃ to C₁₅ trihydrocarbylsiloxy group; alternatively, a halide, a C₁ to C₅ hydrocarbyl group, or a C₁ to C₅ hydrocarboxy group; alternatively, a halide or a C₁ to C₅ hydrocarbyl group; alternatively, a halide or a C₁ to C₅ hydrocarboxy group; alternatively, a C₁ to C₅ hydrocarbyl group or a C₁ to C₅ hydrocarboxy group; alternatively, a halide; alternatively, a C₁ to C₅ hydrocarbyl group; or alternatively, a C₁ to C₅ hydrocarboxy group.

In an embodiment, each halide substituent which may be utilized as non-linking substituent on a Group I ligand or as a halide utilized in a linking group can be independently a fluoride, a chloride, a bromide, or an iodide. In an embodiment, each halide substituent which may be utilized as non-linking substituent on a Group I ligand or as a halide utilized in a linking group can be independently a fluoride; alternatively, a chloride; alternatively, a bromide; or alternatively, an iodide.

In an embodiment, each hydrocarbyl substituent which may be utilized as non-linking substituent on a Group I ligand, a hydrocarbyl group utilized in a linking group, or as a hydrocarbyl group within a non-linking substituent on a Group I ligand (e.g. trihydrocarbylsilyl group, N,N-dihydrocarbyl aminyl group, or hydrocarbylthiolate group, among others), can be independently an alkyl group, an alkenyl group, a cycloalkyl group, an aryl group, or an aralkyl group; alternatively, an alkyl group or an alkenyl group; alternatively, an alkyl group; alternatively, an alkenyl group; alternatively, a cycloalkyl group; alternatively, an aryl group; or alternatively, an aralkyl group. Generally, the alkyl, alkenyl, cycloalkyl, aryl, and aralkyl substituent groups can have the same number of carbon atoms as the hydrocarbyl substituent group disclosed herein.

In an embodiment, each alkyl substituent which may be utilized as non-linking substituent on a Group I ligand, an alkyl group utilized in a linking group, or as a alkyl group within a non-linking substituent on a Group I ligand (e.g. trihydrocarbylsilyl group, N,N-dihydrocarbyl aminyl group, or hydrocarbylthiolate group, among others), can be independently a methyl group, an ethyl group, an n-propyl group, an isopropyl group, an n-butyl group, a sec-butyl group, an isobutyl group, a tert-butyl group, an n-pentyl group, a 2-pentyl group, a 3-pentyl group, a 2-methyl-1-butyl group, a tert-pentyl group, a 3-methyl-1-butyl group, a 3-methyl-2-butyl group, a neo-pentyl group, a n-hexyl group, a n-heptyl group, or a n-octyl group; alternatively, a methyl group, an ethyl group, an n-propyl group, an isopropyl group, an n-butyl group, a sec-butyl group, an isobutyl group, a tert-butyl group, an n-pentyl group, a 2-pentyl group, a 3-pentyl group, a 2-methyl-1-butyl group, a tert-pentyl group, a 3-methyl-1-butyl group, a 3-methyl-2-butyl group, or a neo-pentyl group; alternatively, a methyl group, an ethyl group, an isopropyl group, a tert-butyl group, or a neo-pentyl group; alternatively, a methyl group; alternatively, an ethyl group; alternatively, an isopropyl group; alternatively, a tert-butyl group; alternatively, a neo-pentyl group; alternatively, an n-hexyl group; alternatively, an n-heptyl group; or alternatively, an n-octyl group.

In any embodiment disclosed herein, the Group I ligand, the Group II ligand, or both the Group I and Group II ligands can be substituted with a C₂ to C₂₀ alkenyl group; alternatively, a C₃ to C₁₅ alkenyl group; alternatively, a C₄ to C₁₀ alkenyl group; or alternatively, a C₄ to C₈ alkenyl group. Alternatively, in any embodiment disclosed herein, a substituent on a bridging atom of the linking group can be a C₂ to C₂₀ alkenyl group; alternatively, a C₃ to C₁₅ alkenyl group; alternatively, a C₄ to C₁₀ alkenyl group; or alternatively, a C₄ to C₈ alkenyl group. In any of these embodiments, and in one aspect the alkenyl groups can encompass “ω-alkenyl” groups, having their carbon-carbon double bond in the omega (ω)-position of the alkenyl moiety, that is, between the two carbon atoms furthest removed from the ligand to which the alkenyl group is bonded. Examples of ω-alkenyl groups include, but are not limited to, groups having the formula —CH₂(CH₂)ₙCH═CH₂, in which n can be an integer from 0 to 12; alternatively, n is an integer from 1 to 9; alternatively, n is an integer from 1 to 7; alternatively, n is an integer from 1 to 6; alternatively, n is an integer from 1 to 5; alternatively, n is an integer from 1 to 4; alternatively, n is an integer from 1 to 3; alternatively, n is an integer from 1 to 2. In a further aspect and in any embodiment, examples of ω-alkenyl groups include, but are not limited to, a group having the formula —CH₂(CH₂)ₘCH═CH₂, in which m is 0; alternatively, m is 1, alternatively, m is 2, alternatively, m is 3, alternatively, m is 4, alternatively, m is 5, alternatively, m is 6, alternatively, m is 7, alternatively, m is 8, alternatively, m is 9, alternatively, m is 10, alternatively, m is 11, or alternatively, m is 12. In an embodiment, any alkenyl substituent which may be utilized as non-linking substituent on a Group I ligand, an alkenyl group utilized in a linking group, or as a alkenyl group within non-linking substituent on a Group I ligand (e.g. trihydrocarbylsilyl group, N,N-dihydrocarbyl aminyl group, or hydrocarbylthiolate group, among others), can be an ethenyl group, a propenyl group, a butenyl group, pentenyl group, a hexenyl group; a heptenyl group, or an octenyl group; alternatively, a propenyl group, a butenyl group, pentenyl group, a hexenyl group; alternatively, an ethenyl group; alternatively, a propenyl group; alternatively, a butenyl group; alternatively, pentenyl group; alternatively, a hexenyl group; alternatively, heptenyl group; or alternatively, an octenyl group.

In an embodiment, any cycloalkyl substituent which may be utilized as non-linking substituent on a Group I ligand, a cycloalkyl group utilized in a linking group, or as a cycloalkyl group within non-linking substituent on a Group I ligand (e.g. trihydrocarbylsilyl group, N,N-dihydrocarbyl aminyl group, or hydrocarbylthiolate group, among others), can be a cyclopropyl group, a cyclobutyl group, a cyclopentyl group, a cyclohexyl group, a cycloheptyl group, or a cyclooctyl group; alternatively, a cyclopentyl group or a cyclohexyl group; alternatively, a cyclopropyl group; alternatively, a cyclobutyl group; alternatively, a cyclopentyl group; alternatively, a cyclohexyl group; alternatively, a cycloheptyl group; or alternatively, a cyclooctyl group. In an embodiment, any aryl substituent which may be utilized as non-linking substituent on a Group I ligand, an aryl group utilized in a linking group, or as an aryl group within non-linking substituent on a Group I ligand (e.g. trihydrocarbylsilyl group, N,N-dihydrocarbyl aminyl group, or hydrocarbylthiolate group, among others), can be phenyl group, a tolyl group, a xylyl group, or a 2,4,6-trimethylphenyl group; alternatively, a phenyl group; alternatively, a tolyl group, alternatively, a xylyl group; or alternatively, a 2,4,6-trimethylphenyl group. In an embodiment, any aralkyl substituent which may be utilized as non-linking substituent on a Group I ligand, an aralkyl group utilized in a linking group, or as a aralkyl group within non-linking substituent on a Group I ligand (e.g. trihydrocarbylsilyl group, N,N-dihydrocarbyl aminyl group, or hydrocarbylthiolate group, among others), can be a benzyl group.

In an embodiment, any hydrocarboxy substituent(s) which may be utilized as non-linking substituent on a Group I ligand can be an alkoxy group, an aroxy group, or an aralkoxy group; alternatively, an alkoxy group; alternatively, an aroxy group; or alternatively, an aralkoxy group. Generally, the alkoxy, aroxy, and aralkoxy substituent groups can have the same number of carbon atoms as the hydrocarboxy substituent group disclosed herein. In an embodiment, any alkoxy substituent which may be utilized as non-linking substituent on a Group I ligand can be a methoxy group, an ethoxy group, an n-propoxy group, an isopropoxy group, an n-butoxy group, a sec-butoxy group, an isobutoxy group, a tert-butoxy group, an n-pentoxy group, a 2-pentoxy group, a 3-pentoxy group, a 2-methyl-1-butoxy group, a tert-pentoxy group, a 3-methyl-1-butoxy group, a 3-methyl-2-butoxy group, or a neo-pentoxy group; alternatively, a methoxy group, an ethoxy group, an isopropoxy group, a tert-butoxy group, or a neo-pentoxy group; alternatively, a methoxy group; alternatively, an ethoxy group; alternatively, an isopropoxy group; alternatively, a tert-butoxy group; or alternatively, a neo-pentoxy group. In an embodiment, any aryl substituent which may be utilized as non-linking substituent on a Group I ligand can be a phenoxy group, a toloxy group, a xyloxy group, or a 2,4,6-trimethylphenoxy group; alternatively, a phenoxy group; alternatively, a toloxy group, alternatively, a xyloxy group; or alternatively, a 2,4,6-trimethylphenoxy group. In an embodiment, any aroxy substituent which may be utilized as non-linking substituent on a Group I ligand can be a benzoxy group.

Throughout this disclosure, metallocenes are described as comprising at least one Group II ligand. In this aspect and in any embodiment, the Group II ligands include those sigma-bonded ligands and some pi-bonded ligands in the metallocene that are not the η⁵-cycloalkadienyl-type ligands and are not the other pi-bonded η^{x≧5} ligands typically associated with defining a metallocene compound. In any embodiment disclosed herein, examples and alternative embodiments of Group II ligands include, but are not limited to, a hydride, a halide, a C₁-C₃₀ η^{x<5}-organic group, a C₁-C₃₀ η^{x<5}-hydrocarbon group, a C₁-C₃₀ aliphatic group, a C₆-C₃₀ η^{x<5}-aromatic group, a C₂-C₃₀ η^{x<5}-heterocyclic group, a C₂-C₃₀ η^{x<5}-cyclohetero group, a C₄-C₃₀ η^{x<5}-heteroarene group, a C₄-C₃₀ η^{x<5}-arylhetero group, a C₁-C₃₀ η^{x<5}-organohetero group, a C₅-C₃₀ heteroaralkane group, a C₅-C₂₀ heteroaralkane group, a C₅-C₁₀ heteroaralkane, a C₁-C₃₀ oxygen group, a C₁-C₃₀ sulfur group, a C₀-C₃₀ nitrogen group, a C₀-C₃₀ phosphorus group, a C₀-C₃₀ arsenic group, a C₀-C₃₀ silicon group, a C₀-C₃₀ germanium group, a C₀-C₃₀ tin group, a C₀-C₃₀ lead group, a C₀-C₃₀ boron group, or a C₀-C₃₀ aluminum group; alternatively, a hydride, a halide, a C₁-C₂₀ η^{x<5}-organic group, a C₁-C₂₀ η^{x<5}-hydrocarbon group, a C₁-C₂₀ aliphatic group, a C₆-C₂₀ η^{x<5}-aromatic group, a C₂-C₂₀ η^{x<5}-heterocyclic group, a C₂-C₂₀ η^{x<5}-cyclohetero group, a C₄-C₂₀ η^{x<5}-heteroarene group, a C₄-C₂₀ η^{x<5}-arylhetero group, a C₁-C₂₀ η^{x<5}-organohetero group, a C₅-C₂₀ heteroaralkane group, a C₁-C₂₀ oxygen group, a C₁-C₂₀ sulfur group, a C₀-C₂₀ nitrogen group, a C₀-C₂₀ phosphorus group, a C₀-C₂₀ arsenic group, a C₀-C₂₀ silicon group, a C₀-C₂₀ germanium group, a C₀-C₂₀ tin group, a C₀-C₂₀ lead group, a C₀-C₂₀ boron group, or a C₀-C₂₀ aluminum group; alternatively, a hydride, a halide, a C₁-C₁₀ η^{x<5}-organic group, a C₁-C₁₀ η^{x<5}-hydrocarbon group, a C₁-C₁₀ aliphatic group, a C₆-C₁₀ η^{x<5}-aromatic group, a C₂-C₁₀ η^{x<5}-heterocyclic group, a C₂-C₁₀ η^{x<5}-cyclohetero group, a C₄-C₁₀ heteroarene group, a C₄-C₁₀ η^{x<5}-arylhetero group, a C₁-C₁₀ η^{x<5}-organohetero group, a C₅-C₁₀ heteroaralkane, a C₁-C₁₀ oxygen group, a C₁-C₁₀ sulfur group, a C₀-C₁₀ nitrogen group, a C₀-C₁₀ phosphorus group, a C₀-C₁₀ arsenic group, a C₀-C₁₀ silicon group, a C₀-C₁₀ germanium group, a C₀-C₁₀ tin group, a C₀-C₅ tin group, a C₀-C₁₀ lead group, a C₀-C₁₀ boron group, or a C₀-C₁₀ aluminum group; alternatively, a hydride, a halide, a fluoride, a C₁-C₅ η^{x<5}-organic group, a C₁-C₅ η^{x<5}-hydrocarbon group, a C₁-C₅ aliphatic group, a C₆-C₁₀ η^{x<5}-aromatic group, a C₆-C₁₀ η^{x<5}-arene group, a C₂-C₅ η^{x<5}-heterocyclic group, a C₄-C₅ η^{x<5}-cyclohetero group, a C₄-C₅ heteroarene group, a C₄-C₅ η^{x<5}-arylhetero group, a C₁-C₅ η^{x<5}-organohetero group, a C₅-C₁₀ heteroaralkane, a C₁-C₅ oxygen group, a C₁-C₅ sulfur group, a C₀-C₅ nitrogen group, a C₀-C₅ phosphorus group, a C₀-C₅ arsenic group, a C₀-C₅ silicon group, a C₀-C₅ germanium group, a C₀-C₅ tin group, a C₀-C₅ lead group, a C₀-C₅ boron group, or a C₀-C₅ aluminum group.

Alternatively and in any embodiment of this disclosure, in each occurrence the Group II ligand can independently be a halide, a hydride, a C₁-C₃₀ η^{x<5}-hydrocarbyl group, a C₁-C₃₀ oxygen-bonded group, a C₁-C₃₀ sulfur-bonded group, a C₀-C₃₀ nitrogen-bonded group, a C₀-C₃₀ phosphorus-bonded group, a C₀ to C₂₀ arsenic-bonded group, a C₁-C₃₀ η^{x<5}-organyl group, a C₀-C₃₀ silicon-bonded group, a C₀-C₃₀ germanium-bonded group, a C₀-C₃₀ tin-bonded group, a C₀ to C₃₀ lead-bonded group, a C₀ to C₃₀boron-bonded group, a C₀ to C₃₀ aluminum-bonded group, or a C₀ to C₁₀ aluminum-bonded group; alternatively, a halide, a hydride, a C₁-C₂₀ η^{x<5}-hydrocarbyl group, a C₁-C₂₀ oxygen-bonded group, a C₁-C₂₀ sulfur-bonded group, a C₀-C₂₀ nitrogen-bonded group, a C₀-C₂₀ phosphorus-bonded group, a C₀ to C₂₀ arsenic-bonded group, a C₁-C₂₀ η^{x<5}-organyl group, a C₀-C₂₀ silicon-bonded group, a C₀-C₂₀ germanium-bonded group, a C₀-C₂₀ tin-bonded group, a C₀ to C₂₀ lead-bonded group, or a C₀ to C₂₀ aluminum-bonded group; alternatively, a halide, a hydride, a C₁-C₁₀ η^{x<5}-hydrocarbyl group, a C₁-C₁₀ oxygen-bonded group, a C₁-C₁₀ sulfur-bonded group, C₀-C₁₀ nitrogen-bonded group, a C₀-C₁₀ phosphorus-bonded group, a C₀ to C₁₀ arsenic-bonded group, a C₁-C₁₀ η^{x<5}-organyl group, a C₀-C₁₀ silicon-bonded group, a C₀-C₁₀ germanium-bonded group, a C₀-C₁₀ tin-bonded group, a C₀ to C₁₀ lead-bonded group, C₀ to C₁₀ boron-bonded group, or a C₀ to C₁₀ aluminum-bonded group; or alternatively, a halide, a hydride, a C₁-C₅ η^{x<5}-hydrocarbyl group, a C₁-C₁₀ oxygen-bonded group, a C₁-C₁₀ sulfur-bonded group, a C₀-C₁₀ nitrogen-bonded group, a C₀-C₁₀ phosphorus-bonded group, a C₀ to C₆ arsenic-bonded group, a C₁-C₅ η^{x<5}-organyl group, a C₀-C₁₀ silicon-bonded group, a C₀-C₁₀ germanium-bonded group, a C₀-C₁₀ tin-bonded group, a C₀ to C₁₀ lead-bonded group, a C₀ to C₁₀ boron-bonded group, or a C₀ to C₁₀ aluminum-bonded group.

In a further aspect and in any embodiment disclosed herein, any Group II ligand in each occurrence can include, but are not limited to, a halide, a hydride, a C₁-C₂₀ η^{x<5}-hydrocarbyl group, a C₁-C₂₀ oxygen-bonded group, a C₁-C₂₀ sulfur-bonded group, a C₀-C₃₀ nitrogen-bonded group, a C₀-C₂₀ phosphorus-bonded group, a C₁-C₂₀ η^{x<5}-organyl group, a C₀-C₃₀ silicon-bonded group, a C₁-C₃₀ germanium-bonded group, or a C₁-C₃₀ tin-bonded group; alternatively, a halide, a hydride, a C₁-C₁₀ η^{x<5}-hydrocarbyl group, a C₁-C₁₀ oxygen-bonded group, a C₁-C₁₀ sulfur-bonded group, a C₀-C₂₀ nitrogen-bonded group, a C₀-C₂₀ phosphorus-bonded group, a C₁-C₁₀ η^{x<5}-organyl group, a C₀-C₃₀ silicon-bonded group, a C₁-C₂₀ germanium-bonded group, or a C₁-C₂₀ tin-bonded group; or alternatively, a halide, a hydride, a C₁-C₅ η^{x<5}-hydrocarbyl group, a C₁-C₅ oxygen-bonded group, a C₁-C₅ sulfur-bonded group, a C₀-C₁₀ nitrogen-bonded group, a C₀-C₁₀ phosphorus-bonded group, a C₁-C₅ η^{x<5}-organyl group, a C₀-C₁₀ silicon-bonded group, a C₁-C₁₀ germanium-bonded group, or a C₁-C₁₀ tin-bonded group.

Yet a further aspect provides that, in any embodiment disclosed, any Group II ligand in each occurrence can independently be a halide, a hydride, a C₁-C₂₀ η^{x<5}-hydrocarbyl group, a C₁-C₂₀ oxygen-bonded group, a C₁-C₂₀ sulfur-bonded group, a C₀-C₃₀ nitrogen-bonded group, a C₁-C₂₀ η^{x<5}-organyl group, or a C₀-C₃₀ silicon-bonded group; alternatively, a halide, a hydride, a C₁-C₁₀ η^{x<5}-hydrocarbyl group, a C₁-C₁₀ oxygen-bonded group, a C₁-C₁₀ sulfur-bonded group, a C₀-C₂₀ nitrogen-bonded group, a C₀-C₂₀ phosphorus-bonded group, a C₁-C₁₀ η^{x<5}-organyl group, or a C₀-C₂₀ silicon-bonded group; or alternatively, a halide, a hydride, a C₁-C₅ η^{x<5}-hydrocarbyl group, a C₁-C₅ oxygen-bonded group, a C₁-C₅ sulfur-bonded group, a C₀-C₁₀ phosphorus-bonded group, a C₁-C₅ η^{x<5}-organyl group, or a C₀-C₁₀ silicon-bonded group.

Alternatively, and any embodiment, in each occurrence the Group II ligand can independently be a halide, a hydride, a C₁ to C₂₀ hydrocarboxide group (also referred to as a hydrocarboxy group), a C₁ to C₂₀ heterocyclic group, a C₆ to C₂₀ η¹-aromatic group, a C₁ to C₂₀ η¹-heteroaromatic group, a C₁ to C₂₀ hydrocarbylsilyl group, a C₁ to C₂₀ dihydrocarbylsilyl group, a C₁ to C₂₀ trihydrocarbylsilyl group, an aminyl group, an C₁ to C₂₀ N-hydrocarbylaminyl group, a C₁ to C₂₀ N,N-dihydrocarbylaminyl group, a C₁ to C₂₀ hydrocarbylthiolate group, or a C₃ to C₃₀ trihydrocarbylsiloxy group. In a further alternative and in each occurrence, the Group II ligand can independently be a halide, a hydride, a C₁ to C₂₀ alkoxide, a C₆ to C₂₀ aryloxide, a C₆ to C₂₀ η¹-aromatic group, an amido group, a C₁ to C₂₀ N-alkylamido group, a C₆ to C₂₀ N-arylamido group, C₁ to C₂₀ N,N-dialkylamido group, a C₇ to C₂₀ N-alkyl-N-arylamido group, a C₁ to C₂₀ alkylthiolate, a C₆ to C₂₀ arylthiolate, a C₃ to C₂₀ trialkylsiloxy, or a C₁₈ to C₃₀ triarylsiloxy.

In one additional aspect, and in any embodiment, in each occurrence the Group II ligand can independently be a halide, a C₁ to C₂₀ hydrocarboxide (also referred to as a hydrocarboxy group), a C₁ to C₃₀ hydrocarbyl, or a C₃ to C₂₀ trihydrocarbylsiloxy; alternatively, a halide, a C₁ to C₁₀ hydrocarboxide, a C₁ to C₁₀ hydrocarbyl, or a C₃ to C₂₀ trihydrocarbylsiloxy; or alternatively, a halide, a C₁ to C₅ hydrocarboxide, a C₁ to C₅ hydrocarbyl, or a C₃ to C₁₅ trihydrocarbylsiloxy. In another aspect, and in any embodiment, in each occurrence the Group II ligand can independently be a halide, a C₁ to C₂₀ hydrocarboxide, or a C₁ to C₃₀ hydrocarbyl; alternatively, a halide, a C₁ to C₁₀ hydrocarboxide, or a C₁ to C₁₀ hydrocarbyl; or alternatively, a halide, a C₁ to C₅ hydrocarboxide, or a C₁ to C₅ hydrocarbyl. In another aspect, and in any embodiment, in each occurrence the Group II ligand can independently be a halide or a C₁ to C₂₀ hydrocarboxide; alternatively, a halide or a C₁ to C₁₀ hydrocarboxide; or alternatively, a halide or a C₁ to C₅ hydrocarbyl. In a further aspect, in each occurrence the Group II ligand can be a halide.

Halides have been disclosed herein as potential non-linking substituents on a Group I ligand or as a halide utilized in a linking group and these halide may be utilized, without limitation and in any aspect or embodiment, as a Group II ligand. Hydrocarbyl groups have been disclosed herein as potential non-linking substituent on a Group I ligand, a hydrocarbyl group utilized in a linking group, or as a hydrocarbyl group within a non-linking substituent on a Group I ligand and these hydrocarbyl groups can be utilized, without limitation and in any aspect or embodiment, as a Group II ligand. Hydrocarboxy groups have been disclosed herein as potential non-linking substituent on a Group I ligand and these hydrocarboxy groups can be utilized, without limitation and in any aspect or embodiment, as a Group II ligand.

Substituted aminyl groups which may be utilized in any embodiment calling for a substituted aminyl group can may be an N-hydrocarbyl aminyl group or an N,N-dihydrocarbyl aminyl group. Hydrocarbyl groups have been described herein and these hydrocarbyl groups can be utilized, without limitation, to further described the N-hydrocarbyl aminyl group or an N,N-dihydrocarbyl aminyl group which may be utilized in various aspects and embodiments described herein. In a non-limiting embodiment, N-hydrocarbyl aminyl groups which may be utilized in any embodiment calling for a N-hydrocarbyl aminyl group include, but are not limited to, N-methylaminyl group (—NHCH₃), a N-ethylaminyl group (—NHCH₂CH₃), a N-n-propylaminyl group (—NHCH₂CH₂CH₃), an N-iso-propylaminyl group (—NHCH(CH₃)₂), a N-n-butylaminyl group (—NHCH₂CH₂CH₂CH₃), a N-t-butylaminyl group (—NHC(CH₃)₃), a N-n-pentylaminyl group (—NHCH₂CH₂CH₂CH₂CH₃), a N-neo-pentylaminyl group (—NHCH₂C(CH₃)₃), a N-phenylaminyl group (—NHC₆H₅), a N-tolylaminyl group (—NHC₆H₄—CH₃), or a N-xylylaminyl group (—NHC₆H₃(CH₃)₂); alternatively, a N-ethylaminyl group; alternatively, a N-propylaminyl group; or alternatively, a N-phenylaminyl group. A N,N-dihydrocarbyl aminyl group which may be utilized in any embodiment caring for a N,N-dihydrocarbylaminyl groups include, but are not limited to a N,N-dimethylaminyl group (—N(CH₃)₂), a N,N-diethylaminyl group (—N(CH₂CH₃)₂), a N,N-di-n-propylaminyl group (—N(CH₂CH₂CH₃)₂), a N,N-di-iso-propylaminyl group (—N(CH(CH₃)₂)₂), a N,N-di-n-butylaminyl group (—N(CH₂CH₂CH₂CH₃)₂), a N,N-di-t-butylaminyl group (—N(C(CH₃)₃)₂), a N,N-di-n-pentylaminyl group (—N(CH₂CH₂CH₂CH₂CH₃)₂), a N,N-di-neo-pentylaminyl group (—N(CH₂C(CH₃)₃)₂), a N,N-di-phenylaminyl group (—N(C₆H₅)₂), a N,N-di-tolylaminyl group (—N(C₆H₄—CH₃)₂), or a N,N-di-xylylaminyl group (—N(C₆H₃(CH₃)₂)₂); alternatively, a N,N-di-ethylaminyl group; alternatively, a N,N-di-n-propylaminyl group; or alternatively, a N,N-di-phenylaminyl group. Halides which may be utilized in any embodiment caring for a halide substituent or group includes fluoride, chloride, bromide, or iodide; alternatively, fluoride; alternatively, chloride; or alternatively, bromide. In some embodiments, substituents or groups which may be utilized in an embodiment calling for a substituent or group can include a halogenated hydrocarbyl group. In an embodiment, the halogenated hydrocarbyl group can be a halogenated aromatic group or a halogenated alkyl group; alternatively, a halogenated aromatic group; or alternatively, a halogenated alkyl group. One popular halogenated aromatic group is pentafluorophenyl. One popular halogenated alky group is trifluoromethyl.

Examples of aromatic groups, in each instance, include, but are not limited to, phenyl, naphthyl, anthracenyl, and the like, including substituted derivatives thereof. In some embodiments, the aromatic group can be a substituted phenyl groups. The substituted phenyl group can be substituted at the 2 position, the 3 position, the 4 position, the 2 and 4 positions, the 2 and 6 positions, the 2 and 5 positions, the 3 and 5 positions, or the 2, 4, and 6 positions; alternatively, the 2 position, the 4 position, the 2 and 4 positions, the 2 and 6 positions, or the 2, 4, and 6 positions; alternatively, 2 position; alternatively, the 3 position; alternatively, the 4 position; alternatively, the 2 and 4 positions; alternatively, the 2 and 6 positions; alternatively, the 3 and 5 positions; or alternatively, the 2, 4, and 6 positions. Substituents which can be present included a halide, an alkyl group, an alkoxy group, an aminyl group, an N-hydrocarbylaminyl, and/or a N,N-dihydrocarbylaminyl group; alternatively, a halide, an alkyl group, or an alkoxy group; alternatively, a halide or an alkyl group; alternatively, a halide or an alkoxy group; alternatively, a halide; alternatively, an alkyl group; or alternatively, an alkoxy group. Halides, alkyl groups, and alkoxy group have been independently described herein and can be utilized, without limitation as each independent substituent. Some non-limiting embodiments, substituted aromatic groups include, but are not limited to, tolyl (2-, 3-, 4-, or mixtures thereof), xylyl (2,3-, 2,4-, 2,5-, 3,4-, 3,5-, 2,6-, or mixtures thereof), mesityl, pentafluorophenyl, C₆H₄OMe (2-, 3-, 4-, or mixtures thereof), C₆H₄NH₂ (2-, 3-, 4-, or mixtures thereof), C₆H₄NMe₂ (2-, 3-, 4-, or mixtures thereof), C₆H₄CF₃ (2-, 3-, 4-, or mixtures thereof), C₆H₄F, C₆H₄Cl (2-, 3-, 4-, or mixtures thereof), C₆H₃(OMe)₂ (2,3-, 2,4-, 2,5-, 3,4-, 3,5-, 2,6-, or mixtures thereof), C₆H₃(CF₃)₂ (2,3-, 2,4-, 2,5-, 3,4-, 3,5-, 2,6-, or mixtures thereof), and the like, including any heteroatom substituted analogs thereof as described in the definitions section. Other substituted aromatic groups, and combinations of substituted aromatic groups, can be envisioned utilizing the present disclosure.

Examples of heterocyclic compounds from which heteroatom groups can be derived include, but are not limited to, aziridine, azirine, oxirane (ethylene oxide), oxirene, thiirane (ethylene sulfide), dioxirane, azetidine, oxetane, thietane, dioxetane, dithietane, tetrahydropyrrole, pyrrole, tetrahydrofuran, furan, tetrahydrothiophene, thiophene, imidazolidine, pyrazole, imidazole, oxazolidine, oxazole, isoxazole, thiazolidine, thiazole, isothiazole, dioxolane, dithiolane, triazoles, dithiazole, tetrazole, piperidine, pyridine, tetrahydropyran, pyran, thiane, thiine, piperazine, diazines, oxazines, thiazines, dithiane, dioxane, dioxin, triazine, trioxane, tetrazine, azepine, thiepin, diazepine, morpholine, quinoline, 1,2-thiazole, bicyclo[3.3.1]tetrasiloxane, and their substituted analogs. Accordingly and as applicable to the particular heterocyclic compound, heterocyclyl groups, heterocyclylene groups, heterocyclic groups, cycloheteryl groups, cycloheterylene groups, cyclohetero groups, heteroaryl groups, heteroarylene groups, heteroarene groups, arylheteryl groups, arylheterylene groups, arylhetero groups, organoheteryl groups, organoheterylene groups, or organohetero groups can be derived from these and similar heterocyclic compounds and their substituted analogs. Additional description is provided in the definitions section.

In a further aspect, and in any embodiment disclosed herein in which ligands are selected to impart optical activity to the metallocene, the metallocene can be racemic. Alternatively, and in any embodiment in which ligands are selected to impart optical activity to the metallocene, the metallocene can be non-racemic. Further, and in any embodiment in which ligands are selected to impart optical activity to the metallocene, the metallocene can be substantially optically pure (having an enantiomeric excess of greater than or equal to 99.5%), or not optically pure. Thus, any enantiomer, diastereomer, epimer, and the like of the metallocene used in the methods described herein are encompassed by this disclosure.

In another aspect and in any embodiment disclosed herein, the metallocene can have the formula (η⁵-cycloalkadienyl)M³R⁹ₙX⁹₃₋ₙ; or alternatively, have the formula (η⁵-cycloalkadienyl)₂M³X⁹₂. In an embodiment, M³ can be any metallocene metal described herein each η⁵-cycloalkadienyl ligand can be independently any η⁵-cycloalkadienyl ligand described herein, each R⁹ can be independently any hydrocarbyl group described herein, each X⁹ can be independently any halide, hydrocarbyl group, hydrocarboxy group described herein, and can be an integer from 1 to 3. In some non-limiting embodiments, M³ can be Ti, Zr, or Hf, each η⁵-cycloalkadienyl ligand can be a substituted or an unsubstituted cyclopentadienyl ligand, a substituted or an unsubstituted indenyl ligand, or a substituted or an unsubstituted fluorenyl ligand, each R⁹ can be independently a substituted or an unsubstituted C₁-C₂₀ alkyl group, C₁-C₂₀ cycloalkyl group, C₆-C₂₀ aryl group, or C₇-C₂₀ aralkyl group, each X⁹ can be independently a halide, a substituted or an unsubstituted C₁-C₂₀ alkyl group, a substituted or an unsubstituted C₁-C₂₀ cycloalkyl group, a substituted or an unsubstituted C₆-C₂₀ aryl group, a substituted or an unsubstituted C₇-C₂₀ aralkyl group, a substituted or an unsubstituted C₁-C₂₀ alkoxide group, or a substituted or an unsubstituted C₆-C₂₀ aryloxide group, and n can be an integer from 1 to 3. When the metallocene has the formula (η⁵-cycloalkadienyl)₂M³X⁹₂ the two (η⁵-cycloalkadienyl) ligand can be linked by any linking group described herein. When the metallocene having the formula (η⁵-cycloalkadienyl)M³R⁹ₙX⁹₃₋ₙ or the formula (η⁵-cycloalkadienyl)₂M³X⁹₂, any non-linking substituent on the (η⁵-cycloalkadienyl, R⁹, and/or X⁹ may independently be any substituent group disclosed herein. In some embodiments, when the metallocene having the formula (η⁵-cycloalkadienyl)M³R⁹ₙX⁹₃₋ₙ or the formula (η⁵-cycloalkadienyl)₂M³X⁹₂, any non-linking substituent on the η⁵-cycloalkadienyl, R⁹, and/or X⁹ may independently be a halide, a C₁ to C₂₀ alkoxide group, a C₆ to C₂₀ aryloxide group, a C₆ to C₂₀ aromatic group, an amido group, a C₁ to C₂₀ N-alkylamido group, a C₆ to C₂₀ N-arylamido group, C₁ to C₄₀ N,N-dialkylamido group, a C₇ to C₄₀ N-alkyl-N-arylamido group, a C₁ to C₂₀ alkylthiolate group, a C₀ to C₂₀ arylthiolate group, a C₃ to C₂₀ trialkylsiloxy group, or a C₁₈ to C₄₅ triarylsiloxy group.

A wide range of metallocenes are useful in the catalyst systems disclosed herein and/or the practice of the methods disclosed herein. In an aspect and in any embodiment disclosed herein, the metallocene can have the formula:

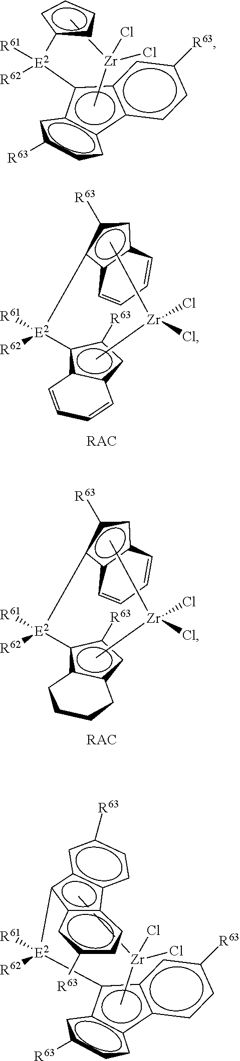

or any combination thereof. In an aspect and in any embodiment disclosed herein, the metallocenecan have the formula:

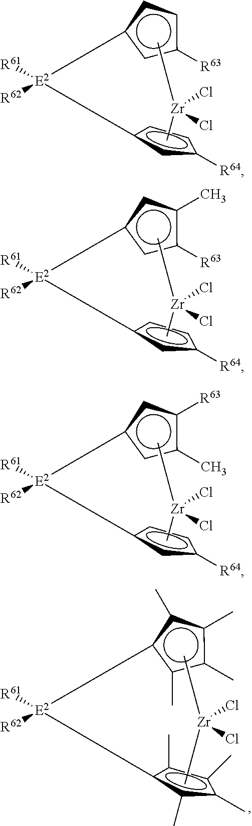

or any combination thereof. In this aspect, E² can be any bridging atom disclosed herein, and R⁶¹, R⁶², R⁶³, and R⁶⁴ in each occurrence can be independently any hydrocarbyl group disclosed herein. In some non-limiting embodiments, E² can be C, Si, Ge, or Sn, and in each occurrence, R⁶¹, R⁶², R⁶³, and R⁶⁴ can be independently H or any C₁-C₂₀ hydrocarbyl group described herein.

In another non-limiting aspect and in any embodiment disclosed herein, the metallocene can have the formula:

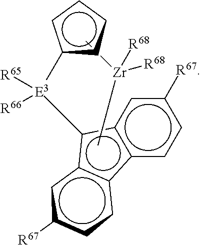

In this aspect, E³ can be any bridging atom disclosed herein, R⁶⁵ can be H or any hydrocarbyl group disclosed herein, R⁶⁶ can be any alkenyl group disclosed herein, R⁶⁷ can be H or any hydrocarbyl group disclosed herein, and R⁶⁸ can be H or any hydrocarbyl group disclosed herein. In some non-limiting embodiments, E³ can be C, Si, Ge, or Sn, R⁶⁵ can be H or a C₁-C₂₀ hydrocarbyl group, R⁶⁶ can be a C₃-C₁₂ alkenyl group, R⁶⁷ can be H or a C₁-C₁₅ hydrocarbyl group, and R⁶⁸ can be H or a C₁-C₂₀ hydrocarbyl group.

In yet another aspect and in any embodiment disclosed herein, the metallocene can comprise, consist essentially of, or consist of, singly or in any combination:

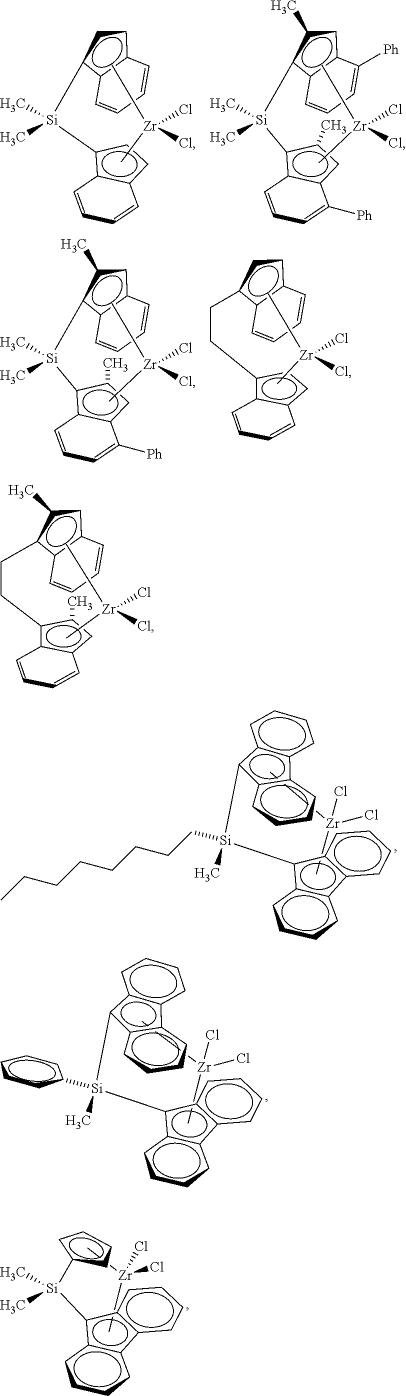

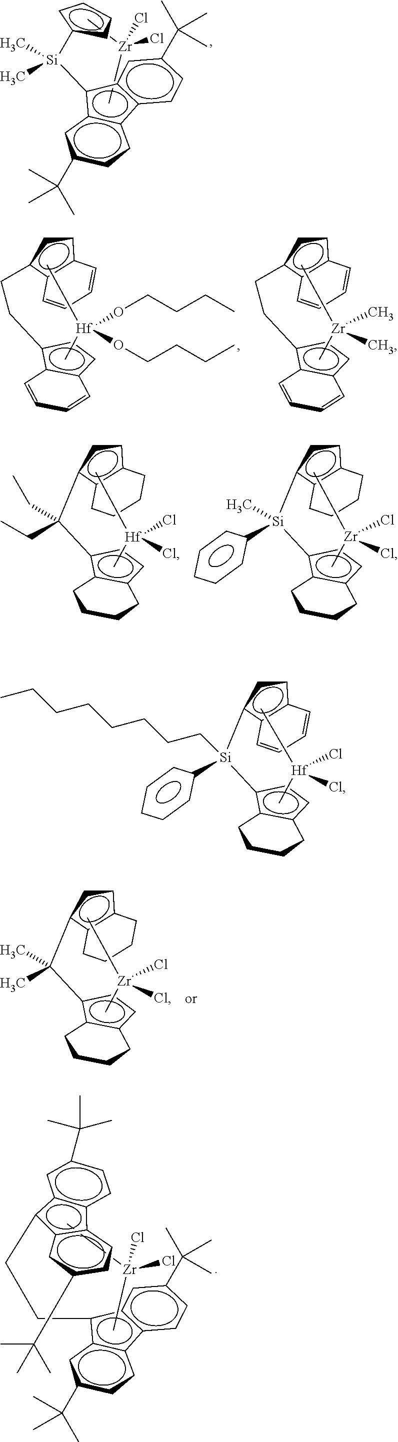

Still a further aspect and in any embodiment disclosed herein, the metallocene can comprise, consist essentially of, or consist of, singly or in any combination:

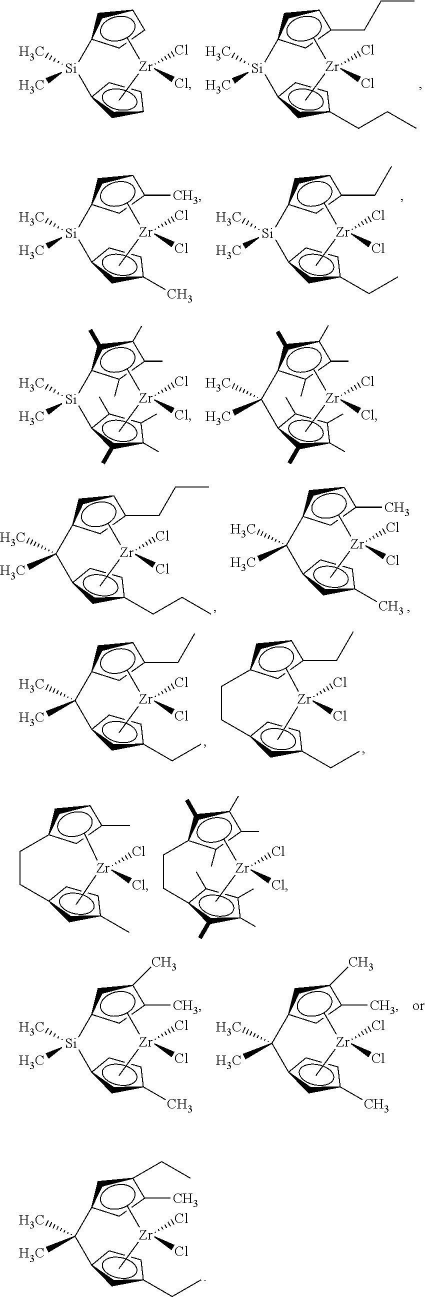

An additional aspect and in any embodiment of this disclosure, the metallocene can comprise, consist essentially of, or consist of, singly or in any combination:

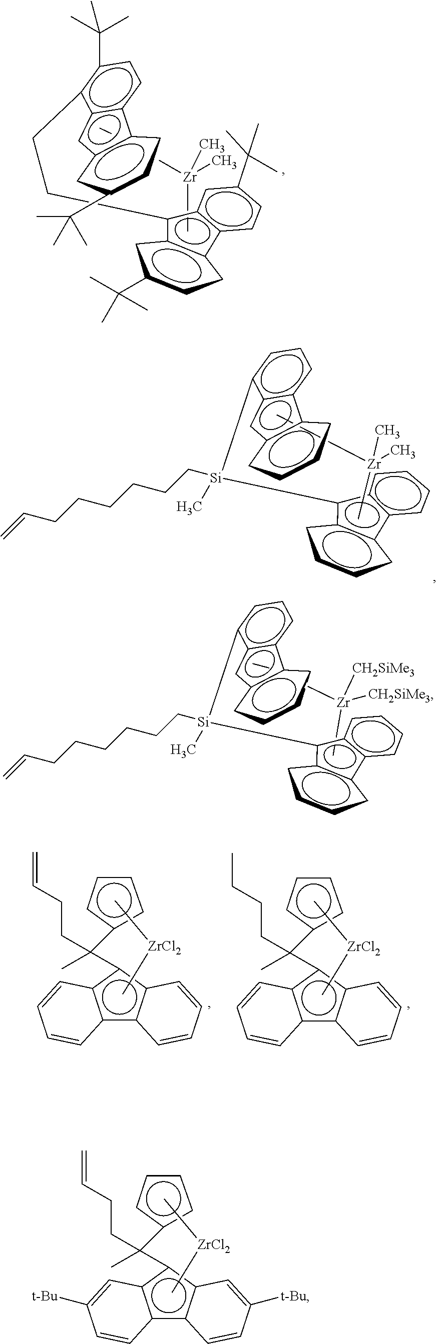

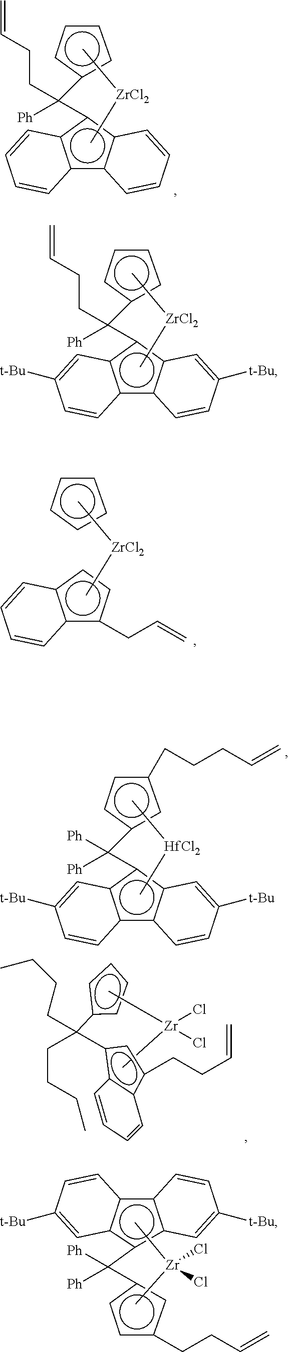

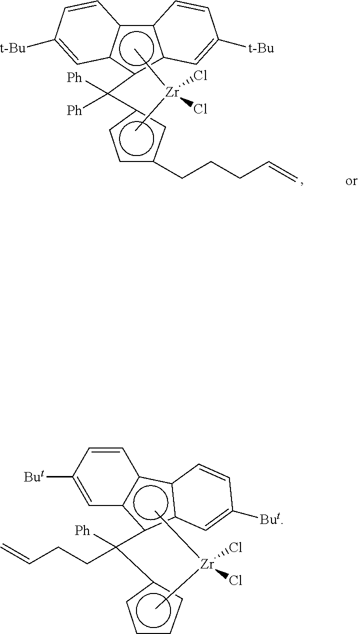

Another aspect an any embodiment disclosed herein, the metallocene can comprise, consist essentially of, or consist of, singly or in any combination:

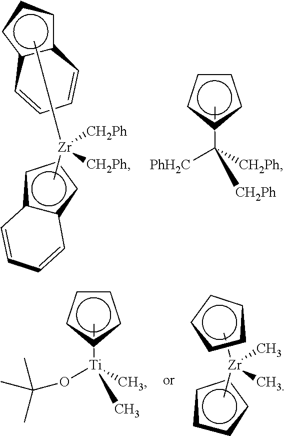

According to another aspect and in any embodiment disclosed herein, the metallocene can comprise, consist essentially of, or consist of:

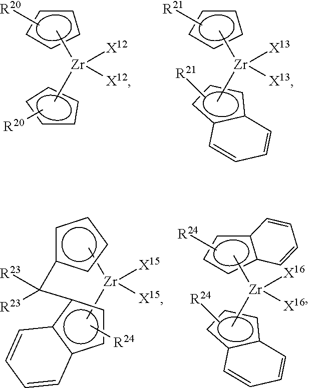

or combinations thereof; alternatively,

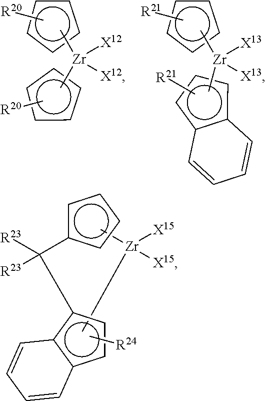

or combinations thereof; alternatively,

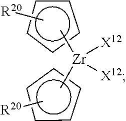

alternatively,

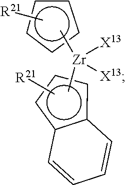

alternatively,

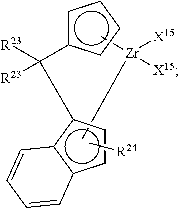

or alternatively,

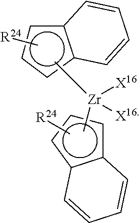

According to yet another aspect and in any embodiment disclosed herein, the metallocene can comprise, consist essentially of, or consist of:

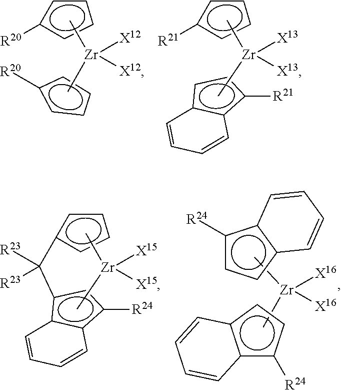

or any combinations thereof; alternatively,

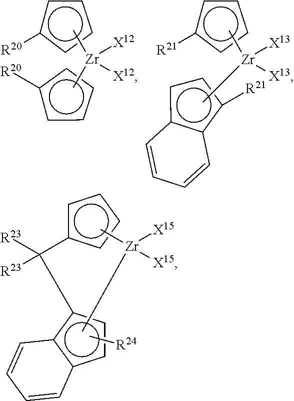

or any combination thereof; alternatively,

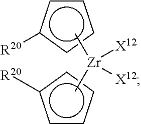

alternatively,

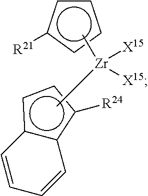

alternatively or

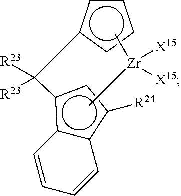

alternatively,

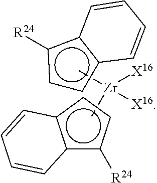

In these aspects, each R²⁰, R²¹, R²³, and R²⁴ can be independently hydrogen or any hydrocarbyl group disclosed herein, and each X¹², X¹³, X¹⁵, and X¹⁶ can be independently any halide described herein. In some embodiments, each R²⁰, R²¹, R²³, and R²⁴ can be independently a hydrogen, a C₁ to C₂₀ alkyl group, or a C₁ to C₂₀ alkenyl group, and each X¹², X¹³, X¹⁵, and X¹⁶ can be independently F, Cl, Br, or I. In other embodiments, each R²⁰, R²¹, and R²³ can be independently a hydrogen, a C₁ to C₁₀ alkyl group, or a C₁ to C₁₀ alkenyl group, and each X¹², X¹³, and X¹⁵ can independently be Cl or Br.

According to yet a further aspect and any embodiment disclosed herein, the metallocene can comprise, consist essentially of, or consist of:

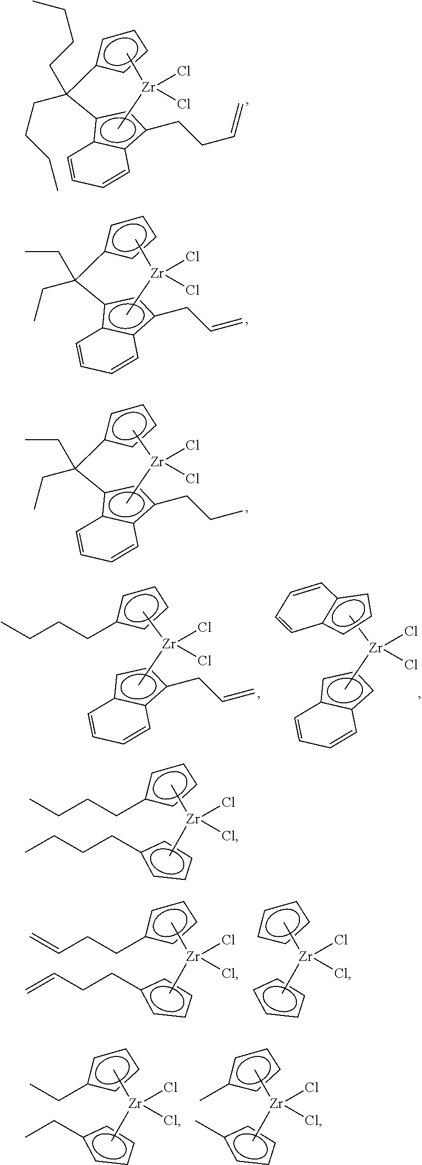

or any combination thereof; alternatively,

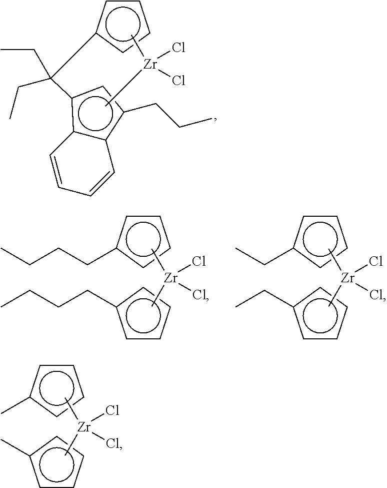

or any combination thereof; alternatively,

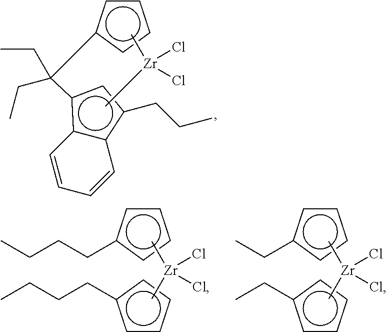

or any combination thereof; alternatively,

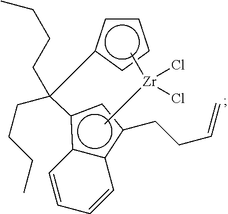

alternatively,

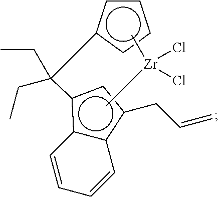

alternatively,

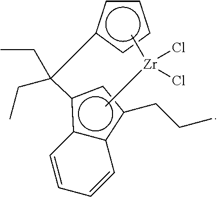

alternatively,

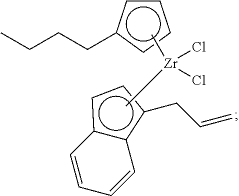

alternatively,

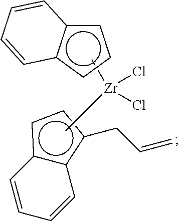

alternatively,

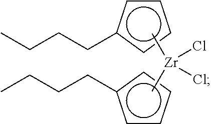

alternatively,

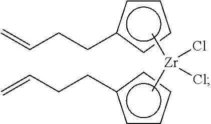

alternatively,

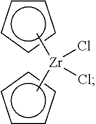

or alternatively,

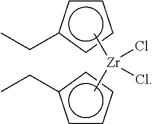

In another aspect and in any embodiment disclosed herein, the metallocene can comprise, consist essentially of, or consist of:

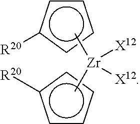

In an embodiment, each R²⁰ can be independently a hydrogen, a C₁ to C₁₀ alkyl group, or a C₁ to C₁₀ alkenyl group, and each X¹² can be independently Cl or Br. In other embodiments, each R²⁰ can be independently a C₁ to C₁₀ alkyl group and each X¹² can be independently Cl or Br. In a non-limiting embodiment, the metallocene can comprise, consist essentially of, or consists of:

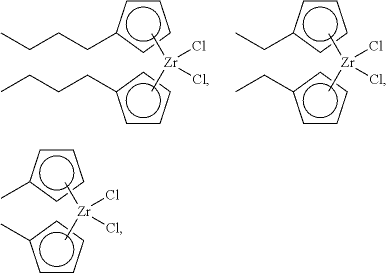

or any combination thereof; alternatively,

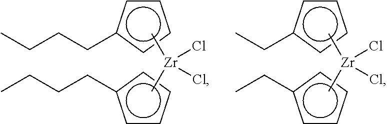

or any combination thereof.

Still a further aspect and any embodiment disclosed herein, the metallocene can comprise, consist essentially of, or consists of:

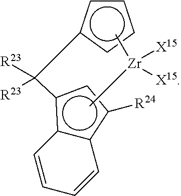

In some non-limiting embodiments, each R²³ and R²⁴ can be independently hydrogen, a C₁ to C₁₀ alkyl group, or a C₁ to C₁₀ alkenyl group, and each X¹⁵ can be independently Cl or Br. In other non-limiting embodiments, each R²³ and R²⁴ can be independently a C₁ to C₁₀ alkyl group or a C₁ to C₁₀ alkenyl group, and each X¹⁵ can be independently Cl or Br. In yet another non-limiting embodiment, the metallocene can comprise, consist essentially of, or consist of:

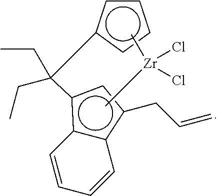

Still a further aspect and any embodiment disclosed herein, the metallocene can comprise, consist essentially of, or consists of:

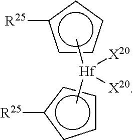

In some non-limiting embodiments, each R²⁵ can be independently hydrogen, a C₁ to C₁₀ alkyl group, or a C₁ to C₁₀ alkenyl group, and each X²⁰ can be independently Cl or Br. In other non-limiting embodiments, each R²⁵ can be independently a C₁ to C₁₀ alkyl group or a C₁ to C₁₀ alkenyl group, and each X²⁰ can be independently Cl or Br. In yet other non-limiting embodiments, each R²⁵ can be independently a C₁ to C₁₀ alkyl group, and each X²⁰ can be independently Cl or Br. In yet another non-limiting embodiment, the metallocene can comprise, consist essentially of, or consist of:

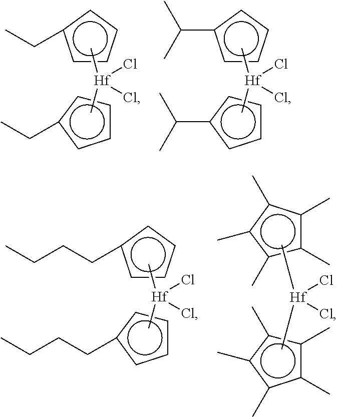

or combinations thereof; alternatively,

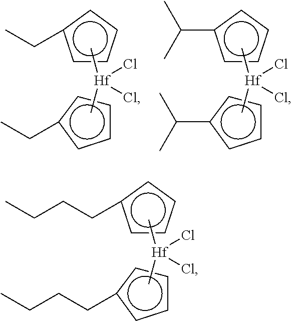

or combinations thereof; alternatively,

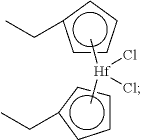

alternatively,

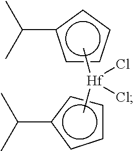

alternatively,

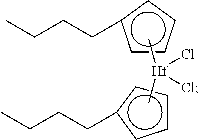

or alternatively,

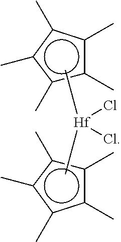

In other aspect and in any embodiment disclosed herein, the metallocene can comprise, consist essentially of, or consist of, singly or in any combination thereof:
bis(cyclopentadienyl)hafnium dichloride,
bis(cyclopentadienyl)zirconium dichloride,
1,2-ethanediylbis(η⁵-1-indenyl)di-n-butoxyhafnium,
1,2-ethanediylbis(η⁵-1-indenyl)dimethylzirconium,
3,3-pentanediylbis(η⁵-4,5,6,7-tetrahydro-1-indenyl)hafnium dichloride,
methylphenylsilylbis(η⁵-4,5,6,7-tetrahydro-1-indenyl)zirconium dichloride,
bis(n-butylcyclopentadienyl)di-t-butylamido hafnium,
bis(n-butylcyclopentadienyl)zirconium dichloride,
bis(ethylcyclopentadienyl)zirconium dichloride,
bis(propylcyclopentadienyl)zirconium dichloride,
dimethylsilylbis(1-indenyl)zirconium dichloride,
nonyl(phenyl)silylbis(1-indenyl)hafnium dichloride,
dimethylsilylbis(η⁵-4,5,6,7-tetrahydro-1-indenyl)zirconium dichloride,
dimethylsilylbis(2-methyl-1-indenyl)zirconium dichloride,
1,2-ethanediylbis(9-fluorenyl)zirconium dichloride, indenyl diethoxy titanium(IV) chloride,
(isopropylamidodimethylsilyl)cyclopentadienyltitanium dichloride,
bis(pentamethylcyclopentadienyl)zirconium dichloride,
bis(indenyl)zirconium dichloride,
methyloctylsilyl bis(9-fluorenyl) zirconium dichloride,
bis-[1-(N,N-diisopropylamino)boratabenzene]hydridozirconium trifluoromethylsulfonate,
bis(cyclopentadienyl)hafnium dimethyl,
bis(cyclopentadienyl)zirconium dibenzyl,
1,2-ethanediylbis(η⁵-1-indenyl)dimethylhafnium,
1,2-ethanediylbis(η⁵-1-indenyl)dimethylzirconium,
3,3-pentanediylbis(η⁵-4,5,6,7-tetrahydro-1-indenyl)hafnium dimethyl,
methylphenylsilylbis(η⁵-4,5,6,7-tetrahydro-1-indenyl)zirconium dimethyl,
bis(1-n-butyl-3-methyl-cyclopentadienyl)zirconium dimethyl,
bis(n-butylcyclopentadienyl)zirconium dimethyl,
dimethylsilylbis(1-indenyl)zirconium bis(trimethylsilylmethyl),
octyl(phenyl)silylbis(1-indenyl)hafnium dimethyl,
dimethylsilylbis(η⁵-4,5,6,7-tetrahydro-1-indenyl)zirconium dimethyl,
dimethylsilylbis(2-methyl-1-indenyl)zirconium dibenzyl,
1,2-ethanediylbis(9-fluorenyl)zirconium dimethyl,
(indenyl)trisbenzyl titanium(IV),
(isopropylamidodimethylsilyl)cyclopentadienyltitanium dibenzyl,
bis(pentamethylcyclopentadienyl)zirconium dimethyl,
bis(indenyl)zirconium dimethyl,
methyl(octyl)silylbis(9-fluorenyl)zirconium dimethyl,
bis(2,7-di-tert-butylfluorenyl)-ethan-1,2-diyl)zirconium(IV) dimethyl,
2-(η⁵-cyclopentadienyl)-2-η⁵-fluoren-9-yl)hex-5-ene zirconium(IV) dichloride,
2-(η⁵-cyclopentadienyl)-2-(η⁵-2,7-di-tert-butylfluoren-9-yl)hex-5-ene zirconium(IV) dichloride,
2-(η⁵-cyclopentadienyl)-2-(η⁵-fluoren-9-yl)hept-6-ene zirconium(IV) dichloride,
2-(η⁵-cyclopentadienyl)-2-(η⁵-2,7-di-tert-butylfluoren-9-yl)hept-6-ene zirconium(IV) dichloride,
1-(η⁵-cyclopentadienyl)-1-(η⁵-fluoren-9-yl)-1-phenylpent-4-ene zirconium(IV) dichloride,
1-(η⁵-cyclopentadienyl)-1-(η⁵-2,7-di-tert-butyl fluoren-9-yl)-1-phenylpent-4-ene zirconium(IV) dichloride,
1-(η⁵-cyclopentadienyl)-1-(η⁵-fluoren-9-yl)-1-phenylhex-5-ene zirconium(IV) dichloride, or
1-(η⁵-cyclopentadienyl)-1-(η⁵-2,7-di-tert-butylfluoren-9-yl)-1-phenylhex-5-ene zirconium(IV) dichloride.

In another aspect and in any embodiment disclosed herein, the metallocene can comprise, consist essentially of, consist of, singly or in any combination, rac-C₂H₄(η⁵-indenyl)₂ZrCl₂, rac-Me₂Si(η⁵-indenyl)₂ZrCl₂, Me(octyl)Si(η⁵-fluorenyl)₂ZrCl₂, rac-Me₂Si(η⁵-2-Me-4-Ph-indenyl)₂ZrCl₂, rac-C₂H₄(η⁵-2-Me-indenyl)₂ZrCl₂, Me(Ph)Si(re-fluorenyl)₂ZrCl₂, rac-Me₂Si(η⁵-3-n-Pr-cyclopentadienyl)₂ZrCl₂, Me₂Si(η⁵-Me₄-cyclopentadienyl)₂ZrCl₂, or Me₂Si(η⁵-cyclopentadienyl)₂ZrCl₂.

In still another aspect and in any embodiment disclosed herein, the metallocene can comprise, consist essentially of, or consists of a compound having the formula ZrR¹²R¹³R¹⁴X⁹₂ wherein each X⁹ independently is a halogen atom, R¹² is a neutral ether group, R¹³ is a η¹-aminyl group, R¹⁴ is a substituted or unsubstituted η¹-fluorenyl group, and wherein R¹³ and R¹⁴ are connected by a linking group.

In this aspect, for example, and in a non-limiting embodiment, the metallocene of formula ZrR¹²R¹³R¹⁴X⁹₂ may have the formula

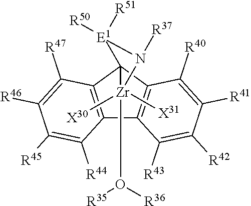

wherein E¹ can be C, Si, Ge, or Sn; R⁴⁰, R⁴¹, R⁴², R⁴³, R⁴⁴, R⁴⁵, R⁴⁶, and R⁴⁷ independently can be hydrogen or a C₁ to C₂₀ hydrocarbyl group (saturated or unsaturated); R⁵⁰ and R⁵¹ can be independently selected from a hydrogen, and saturated or unsaturated C₁-C₂₀ hydrocarbyl group; R³⁷ is a C₁-C₂₀ hydrocarbyl group; and R³⁵OR³⁶ represents an ether group wherein R³⁵ and R³⁶ independently can be a C₁-C₂₀ hydrocarbyl group. In an embodiment, E¹ can be C or Si; alternatively, C; or alternatively Si. In an embodiment, R⁴⁰, R⁴¹, R⁴², R⁴³, R⁴⁴, R⁴⁵, R⁴⁶, and R⁴⁷ independently can be hydrogen or a C₁-C₁₀ hydrocarbyl group; alternatively, hydrogen or a C₁-C₂₀ alkyl group; alternatively, hydrogen, or a C₁-C₁₀ alkyl group; or alternatively, hydrogen or a C₁-C₅ alkyl group. In other embodiments, R⁴⁰, R⁴³, R⁴⁴, and R⁴⁷ can be hydrogen and R⁴¹, R⁴², R⁴⁵, and R⁴⁶ independently can be hydrogen or a C₁ to C₂₀ hydrocarbyl groups; alternatively, R⁴⁰, R⁴³, R⁴⁴, and R⁴⁷ can be hydrogen and R⁴¹, R⁴², R⁴⁵, and R⁴⁶ independently can be hydrogen or a C₁-C₁₀ hydrocarbyl group; alternatively, R⁴⁰, R⁴³, R⁴⁴, and R⁴⁷ can be hydrogen and R⁴¹, R⁴², R⁴⁵, and R⁴⁶ independently can be hydrogen or a C₁-C₂₀ alkyl group; alternatively, R⁴⁰, R⁴³, R⁴⁴, and R⁴⁷ can be hydrogen and R⁴¹, R⁴², R⁴⁵, and R⁴⁶ independently can be hydrogen or a C₁-C₁₀ alkyl group; or alternatively, R⁴⁰, R⁴³, R⁴⁴, and R⁴⁷ can be hydrogen and R⁴¹, R⁴², R⁴⁵, and R⁴⁶ independently can be hydrogen or a C₁-C₅ alkyl group. In any embodiment wherein R⁴¹, R⁴², R⁴⁵, and R⁴⁶ are not hydrogen, R⁴¹ and R⁴² can be joined to form a ring and/or R⁴⁵ and R⁴⁶ can be joined to form a ring. In any embodiment where R⁴¹ and R⁴² and/or are joined to form a ring, the joined group can be a C₁-C₂₀ hydrocarbylene group; alternatively, a C₁-C₁₀ hydrocarbylene group; alternatively, a C₁-C₂₀ alkylene group; a C₁-C₁₀ alkylene group; or alternatively, a C₁-C₅ alkylene group. In any embodiment, R³⁷ can be a C₁-C₁₀ hydrocarbyl group; a C₁-C₁₀ alkyl group; or alternatively, C₁-C₅ alkyl group. In any embodiment, R⁵⁰ and R⁵¹ independently can be hydrogen or a C₁-C₂₀ hydrocarbyl group; alternatively, hydrogen or a C₁-C₁₀ hydrocarbyl group; alternatively, a hydrogen or a C₁-C₂₀ alkyl group; alternatively, hydrogen or a C₁-C₁₀ alkyl group; alternatively, hydrogen or a C₁-C₅ alkyl group; alternatively, C₁-C₂₀ hydrocarbyl groups; alternatively, C₁-C₁₀ hydrocarbyl groups; alternatively, C₁-C₂₀ alkyl groups; alternatively, C₁-C₁₀ alkyl groups; or alternatively, C₁-C₅ alkyl groups. According to this aspect, constrained-geometry metallocenes are suitable for use in the catalyst system of this disclosure. In a further aspect, non-constrained geometry metallocenes also are suitable for use in the catalyst system of this disclosure. In any embodiment provided herein, X³⁰ and X³¹ independently can be a halide, a C₁ to C₂₀ hydrocarbyl group (saturated or unsaturated), a C₁ to C₂₀ hydrocarboxide group (saturated or unsaturated), a C₁ to C₂₀ aliphatic group, a C₁ to C₂₀ heterocyclic group (saturated or unsaturated), a C₆ to C₂₀ aromatic group, a C₁ to C₂₀ heteroaromatic group, a C₁-C₂₀ alkyl group, a C₁-C₂₀ alkylene group, a C₁ to C₂₀ alkoxide group, or hydrogen; alternatively, a halide; alternatively, a C₁ to C₂₀ hydrocarbyl group (saturated or unsaturated); alternatively, a C₁ to C₂₀ hydrocarboxide group (saturated or unsaturated); alternatively, a C₁ to C₂₀ aliphatic group; alternatively, a C₁ to C₂₀ heterocyclic group (saturated or unsaturated); alternatively, a C₆ to C₂₀ aromatic group; alternatively, a C₁ to C₂₀ heteroaromatic group; alternatively, a C₁-C₂₀ alkyl group; alternatively, a C₁-C₂₀ alkylene group; alternatively, a C₁ to C₂₀ alkoxide group; or alternatively, hydrogen. Also in any embodiment provided herein, X³⁰ and X³¹ independently can be a halide, a C₁ to C₁₀ hydrocarbyl group (saturated or unsaturated), a C₁ to C₁₀ hydrocarboxide group (saturated or unsaturated), a C₁ to C₁₀ aliphatic group, a C₁ to C₁₀ heterocyclic group (saturated or unsaturated), a C₆ to C₁₀ aromatic group, a C, to C₁₀ heteroaromatic group, a C₁-C₁₀ alkyl group, a C₁-C₁₀ alkylene group, a C₁ to C₁₀ alkoxide group, or hydrogen; alternatively, a halide; alternatively, a C₁ to C₁₀ hydrocarbyl group (saturated or unsaturated); alternatively, a C₁ to C₁₀ hydrocarboxide group (saturated or unsaturated); alternatively, a C₁ to C₁₀ aliphatic group; alternatively, a C₁ to C₅ heterocyclic group (saturated or unsaturated); alternatively, a C₆ to C₁₀ aromatic group; alternatively, a C₁ to C₁₀ heteroaromatic group; alternatively, a C₁-C₁₀ alkyl group; alternatively, a C₁-C₁₀ alkylene group; alternatively, a C₁ to C₁₀ alkoxide group; or alternatively, hydrogen. In still any embodiment provided herein, X³⁰ and X³¹ independently can be a halide, a C₁ to C₅ hydrocarbyl group (saturated or unsaturated), a C₁ to C₅ hydrocarboxide group (saturated or unsaturated), a C₁ to C₅ aliphatic group, a C₁ to C₅ heterocyclic group (saturated or unsaturated), a C₁-C₅ alkyl group, or a C₁ to C₅ alkoxide group; alternatively, a halide; alternatively, a C₁ to C₅ hydrocarbyl group (saturated or unsaturated); alternatively, a C₁ to C₅ hydrocarboxide group (saturated or unsaturated); alternatively, a C₁ to C₅ aliphatic group; alternatively, a C₁ to C₅ heterocyclic group (saturated or unsaturated); alternatively, a C₁-C₅ alkyl group; or alternatively, a C₁ to C5 alkoxide group.

In a non-limiting embodiment, the metallocene may have the formula

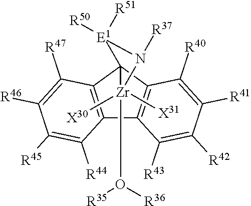

wherein, E¹, R⁴¹, R⁴², R⁴⁵, R⁴⁶, R⁵⁰ and R⁵¹, R³⁷, R³⁵, and R³⁶ can be any group and have any embodiment as provided herein. In another non-limiting embodiment, the metallocene of formula ZrR¹²R¹³R¹⁴X⁹₂ can have the formula

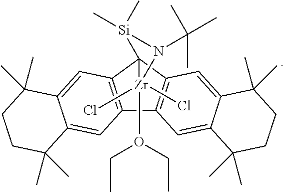

In a further aspect and in any embodiment disclosed herein, the metallocene can comprise two η⁵-cyclopentadienyl-type ligands that are connected by linking group consisting of one, two, or three bridging atoms. In a another aspect and in any embodiment disclosed herein, the metallocene can comprise one η⁵-cyclopentadienyl-type ligand that is connected by a bridge consisting of one, two, or three bridging atoms to another ligand in the metallocene that is not an η⁵-cyclopentadienyl-type ligand. Each of these bridges can be further substituted if desired. The complete, substituted bridging group or bridging atoms are described along with their substituents, other than the cyclopentadienyl-type ligand substituents, as the “linking group.” By way of example of this terminology, possible linking groups include —CH₂CH₂— or —CH(CH₃)CH(CH₃)—, both of which comprise a C₂ bridge. Thus, the —CH₂CH₂-linking group is generally described as unsubstituted linking group, while linking groups such as —CH(CH₃)CH(CH₃)— are generally described as a substituted linking group.

Additional examples of metallocenes that can be used in the various embodiments and aspects of this disclosure are provided in the following references: L. J. Irwin, J. H. Reibenspies, and S. A. Miller, J. Am. Chem. Soc. 2004, 126, 16716-16717; U.S. Pat. No. 7,214,749; WO 2006052232; WO 2008143802; WO 2009045300; WO 2009045301; WO 2007127465; and WO 2008010865; each of which are incorporated herein by reference in their entireties.

### Aluminoxanes

One aspect of this disclosure provides for a method of producing an olefin wax oligomer composition comprising contacting an olefin wax and a catalyst system, wherein the catalyst system can comprise a metallocene and an activator. In an embodiment, the activator can comprise, consist of, or consist essentially of, an aluminoxane compound. The aluminoxane compound can be used alone or in combination with any other activators disclosed herein. In an aspect of any embodiment provided here, for example, the catalyst system can comprise at least one aluminoxane as an activator, either alone or in combination with a chemically-treated solid oxide or any other activators(s). In some embodiments, the catalyst system can comprise, consist essentially of, or consist of, a metallocene and an activator comprising an aluminoxane. In other embodiments, the catalyst system can be substantially free of aluminoxanes.

Aluminoxanes are also referred to as poly(hydrocarbyl aluminum oxides), organoaluminoxanes, or alumoxanes. In a further aspect of any embodiment provided here, the catalyst system can comprise, either alone or in combination with any other activator or activators, at least one aluminoxane compound. For example, in various embodiments, the catalyst system can comprise an aluminoxane as the only activator, or can comprise an aluminoxane in combination with the chemically-treated solid oxide and/or any other activator(s). Aluminoxanes are described herein and can be utilized without limitation as used alone or in combination with any other activator or activators.

In some embodiments, the catalyst system can comprise, consist essentially of, or consist of a metallocene and an activator comprising, consisting of, or consisting essentially of an aluminoxane. In other embodiments, the catalyst system can comprise, consist essentially of, or consist of, a metallocene, a first activator comprising a chemically-treated solid oxide, and a second activator comprising an aluminoxane.

Aluminoxane compounds that can be used in the catalyst system of this disclosure include, but are not limited to, oligomeric compounds. The oligomeric aluminoxane compounds can comprise linear structures, cyclic, or cage structures, or mixtures of all three, and may further include additional structures having the general repeating formula. Oligomeric aluminoxanes, whether oligomeric or polymeric compounds, have the repeating unit formula:

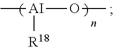

wherein
R¹⁸ is a linear or branched alkyl group. In one aspect, for example, R¹⁸ can be a linear or branched alkyl having from 1 to 10 carbon atoms, and n can be an integer from 3 to about 10, which are encompassed by this disclosure. Linear aluminoxanes having the

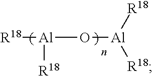

wherein
R¹⁸ can be a linear or branched alkyl group are also encompassed by this disclosure. Alkyl groups for organoaluminum compounds having the formula Al(X¹⁰)ₙ(X¹¹)₃₋ₙ have been independently described herein and these alkyl groups can be utilized, without limitation, to further describe the aluminoxanes having the structure above. Generally, n of the alumoxanes can be, or can have an average, greater than 1; or alternatively, greater than 2. In an embodiment, n can range, or have an average with the range, from 2 to 15; or alternatively, from 3 to 10. As will be appreciated by one of ordinary skill in the art, the identity and size of the R¹⁸ group and the value of n are exemplary, as a wide range of parameters and combinations thereof may occur in an aluminoxane composition and can be used.

Further, aluminoxanes can also have cage structures of the formula R¹_{5m+α}R^{b}_{m-α}Al₄ₘO₃ₘ, wherein m is 3 or 4 and α is =n_{Al(3)}−n_{O(2)}+n_{O(4)}; wherein n_{Al(3)} is the number of three coordinate aluminum atoms, n_{O(2)} is the number of two coordinate oxygen atoms, n_{O(4)} is the number of 4 coordinate oxygen atoms, R¹ represents a terminal alkyl group, and Rb represents a bridging alkyl group; wherein R is a linear or branched alkyl having from 1 to 10 carbon atoms. Alkyl groups for organoaluminum compounds having the formula Al(X¹⁰)ₙ(X¹¹)₃₋ₙ have been independently described herein and these alkyl groups can be utilized, without limitation, to further describe the aluminoxanes having the cage structure of the formula R¹_{5m+α}R^{b}_{m-α}Al₄ₘO₃ₘ.

In a non-limiting embodiment, useful aluminoxanes can include methylaluminoxane (MAO), ethylaluminoxane, modified methylaluminoxane (MMAO), n-propylaluminoxane, iso-propyl-aluminoxane, n-butylaluminoxane, sec-butylaluminoxane, iso-butylaluminoxane, t-butyl aluminoxane, 1-pentylaluminoxane, 2-pentylaluminoxane, 3-pentylaluminoxane, iso-pentylaluminoxane, neopentylaluminoxane, or mixtures thereof; In some non-limiting embodiments, useful aluminoxanes can include methylaluminoxane (MAO), modified methylaluminoxane (MMAO), isobutyl aluminoxane, t-butyl aluminoxane, or mixtures thereof. In other non-limiting embodiments, useful aluminoxanes can be methylaluminoxane (MAO); alternatively, ethylaluminoxane; alternatively, modified methylaluminoxane (MMAO); alternatively, n-propylaluminoxane; alternatively, iso-propylaluminoxane; alternatively, n-butylaluminoxane; alternatively, sec-butylaluminoxane; alternatively, iso-butylaluminoxane; alternatively, t-butyl aluminoxane; alternatively, 1-pentylaluminoxane; alternatively, 2-pentylaluminoxane; alternatively, 3-pentylaluminoxane; alternatively, iso-pentylaluminoxane; or alternatively, neopentylaluminoxane.

While organoaluminoxanes with different types of “R” groups such as R¹⁸ are encompassed by the present disclosure, methyl aluminoxane (MAO), ethyl aluminoxane, or isobutyl aluminoxane can also be utilized as aluminoxane activators used in the catalyst systems of this disclosure. These aluminoxanes are prepared from trimethylaluminum, triethylaluminum, or triisobutylaluminum, respectively, and are sometimes referred to as poly(methyl aluminum oxide), poly(ethyl aluminum oxide), and poly(isobutyl aluminum oxide), respectively. It is also within the scope of the disclosure to use an aluminoxane in combination with a trialkylaluminum, such as disclosed in U.S. Pat. No. 4,794,096, which is herein incorporated by reference in its entirety.

The present disclosure contemplates many values of n in the aluminoxane formulas [Al(R¹⁸)O]ₙ and R¹⁸ [Al(R¹⁸)O]ₙAl(R¹⁸)₂, and preferably n is at least about 3. However, depending upon how the organoaluminoxane is prepared, stored, and used, the value of n can be variable within a single sample of aluminoxane, and such a combination of organoaluminoxanes are comprised in the methods and compositions of the present disclosure.

In preparing the catalyst system that includes an aluminoxane, the molar ratio of the aluminum in the aluminoxane to the metal of the metallocene (Al:metal of the metallocene) in catalyst system can be greater than 0.1:1; alternatively, greater than 1:1; or alternatively, greater than 10:1; or alternatively, greater than 50:1. In an embodiment, the molar ratio of the aluminum in the aluminoxane to the metal of the metallocene (Al:metal of the metallocene) in catalyst system can range from 0.1:1 to 100,000:1; alternatively, range from 1:1 to 10,000:1; alternatively, range from 10:1 to 1,000:1; or alternatively, range from 50:1 to 500:1. When the metallocene contains a specific metal (e.g. Zr) the ratio can be stated as an Al:specific metal ratio (e.g Al:Zr molar ratio). In an aspect, the amount of aluminoxane added to an oligomerization zone can be in an amount within a range from 0.01 mg/L to 1000 mg/L; alternatively, from 0.1 mg/L to 100 mg/L; or alternatively, or from 1 mg/L to 50 mg/L.

Organoaluminoxanes can be prepared by various procedures which are well known in the art. Examples of organoaluminoxane preparations are disclosed in U.S. Pat. Nos. 3,242,099 and 4,808,561, each of which is incorporated by reference herein, in its entirety. One example of how an aluminoxane can be prepared is as follows. Water which is dispersed or dissolved in an inert organic solvent can be reacted with an aluminum alkyl compound such as AlR₃ to form the desired organoaluminoxane compound. While not intending to be bound by this statement, it is believed that this synthetic method can afford a mixture of both linear and cyclic [Al(R¹⁸)O]ₙ aluminoxane species, both of which are encompassed by this disclosure. Alternatively, organoaluminoxanes can be prepared by reacting an aluminum alkyl compound such as AlR₃ with a hydrated salt, such as hydrated copper sulfate, in an inert organic solvent.

The other catalyst components can be contacted with the aluminoxane in a solvent which is substantially inert to the reactants, intermediates, and products of the activation step can be used. The catalyst system formed in this manner can be collected by methods known to those of skill in the art, including but not limited to filtration, or the catalyst system can be introduced into the oligomerization reactor without being isolated.

### Chemically-Treated Solid Oxide

One aspect of this disclosure provides for an oligomerization method comprising (or a method of producing an olefin wax oligomer and/or a olefin wax oligomer composition comprising a step of) contacting an olefin wax and a catalyst system comprising a metallocene and an activator. In one aspect, this disclosure encompasses a catalyst system comprising at least one metallocene and at least one activator. One exemplary activator that can be utilized is a chemically-treated solid oxide. The term “chemically-treated solid oxide” is used interchangeably with similar terms such as, “solid oxide treated with an electron-withdrawing anion,” “treated solid oxide,” or “solid super acid,” which is also termed “SSA.” While not intending to be bound by theory, it is thought that the chemically-treated solid oxide can serve as an acidic activator-support. In one aspect and in any embodiment, the chemically-treated solid oxide can be used in combination with an organoaluminum compound or similar activating agent or alkylating agent. In one aspect and in any embodiment, the chemically-treated solid oxide can be used in combination with an organoaluminum compound. In another aspect and in any embodiment, the metallocene can be “pre-activated” by, being alkylated prior to its use in the catalyst system. In an aspect and in any embodiments, the chemically-treated solid oxide can be used as the only activator. In yet another aspect and in any embodiment, the metallocene is “pre-activated” and the chemically-treated solid oxide can be used in conjunction with another activator; or alternatively, multiple other activators.

In one aspect and any embodiment of this disclosure, the catalyst system can comprise at least one chemically-treated solid oxide comprising at least one solid oxide treated with at least one electron-withdrawing anion, wherein the solid oxide can comprise any oxide that is characterized by a high surface area, and the electron-withdrawing anion can comprise any anion that increases the acidity of the solid oxide as compared to the solid oxide that is not treated with at least one electron-withdrawing anion.

In another aspect and in any embodiment of this disclosure, the catalyst system can comprise a chemically-treated solid oxide comprising a solid oxide treated with an electron-withdrawing anion, wherein:
the solid oxide is selected from silica, alumina, silica-alumina, aluminum phosphate, heteropolytungstates, titania, zirconia, magnesia, boria, zinc oxide, mixed oxides thereof, or mixtures thereof; and
the electron-withdrawing anion is selected from fluoride, chloride, bromide, phosphate, triflate, bisulfate, sulfate, fluorophosphate, fluorosulfate, or any combination thereof.

In another aspect and in any embodiment of this disclosure, the catalyst system can comprise a chemically-treated solid oxide comprising a solid oxide treated with an electron-withdrawing anion, wherein:
the solid oxide is selected from silica, alumina, silica-alumina, titania, zirconia, mixed oxides thereof, or mixtures thereof; and
the electron-withdrawing anion is selected from fluoride, chloride, bisulfate, sulfate, or any combination thereof.

In another aspect and in any embodiment of this disclosure, the chemically-treated solid oxide can be fluorided alumina, chlorided alumina, bromided alumina, sulfated alumina, fluorided silica-alumina, chlorided silica-alumina, bromided silica-alumina, sulfated silica-alumina, fluorided silica-zirconia, chlorided silica-zirconia, bromided silica-zirconia, sulfated silica-zirconia, or any combination thereof; alternatively, fluorided alumina, chlorided alumina, sulfated alumina, fluorided silica-alumina, chlorided silica-alumina, sulfated silica-alumina, fluorided silica-zirconia, sulfated silica-zirconia, or any combination thereof; alternatively, fluorided alumina; alternatively, chlorided alumina; alternatively, bromided alumina; alternatively, sulfated alumina; alternatively, fluorided silica-alumina; alternatively, chlorided silica-alumina; alternatively, bromided silica-alumina; alternatively, sulfated silica-alumina; alternatively, fluorided silica-zirconia; alternatively, chlorided silica-zirconia; alternatively, bromided silica-zirconia; or alternatively, sulfated silica-zirconia. Further, and in yet another aspect, the chemically-treated solid oxide can further comprise a metal or metal ion selected from zinc, nickel, vanadium, silver, copper, gallium, tin, tungsten, molybdenum, or any combination thereof; alternatively, zinc, nickel, vanadium, tin, or any combination thereof; alternatively, zinc; alternatively, nickel; alternatively, vanadium; alternatively, silver; alternatively, copper; alternatively, gallium; alternatively, tin; alternatively, tungsten; or alternatively, molybdenum.

In yet a further aspect and in any embodiment of this disclosure, the chemically-treated solid oxide can comprise the contact product of at least one solid oxide compound and at least one electron-withdrawing anion source. The solid oxide compound and electron-withdrawing anion source are described independently herein and can be utilized in any combination to further describe the chemically-treated solid oxide comprising the contact product of at least one solid oxide compound and at least one electron-withdrawing anion source. That is, the chemically-treated solid oxide is provided upon contacting or treating the solid oxide with the electron-withdrawing anion source. The solid oxide compound and electron-withdrawing anion source are described independently herein and can be utilized in any combination to further describe the chemically-treated solid oxide comprising the contact product of at least one solid oxide compound and at least one electron-withdrawing anion source. In one aspect, the solid oxide compound can comprise, consist essentially of, or consist of, an inorganic oxide. It is not required that the solid oxide compound be calcined prior to contacting the electron-withdrawing anion source. The contact product can be calcined either during or after the solid oxide compound is contacted with the electron-withdrawing anion source. In this aspect, the solid oxide compound can be calcined or uncalcined; alternatively, calcined; or alternatively, uncalcined. In another aspect, the activator-support can comprise the contact product of at least one calcined solid oxide compound and at least one electron-withdrawing anion source.

While not intending to be bound by theory, the chemically-treated solid oxide, also termed the activator-support, exhibits enhanced acidity as compared to the corresponding untreated solid oxide compound. The chemically-treated solid oxide can also function as a catalyst activator as compared to the corresponding untreated solid oxide. While the chemically-treated solid oxide can activate the metallocene in the absence of additional activators, additional activators can be utilized in the catalyst system. By way of example, it may be useful to include an organoaluminum compound in the catalyst system along with the metallocene and chemically-treated solid oxide. The activation function of the activator-support is evident in the enhanced activity of catalyst system as a whole, as compared to a catalyst system containing the corresponding untreated solid oxide.

In one aspect, the chemically-treated solid oxide of this disclosure can comprise, consist essentially of, or consist of, a solid inorganic oxide material, a mixed oxide material, or a combination of inorganic oxide materials, that is chemically-treated with an electron-withdrawing component, and optionally treated with a metal; alternatively, a solid inorganic oxide material that is chemically-treated with an electron-withdrawing component and optionally treated with a metal; alternatively, a mixed oxide material that is chemically-treated with an electron-withdrawing component and optionally treated with a metal; or alternatively, a combination of inorganic oxide materials, that is chemically-treated with an electron-withdrawing component, and optionally treated with a metal. Thus, the solid oxide of this disclosure encompasses oxide materials (e.g. alumina), “mixed oxide” compounds (e.g. silica-alumina), and combinations and mixtures thereof. The mixed oxide compounds (e.g. silica-alumina) can be single or multiple chemical phases with more than one metal combined with oxygen to form a solid oxide compound, and are encompassed by this disclosure. The solid inorganic oxide material, mixed oxide material, combination of inorganic oxide materials, electron-withdrawing component, and optional metal are independently described herein and can be utilized in any combination to further described the chemically-treated solid oxide.

In one aspect of this disclosure and in any embodiment, the chemically-treated solid oxide further can comprise a metal or metal ion. In an embodiment, the metal or metal of the metal ion can comprise, consist essentially of, or consist of, zinc, nickel, vanadium, titanium, silver, copper, gallium, tin, tungsten, molybdenum, or any combination thereof; alternatively, zinc, nickel, vanadium, titanium, or tin, or any combination thereof; alternatively, zinc, nickel, vanadium, tin, or any combination thereof. In some embodiments, the metal or metal of the metal ion can comprise, consist essentially of, or consist of, zinc; alternatively, nickel; alternatively, vanadium; alternatively, titanium; alternatively, silver; alternatively, copper; alternatively, gallium; alternatively, tin; alternatively, tungsten; or alternatively, molybdenum.

In an aspect and any embodiment, the chemically-treated solid oxides that further comprise a metal or metal ion include, but are not limited to, zinc-impregnated chlorided alumina, titanium-impregnated fluorided alumina, zinc-impregnated fluorided alumina, zinc-impregnated chlorided silica-alumina, zinc-impregnated fluorided silica-alumina, zinc-impregnated sulfated alumina, chlorided zinc aluminate, fluorided zinc aluminate, sulfated zinc aluminate, or any combination thereof; alternatively, the chemically-treated solid oxide can be fluorided alumina, chlorided alumina, sulfated alumina, fluorided silica-alumina, chlorided silica-alumina, sulfated silica-alumina, fluorided silica-zirconia, sulfated silica-zirconia, or any combination thereof. In some embodiments, the chemically-treated solid oxides that further comprise a metal or metal ion can comprise, consist essentially of, or consist of, zinc-impregnated chlorided alumina; alternatively, titanium-impregnated fluorided alumina; alternatively, zinc-impregnated fluorided alumina; alternatively, zinc-impregnated chlorided silica-alumina; alternatively, zinc-impregnated fluorided silica-alumina; alternatively, zinc-impregnated sulfated alumina; alternatively, chlorided zinc aluminate; alternatively, fluorided zinc aluminate; alternatively, or sulfated zinc aluminate.

In another aspect and any embodiment, the chemically-treated solid oxide of this disclosure can comprise a solid oxide of relatively high porosity, which exhibits Lewis acidic or Brønsted acidic behavior. The solid oxide can be chemically-treated with an electron-withdrawing component, typically an electron-withdrawing anion, to form an activator-support. While not intending to be bound by the following statement, it is believed that treatment of the inorganic oxide with an electron-withdrawing component augments or enhances the acidity of the oxide. Thus in one aspect, the activator-support exhibits Lewis or Brønsted acidity which is typically greater than the Lewis or Brønsted acid strength than the untreated solid oxide, or the activator-support has a greater number of acid sites than the untreated solid oxide, or both. One method to quantify the acidity of the chemically-treated and untreated solid oxide materials is by comparing the oligomerization activities of the treated and untreated oxides under acid catalyzed reactions.

In one aspect an in any embodiment, the chemically-treated solid oxide can comprise, consist essentially of, or consist of, a solid inorganic oxide comprising oxygen and at least one element selected from Group 2, 3, 4, 5, 6, 7, 8, 9, 10, 11, 12, 13, 14, or 15 of the periodic table, or comprising oxygen and at least one element selected from the lanthanide or actinide elements; alternatively, the chemically-treated solid oxide can comprise a solid inorganic oxide comprising oxygen and at least one element selected from Group 4, 5, 6, 12, 13, or 14 of the periodic table, or comprising oxygen and at least one element selected from the lanthanide elements. (See: Hawley's Condensed Chemical Dictionary, 11^{th} Ed., John Wiley & Sons; 1995; Cotton, F. A.; Wilkinson, G.; Murillo; C. A.; and Bochmann; M. Advanced Inorganic Chemistry, 6^{th} Ed., Wiley-Interscience, 1999.) In some embodiments, the inorganic oxide can comprise oxygen and at least one element selected from Al, B, Be, Bi, Cd, Co, Cr, Cu, Fe, Ga, La, Mn, Mo, Ni, Sb, Si, Sn, Sr, Th, Ti, V, W, P, Y, Zn or Zr; alternatively, the inorganic oxide can comprise oxygen and at least one element selected from Al, B, Si, Ti, P, Zn or Zr.

In an embodiment, the solid oxide utilized in the chemically-treated solid oxide can include, but is not limited to, Al₂O₃, B₂O₃, BeO, Bi₂O₃, CdO, CO₃O₄, Cr₂O₃, CuO, Fe₂O₃, Ga₂O₃, La₂O₃, Mn₂O₃, MoO₃, NiO, P₂O₅, Sb₂O₅, SiO₂, SnO₂, SrO, ThO₂, TiO₂, V₂O₅, WO₃, Y₂O₃, ZnO, ZrO₂, mixed oxides thereof, and combinations thereof; alternatively, Al₂O₃, B₂O₃, SiO₂, SnO₂, TiO₂, V₂O₅, WO₃, Y₂O₃, ZnO, ZrO₂, including mixed oxides thereof, and combinations thereof; alternatively, Al₂O₃, SiO₂, TiO₂, ZrO₂, and the like, including mixed oxides thereof, and combinations thereof. In some embodiments, the solid oxide utilized in the chemically-treated solid oxide can comprise, consist essentially of, or consist of, Al₂O₃; alternatively, B₂O₃; alternatively, BeO; alternatively, Bi₂O₃; alternatively, CdO; alternatively, CO₃O₄; alternatively, Cr₂O₃; alternatively, CuO; alternatively, Fe₂O₃; alternatively, Ga₂O₃; alternatively, La₂O₃; alternatively, Mn₂O₃; alternatively, MoO₃; alternatively, NiO; alternatively, P₂O₅; alternatively, Sb₂O₅; alternatively, SiO₂; alternatively, SnO₂; alternatively, SrO; alternatively, ThO₂; alternatively, TiO₂; alternatively, V₂O₅; alternatively, WO₃; alternatively, Y₂O₃; alternatively, ZnO; or alternatively, ZrO₂. In an embodiment, the mixed oxides that can be used in the activator-support of the present disclosure include, but are not limited to, silica-alumina, silica-titania, silica-zirconia, zeolites, clay minerals, alumina-titania, alumina-zirconia, and zinc-aluminate; alternatively, silica-alumina, silica-titania, silica-zirconia, alumina-titania, alumina-zirconia, and zinc-aluminate; alternatively, silica-alumina, silica-titania, silica-zirconia, and alumina-titania. In some embodiments, the mixed oxides that can be used in the activator-support of the present disclosure can comprise, consist essentially of, or consist of, silica-alumina; alternatively, silica-titania; alternatively, silica-zirconia; alternatively, zeolites; alternatively, clay minerals; alternatively, alumina-titania; alternatively, alumina-zirconia; alternatively, and zinc-aluminate. In some embodiments, aluminosilicates such as clay minerals, calcium aluminosilicate, or sodium aluminosilicate are useful oxides that can be used in the activator-support of the present disclosure.

In one aspect and any embodiment of this disclosure, the solid oxide material is chemically-treated by contacting it with at least one electron-withdrawing component (e.g. an electron-withdrawing anion source). Further, the solid oxide material can be chemically-treated with a metal ion if desired, then calcined to form a metal-containing or metal-impregnated chemically-treated solid oxide. Alternatively, a solid oxide material and an electron-withdrawing anion source can be contacted and calcined simultaneously. The method by which the oxide is contacted with an electron-withdrawing component (e.g. a salt or an acid of an electron-withdrawing anion), includes, but is not limited to, gelling, co-gelling, impregnation of one compound onto another, and the like. Typically, following any contacting method, the contacted mixture of oxide compound, electron-withdrawing anion, and the metal ion, if present, can be calcined.

The electron-withdrawing component used to treat the oxide can be any component that increases the Lewis or Brønsted acidity of the solid oxide upon treatment. In one aspect, the electron-withdrawing component can be an electron-withdrawing anion derived from a salt, an acid, or other compound (e.g. a volatile organic compound) that can serve as a source or precursor for that anion. In an aspect, electron-withdrawing anions include, but are not limited to, sulfate, bisulfate, fluoride, chloride, bromide, iodide, fluorosulfate, fluoroborate, phosphate, fluorophosphate, trifluoroacetate, triflate, fluorozirconate, fluorotitanate, trifluoroacetate, triflate, and combinations thereof; alternatively, sulfate, bisulfate, fluoride, chloride, fluorosulfate, fluoroborate, phosphate, fluorophosphate, trifluoroacetate, triflate, fluorozirconate, fluorotitanate, and combinations thereof; alternatively, fluoride, chloride, bisulfate, sulfate, combinations thereof; alternatively, sulfate, bisulfate, and combinations thereof; alternatively, fluoride, chloride, bromide, iodide, and combinations thereof; alternatively, fluorosulfate, fluoroborate, trifluoroacetate, triflate, fluorozirconate, fluorotitanate, trifluoroacetate, triflate, and combinations thereof; alternatively, fluoride, chloride, combinations thereof; or alternatively, bisulfate, sulfate, combinations thereof. In some embodiments, the electron-withdrawing anion can comprise, consist essentially of, or consist of, sulfate; alternatively, bisulfate; alternatively, fluoride; alternatively, chloride; alternatively, bromide; alternatively, iodide; alternatively, fluorosulfate; alternatively, fluoroborate; alternatively, phosphate; alternatively, fluorophosphate; alternatively, trifluoroacetate; alternatively, triflate; alternatively, fluorozirconate; alternatively, fluorotitanate; alternatively, trifluoroacetate; or alternatively, triflate. In addition, other ionic or non-ionic compounds that serve as sources for these electron-withdrawing anions can also be employed in the present disclosure.

When the electron-withdrawing component comprises a salt of an electron-withdrawing anion, the counterion or cation of that salt can be any cation that allows the salt to revert or decompose back to the acid during calcining. Factors that dictate the suitability of the particular salt to serve as a source for the electron-withdrawing anion include, but are not limited to, the solubility of the salt in the desired solvent, the lack of adverse reactivity of the cation, ion-pairing effects between the cation and anion, hygroscopic properties imparted to the salt by the cation and thermal stability of the anion. In an aspect, suitable cations in the salt of the electron-withdrawing anion include, but are not limited to, ammonium, trialkyl ammonium, tetraalkyl ammonium, tetraalkyl phosphonium, H⁺, and [H(OEt₂)₂]⁺; alternatively, ammonium; alternatively, trialkyl ammonium; alternatively, tetraalkyl ammonium; alternatively, tetraalkyl phosphonium; alternatively, H⁺; or alternatively, [H(OEt₂)₂]⁺. Alkyl groups have been described herein and may be utilized without limitation at the alkyl groups of the trialkyl ammonium, tetraalkyl ammonium and tetraalkyl phosphonium compounds.

Further, combinations of one or more different electron withdrawing anions, in varying proportions, can be used to tailor the specific acidity of the activator-support to the desired level. Combinations of electron withdrawing components can be contacted with the oxide material simultaneously or individually, and any order that affords the desired chemically-treated solid oxide acidity. For example, one aspect of this disclosure is employing two or more electron-withdrawing anion source compounds in two or more separate contacting steps. In an non-limiting aspect of such a process by which an chemically-treated solid oxide is prepared can be as follows: a selected solid oxide compound, or combination of oxide compounds, is contacted with a first electron-withdrawing anion source compound to form a first mixture, this first mixture is then calcined, the calcined first mixture is then contacted with a second electron-withdrawing anion source compound to form a second mixture, followed by calcining said second mixture to form a treated solid oxide compound. In such a process, the first and second electron-withdrawing anion source compounds are typically different compounds, although they can be the same compound.

In one aspect of the disclosure, the solid oxide activator-support (chemically-treated solid oxide) can be produced by a process comprising:
1) contacting a solid oxide compound with at least one electron-withdrawing anion source compound to form a first mixture; and
2) calcining the first mixture to form the solid oxide activator-support.

In another aspect of this disclosure, the solid oxide activator-support (chemically-treated solid oxide) can be produced by a process comprising:
1) contacting at least one solid oxide compound with a first electron-withdrawing anion source compound to form a first mixture;
2) calcining the first mixture to produce a calcined first mixture;
3) contacting the calcined first mixture with a second electron-withdrawing anion source compound to form a second mixture; and
4) calcining the second mixture to form the solid oxide activator-support.
   The solid oxide activator-support may be sometimes referred to simply as a treated solid oxide compound.

In another aspect of this disclosure, the chemically-treated solid oxide can be produced or formed by contacting at least one solid oxide with at least one electron-withdrawing anion source compound, wherein the at least one solid oxide compound is calcined before, during, or after contacting the electron-withdrawing anion source, and wherein there is a substantial absence of aluminoxanes and organoborates. In an embodiment, the chemically-treated solid oxide can be produced or formed by contacting at least one solid oxide with at least one electron-withdrawing anion source compound, wherein the at least one solid oxide compound is calcined before contacting the electron-withdrawing anion source, and wherein there is a substantial absence of aluminoxanes and organoborates; alternatively, by contacting at least one solid oxide with at least one electron-withdrawing anion source compound, wherein the at least one solid oxide compound is calcined during contacting the electron-withdrawing anion source, and wherein there is a substantial absence of aluminoxanes and organoborates; or alternatively, by contacting at least one solid oxide with at least one electron-withdrawing anion source compound, wherein the at least one solid oxide compound is calcined after contacting the electron-withdrawing anion source, and wherein there is a substantial absence of aluminoxanes and organoborates.

In one aspect of this disclosure, once the solid oxide has been treated and dried, it can be subsequently calcined. Calcining of the treated solid oxide is generally conducted in an ambient atmosphere; alternatively, in a dry ambient atmosphere. The solid oxide can be calcined at a temperature from 200° C. to 900° C.; alternatively, from 300° C. to 800° C.; alternatively, from 400° C. to 700° C.; or alternatively, from 350° C. to 550° C. The period of time at which the solid oxide is maintained at the calcining temperature can be 1 minute to 100 hours; alternatively, from 1 hour to 50 hours; alternatively, from 3 hours to 20 hours; or alternatively, from 1 to 10 hours.

Further, any type of suitable atmosphere can be used during calcining. Generally, calcining is conducted in an oxidizing atmosphere, such as air. Alternatively, an inert atmosphere, such as nitrogen or argon, or a reducing atmosphere such as hydrogen or carbon monoxide, can be used. In an embodiment, the atmosphere utilized for calcining can comprise, or consist essentially of air, nitrogen, argon, hydrogen, or carbon monoxide, or any combination thereof; alternatively, nitrogen, argon, hydrogen, carbon monoxide, or any combination thereof; alternatively, air; alternatively, nitrogen; alternatively, argon; alternatively, hydrogen; or alternatively, carbon monoxide.

In another aspect and any embodiment of the disclosure, the solid oxide component used to prepare the chemically-treated solid oxide can have a pore volume greater than 0.1 cc/g. In another aspect, the solid oxide component can have a pore volume greater than 0.5 cc/g; alternatively, greater than 1.0 cc/g. In still another aspect, the solid oxide component can have a surface area from 100 to 1000 m²/g. In another aspect, solid oxide component can have a surface area from 200 to 800 m²/g; alternatively, from 250 to 600 m²/g.

The solid oxide material can be treated with a source of halide ion, sulfate ion, or a combination thereof, and optionally treated with a metal ion, then calcined to provide the chemically-treated solid oxide in the form of a particulate solid. In one aspect, the solid oxide material is treated with a source of sulfate (termed a sulfating agent), a source of phosphate (termed a phosphating agent), a source of iodide ion (termed a iodiding agent), a source of bromide ion (termed a bromiding agent), a source of chloride ion (termed a chloriding agent), a source of fluoride ion (termed a fluoriding agent), or any combination thereof, and calcined to provide the solid oxide activator. In another aspect, useful acidic activator-supports can comprise, consist essentially of, or consist of, iodided alumina, bromided alumina, chlorided alumina, fluorided alumina, sulfated alumina, phosphated alumina, iodided silica-alumina, bromided silica-alumina, chlorided silica-alumina, fluorided silica-alumina, sulfated silica-alumina, phosphated silica-alumina, iodided silica-zirconia, bromided silica-zirconia, chlorided silica-zirconia, fluorided silica-zirconia, sulfated silica-zirconia, phosphated silica-zirconia, a pillared clay (e.g. a pillared montmorillonite) treated with iodide, bromide, chloride, fluoride, sulfate, or phosphate, an aluminophosphate (e.g. a molecular sieve) treated with iodide, bromide, chloride, fluoride, sulfate, or phosphate, or any combination of these acidic activator-supports. Further, any of the activator-supports can optionally be treated with a metal ion, as provided herein.

Alternatively, useful acidic activator-supports can comprise, consist essentially of, or consist of, chlorided alumina, fluorided alumina, sulfated alumina, phosphated alumina, chlorided silica-alumina, fluorided silica-alumina, sulfated silica-alumina, chlorided silica-zirconia, fluorided silica-zirconia, sulfated silica-zirconia, an aluminophosphate treated with sulfate, fluoride, or chloride, or any combination of these acidic activator-supports. Moreover, the solid oxide can be treated with more than one electron-withdrawing anion, for example, the acidic activator-support can be or can comprise, consist essentially of, or consist of, an aluminophosphate or aluminosilicate treated with sulfate and fluoride, silica-alumina treated with fluoride and chloride; or alumina treated with phosphate and fluoride.

Alternatively and in another aspect, useful acidic activator-supports can comprise, consist essentially of, or consist of, fluorided alumina, sulfated alumina, fluorided silica-alumina, sulfated silica-alumina, fluorided silica-zirconia, sulfated silica-zirconia, or phosphated alumina, or any combination of these acidic activator-supports. In yet another aspect, useful acidic activator-supports can comprise, consist essentially of, or consist of, iodided alumina; alternatively, bromided alumina; alternatively, chlorided alumina; alternatively, fluorided alumina; alternatively, sulfated alumina; alternatively, phosphated alumina; alternatively, iodided silica-alumina; alternatively, bromided silica-alumina; alternatively, chlorided silica-alumina; alternatively, fluorided silica-alumina; alternatively, sulfated silica-alumina; alternatively, phosphated silica-alumina; alternatively, iodided silica-zirconia; alternatively, bromided silica-zirconia; alternatively, chlorided silica-zirconia; alternatively, fluorided silica-zirconia; alternatively, sulfated silica-zirconia; alternatively, phosphated silica-zirconia; alternatively, a pillared clay (e.g. a pillared montmorillonite); alternatively, an iodided pillared clay; alternatively, a bromided pillared clay; alternatively, a chlorided pillared clay; alternatively, a fluorided pillared clay; alternatively, a sulfated pillared clay; alternatively, a phosphated pillared clay; alternatively, an iodided aluminophosphate; alternatively, a bromided aluminophosphate; alternatively, a chlorided aluminophosphate; alternatively, a fluorided aluminophosphate; alternatively, a sulfated aluminophosphate; alternatively, a phosphated aluminophosphate; or any combination of these acidic activator-supports. Again, any of the activator-supports disclosed herein can optionally be treated with a metal ion.

In one aspect of this disclosure, the chemically-treated solid oxide can comprise, consist essentially of, or consist of, a fluorided solid oxide in the form of a particulate solid, where a source of fluoride ion is added to the solid oxide by treatment with a fluoriding agent. In still another aspect, fluoride ion can be added to the solid oxide by forming a slurry of the solid oxide in a suitable solvent. In an embodiment, the solvent can be alcohol, water, or a combination thereof; alternatively, alcohol; or alternatively, water. In an embodiment suitable alcohols can have from one to three carbon alcohols because of their volatility and low surface tension. In another aspect of the present disclosure, the solid oxide can be treated with a fluoriding agent during the calcining step. Any fluoriding agent capable of serving as a source of fluoride and thoroughly contacting the solid oxide during the calcining step can be used. In an non-limiting embodiment, fluoriding agents that can be used in this disclosure include, but are not limited to, hydrofluoric acid (HF), ammonium fluoride (NH₄F), ammonium bifluoride (NH₄HF₂), ammonium tetrafluoroborate (NH₄BF₄), ammonium silicofluoride (hexafluorosilicate) ((NH₄)₂SiF₆), ammonium hexafluorophosphate (NH₄ PF₆), and combinations thereof; alternatively, hydrofluoric acid (HF), ammonium fluoride (NH₄F), ammonium bifluoride (NH₄HF₂), ammonium tetrafluoroborate (NH₄BF₄), and combinations thereof. In other non-limiting embodiments, the fluoriding agents can comprise, consist essentially of, or consist of hydrofluoric acid (HF); alternatively, ammonium fluoride (NH₄F); alternatively, ammonium bifluoride (NH₄HF₂); alternatively, ammonium tetrafluoroborate (NH₄BF₄); alternatively, ammonium silicofluoride (hexafluorosilicate) ((NH₄)₂SiF₆); or alternatively, ammonium hexafluorophosphate (NH₄ PF₆). For example, ammonium bifluoride NH₄HF₂ can be used as the fluoriding agent, due to its ease of use and ready availability.

In another aspect of the present disclosure, the solid oxide can be treated with a fluoriding agent during the calcining step. Any fluoriding agent capable of thoroughly contacting the solid oxide during the calcining step can be used. For example, in addition to those fluoriding agents described previously, volatile organic fluoriding agents can be used. Volatile organic fluoriding agents useful in this aspect of the disclosure include, but are not limited to, freons, perfluorohexane, perfluorobenzene, fluoromethane, trifluoroethanol, and combinations thereof. In some embodiments, the volatile fluoriding agent can comprise, consist essentially of, or consist of, a freon; alternatively, perfluorohexane; alternatively, perfluorobenzene; alternatively, fluoromethane; or alternatively, trifluoroethanol. Gaseous hydrogen fluoride or fluorine itself can also be used with the solid oxide is fluorided during calcining. One convenient method of contacting the solid oxide with the fluoriding agent is to vaporize a fluoriding agent into a gas stream used to fluidize the solid oxide during calcination.

Similarly, in another aspect of this disclosure, the chemically-treated solid oxide can comprise, consist essentially of, or consist of, a chlorided solid oxide in the form of a particulate solid, where a source of chloride ion is added to the solid oxide by treatment with a chloriding agent. The chloride ion can be added to the solid oxide by forming a slurry of the solid oxide in a suitable solvent. In an embodiment, the solvent can be alcohol, water, or a combination thereof; alternatively, alcohol; or alternatively, water. In an embodiment suitable alcohols can have from one to three carbon alcohols because of their volatility and low surface tension. In another aspect of the present disclosure, the solid oxide can be treated with a chloriding agent during the calcining step. Any chloriding agent capable of serving as a source of chloride and thoroughly contacting the solid oxide during the calcining step can be used. In a non-limiting embodiment, volatile organic chloriding agents can be used. In some embodiments, the volatile organic chloriding agents include, but are not limited to, chloride containing freons, perchlorobenzene, chloromethane, dichloromethane, trichloroethane, tetrachloroethylene, chloroform, carbon tetrachloride, trichloroethanol, or any combination thereof. In some embodiments, the volatile organic chloriding agents can comprise, consist essentially of, or consist of, chloride containing freons; alternatively, perchlorobenzene; alternatively, chloromethane; alternatively, dichloromethane; alternatively, chloroform; alternatively, carbon tetrachloride; or alternatively, trichloroethanol. Gaseous hydrogen chloride or chlorine itself can also be used with the solid oxide during calcining. One convenient method of contacting the oxide with the chloriding agent is to vaporize a chloriding agent into a gas stream used to fluidize the solid oxide during calcination.

In still another aspect, the chemically-treated solid oxide can comprise, consist essentially of, or consist of, a bromided solid oxide in the form of a particulate solid, where a source of bromide ion is added to the solid oxide by treatment with a bromiding agent. The bromide ion can be added to the solid oxide by forming a slurry of the solid oxide in a suitable solvent. In an embodiment, the bromiding solvent can be alcohol, water, or a combination thereof; alternatively, alcohol; or alternatively, water. In an embodiment suitable alcohols can have from one to three carbon alcohols because of their volatility and low surface tension. In another aspect of the present disclosure, the solid oxide can be treated with a bromiding agent during the calcining step. Any bromiding agent capable of serving as a source of bromide and thoroughly contacting the solid oxide during the calcining step can be used. In a non-limiting embodiment, volatile organic bromiding agents can be used. In some embodiments, the volatile organic chloriding agents include, but are not limited to, bromide containing freons, bromomethane, dibromomethane, tribromoethane, tetrabromoethylene, bromoform, carbon tetrabromide, tribromoethanol, or any combination thereof. In some embodiments, the volatile organic chloriding agents can comprise, consist essentially of, or consist of, bromide containing freons; alternatively, bromomethane; alternatively, dibromomethane; alternatively, bromoform; alternatively, carbon tetrabromide; or alternatively, tribromoethanol. Gaseous hydrogen bromide or bromine itself can also be used with the solid oxide during calcining. One convenient method of contacting the oxide with the bromiding agent is to vaporize a bromiding agent into a gas stream used to fluidize the solid oxide during calcination.

In one aspect, the amount of fluoride ion, chloride ion, or bromide ion present before calcining the solid oxide is generally from 2% to 50% by weight, where the weight percents are based on the weight of the solid oxide, before calcining. In another aspect, the amount of fluoride or chloride ion present before calcining the solid oxide is from 3% to 25% by weight; alternatively, from 4% to 20% by weight. Once impregnated with halide, the halided solid oxide can be dried by any method known in the art including, but not limited to, suction filtration followed by evaporation, drying under vacuum, spray drying, and the like. In an embodiment, the calcining step can be initialed without drying the impregnated solid oxide.

In an aspect, silica-alumina, or a combination thereof can be utilized as the solid oxide material. The silica-alumina used to prepare the treated silica-alumina can have a pore volume greater than 0.5 cc/g. In one aspect, the pore volume can be greater than 0.8 cc/g; alternatively, greater than 1 cc/g. Further, the silica-alumina can have a surface area greater than 100 m²/g. In one aspect, the surface area is greater than 250 m²/g; alternatively, greater than 350 m²/g. Generally, the silica-alumina has an alumina content from 5% to 95%. In one aspect, the alumina content of the silica-alumina can be from 5 to 50%; alternatively, from 8% to 30% alumina by weight. In yet other aspects, the solid oxide component can comprise alumina without silica, or silica without alumina.

In another aspect, the chemically-treated solid oxide can comprise, consist essentially of, or consist of, a sulfated solid oxide in the form of a particulate solid, where a source of sulfate ion is added to the solid oxide by treatment with a sulfating agent. The sulfated solid oxide can comprise sulfate and a solid oxide component any solid oxide component described (e.g. alumina or silica-alumina), in the form of a particulate solid. The sulfated solid oxide can be further treated with a metal ion if desired such that the calcined sulfated solid oxide can comprise a metal. In one aspect, the sulfated solid oxide can comprise sulfate and alumina; alternatively, the sulfated solid oxide can comprise sulfate and silica-alumina. In one aspect of this disclosure, the sulfated alumina is formed by a process wherein the alumina or silica alumina is treated with a sulfate source. Any sulfate source capable of thoroughly contacting the solid oxide can be utilized. In an embodiment, the sulfate source may include, but is not limited to, sulfuric acid or a sulfate containing salt (e.g. ammonium sulfate). In one aspect, this process can be performed by forming a slurry of the solid oxide in a suitable solvent. In an embodiment, the solvent can be alcohol, water, or a combination thereof; alternatively, alcohol; or alternatively, water. In an embodiment suitable alcohols can have from one to three carbon alcohols because of their volatility and low surface tension.

In one aspect and any embodiment of the disclosure, the amount of sulfate ion present before calcining is generally from 0.5 parts by weight to 100 parts by weight sulfate ion to 100 parts by weight solid oxide. In another aspect, the amount of sulfate ion present before calcining is generally from 1 part by weight to 50 parts by weight sulfate ion to 100 parts by weight solid oxide; alternatively, from 5 parts by weight to 30 parts by weight sulfate ion to 100 parts by weight solid oxide. Once impregnated with sulfate, the sulfated solid oxide can be dried by any method known in the art including, but not limited to, suction filtration followed by evaporation, drying under vacuum, spray drying, and the like. In an embodiment, the calcining step can be initiated without drying the impregnated solid oxide.

In still another aspect, the chemically-treated solid oxide can comprise, consist essentially of, or consist of, a phosphated solid oxide in the form of a particulate solid, where a source of phosphate ion is added to the solid oxide by treatment with a phosphating agent. The phosphated solid oxide can comprise phosphate and any solid oxide component described (e.g. alumina or silica-alumina), in the form of a particulate solid. The phosphated solid oxide can be further treated with a metal ion if desired such that the calcined phosphated solid oxide can comprise a metal. In one aspect, the phosphated solid oxide can comprise phosphate and alumina; alternatively phosphate and silica-alumina. In one aspect of this disclosure, the phosphated alumina is formed by a process wherein the alumina or silica-alumina is treated with a phosphate source. Any phosphate source capable of thoroughly contacting the solid oxide can be utilized. In an embodiment, the phosphate source can include, but is not limited to, phosphoric acid, phosphorous acid, or a phosphate containing salt (e.g. ammonium phosphate). In one aspect, this process can be performed by forming a slurry of the solid oxide in a suitable solvent. In an embodiment, the solvent can be alcohol, water, or combination thereof; alternatively, alcohol; or alternatively, water. In an embodiment suitable alcohols can have from one to three carbon alcohols because of their volatility and low surface tension.

In one aspect and any embodiment of the disclosure, the amount of phosphate ion present before calcining is generally from 0.5 parts by weight to 100 parts by weight phosphate ion to 100 parts by weight solid oxide. In another aspect, the amount of phosphate ion present before calcining is generally from 1 part by weight to 50 parts by weight phosphate ion to 100 parts by weight solid oxide; alternatively, from 5 parts by weight to 30 parts by weight phosphate ion to 100 parts by weight solid oxide. Once impregnated with sulfate, the phosphate solid oxide can be dried by any method known in the art including, but not limited to, suction filtration followed by evaporation, drying under vacuum, spray drying, and the like. In an embodiment, the calcining step can be initiated without drying the impregnated solid oxide.

In addition to being treated with an electron-withdrawing component (for example, halide or sulfate ion), the solid inorganic oxide of this disclosure can be optionally treated with a metal source. In an embodiment, the metal source can be a metal salt or a metal-containing compound. In one aspect of the disclosure, the metal salt of metal containing compound can be added to or impregnated onto the solid oxide in solution form and converted into the supported metal upon calcining. Accordingly, the metal impregnated onto the solid inorganic oxide can comprise, consist essentially of, or consist of, zinc, titanium, nickel, vanadium, silver, copper, gallium, tin, tungsten, molybdenum, or a combination thereof; alternatively, zinc, titanium, nickel, vanadium, silver, copper, tin, or any combination thereof; alternatively, zinc, nickel, vanadium, tin, or any combination thereof. In an embodiment, the metal impregnated onto the solid inorganic oxide can comprise, consist essentially of, or consist of, zinc; alternatively, titanium; alternatively, nickel; alternatively, vanadium; alternatively, silver; alternatively, copper; alternatively, gallium; alternatively, tin; alternatively, tungsten; or alternatively, molybdenum. In some embodiments, zinc can be used to impregnate the solid oxide because it provides good catalyst activity and low cost. The solid oxide can be treated with metal salts or metal-containing compounds before, after, or at the same time that the solid oxide is treated with the electron-withdrawing anion; alternatively, before the solid oxide is treated with the electron-withdrawing anion; alternatively, after the solid oxide is treated with the electron-withdrawing anion; or alternatively, at the same time that the solid oxide is treated with the electron-withdrawing anion.

Further, any method of impregnating the solid oxide material with a metal can be used. The method by which the solid oxide is contacted with a metal source (e.g. a metal salt or metal-containing compound), includes, but is not limited to, gelling, co-gelling, and impregnation of one compound onto another. Following any contacting method, the contacted mixture of solid oxide, electron-withdrawing anion, and the metal ion is typically calcined. Alternatively, a solid oxide, an electron-withdrawing anion source, and the metal salt or metal-containing compound are contacted and calcined simultaneously.

In an aspect, the metallocene or combination of metallocenes can be precontacted with an olefin wax monomer and/or an organoaluminum compound for a first period of time prior to contacting this mixture with the chemically-treated solid oxide. Once the precontacted mixture of the metallocene, olefin wax monomer, and/or organoaluminum compound is contacted with the chemically-treated solid oxide, the composition further comprising the chemically-treated solid oxide is termed the “postcontacted” mixture. The postcontacted mixture can be allowed to remain in further contact for a second period of time prior to being charged into the reactor in which the oligomerization process will be carried out.

Various chemically-treated solid oxides and various processes to prepare chemically-treated solid oxides that can be employed in this disclosure have been reported. The following U.S. patents and published U.S. patent application provide such disclosure, and each of these patents and publications is incorporated by reference herein in its entirety: U.S. Pat. Nos. 6,107,230, 6,165,929, 6,294,494, 6,300,271, 6,316,553, 6,355,594, 6,376,415, 6,391,816, 6,395,666, 6,524,987, 6,548,441, 6,750,302, 6,831,141, 6,936,667, 6,992,032, 7,601,665, 7,026,494, 7,148,298, 7,470,758, 7,517,939, 7,576,163, 7,294,599, 7,629,284, 7,501,372, 7,041,617, 7,226,886, 7,199,073, 7,312,283, 7,619,047, and 2010/0076167, among other patents.

### The Organoaluminum Compound

One aspect of this disclosure provides for a method of producing an olefin wax oligomer and/or an olefin wax oligomer composition comprising contacting an olefin wax and a catalyst system, wherein the catalyst system can comprise a metallocene and an activator. In an embodiment, the activator can comprise, consist of, or consist essentially of an organoaluminum compound. The organoaluminum compound can be used alone or in combination with any other activators disclosed herein. In an aspect of any embodiment provided here, for example, the catalyst system can comprise at least one organoaluminum compound as an activator, either alone or in combination with a chemically-treated solid oxide, an aluminoxane, or any other activators(s). In some embodiments, the catalyst system can comprise, consist essentially of, or consist of a metallocene, a first activator comprising a chemically-treated solid oxide, and a second activator comprising an organoaluminum compound.

In an aspect, organoaluminum compounds that can be used in the catalyst system of this disclosure include but are not limited to compounds having the formula:

Al(X¹⁰)ₙ(X¹¹)₃₋ₙ.

In an embodiment, each X¹⁰ can be independently a C₁ to C₂₀ hydrocarbyl group; alternatively, a C₁ to C₁₀ hydrocarbyl group; alternately, a C₆ to C₂₀ aryl group; alternatively, a C₆ to C₁₀ aryl group; alternatively, a C₁ to C₂₀ alkyl group; alternatively, a C₁ to C₁₀ alkyl group; or alternatively, a C₁ to C₅ alkyl group. In an embodiment, each X¹¹ can be independently a halide, a hydride, or a C₁ to C₂₀ hydrocarboxide group (also referred to as a hydrocarboxy group); alternatively, a halide, a hydride, or a Cᵢ to C₁₀ hydrocarboxide group; alternatively, a halide, a hydride, or a C₆ to C₂₀ aryloxide group (also referred to as an aroxide or aroxy group); alternatively, a halide, a hydride, or a C₆ to C₁₀ aryloxide group; alternatively, a halide, a hydride, or a C₁ to C₂₀ alkoxide group (also referred to as an alkoxy group); alternatively, a halide, a hydride, or a C₁ to C₁₀ alkoxide group; alternatively, a halide, a hydride, or, or a C₁ to C₅ alkoxide group; alternatively, a halide; alternatively, a hydride; alternatively, a C₁ to C₂₀ hydrocarboxide group; alternatively, a C₁ to C₁₀ hydrocarboxide group; alternatively, a C₆ to C₂₀ aryloxide group; alternatively, a C₆ to C₁₀ aryloxide group; alternatively, a C₁ to C₂₀ alkoxide group; alternatively, a C₁ to C₁₀ alkoxide group; alternatively, a C₁ to C₅ alkoxide group. In an embodiment, n can be a number (whole or otherwise) from 1 to 3, inclusive; alternatively, about 1.5, alternatively, or alternatively, 3.

In an embodiment, each alkyl group(s) of the organoaluminum compound having the formula Al(X¹⁰)ₙ(X¹¹)₃₋ₙ can be independently a methyl group, an ethyl group, a propyl group, a butyl group, a pentyl group, a hexyl group, a heptyl group, or an octyl group; alternatively, a methyl group, a ethyl group, a butyl group, a hexyl group, or an octyl group. In some embodiments, each alkyl group(s) of the organoaluminum compound having the formula Al(X¹⁰)ₙ(X¹¹)₃₋ₙ can be independently a methyl group, an ethyl group, an n-propyl group, an n-butyl group, an iso-butyl group, a n-hexyl group, or an n-octyl group; alternatively, a methyl group, an ethyl group, a n-butyl group, or an iso-butyl group; alternatively, a methyl group; alternatively, an ethyl group; alternatively, an n-propyl group; alternatively, an n-butyl group; alternatively, an iso-butyl group; alternatively, a n-hexyl group; or alternatively, an n-octyl group. In an embodiment, each aryl group of the organoaluminum compound having the formula Al(X¹⁰)ₙ(X¹¹)₃₋ₙ can be independently a phenyl group or a substituted phenyl group; alternatively, a phenyl group; or alternatively, a substituted phenyl group. Substituted phenyl groups are described herein and these substituted phenyl group may be utilized without limitation for the organoaluminum compound having the formula Al(X¹⁰)ₙ(X¹¹)₃₋ₙ.

In an embodiment, each halide of the organoaluminum compound having the formula Al(X¹⁰)ₙ(X¹¹)₃₋ₙ can be independently a fluoride, chloride, bromide, or iodide. In some embodiments, each halide of the organoaluminum compound having the formula Al(X¹⁰)ₙ(X¹¹)₃₋ₙ can be independently a fluoride; alternatively, chloride; alternatively, bromide; or alternatively, iodide.

In an embodiment, each alkoxide of the organoaluminum compound having the formula) Al(X¹⁰)ₙ(X¹¹)₃₋ₙ can be independently a methoxy group, an ethoxy group, a propoxy group, a butoxy group, a pentoxy group, a hexoxy group, a heptoxy group, or an octoxy group; alternatively, a methoxy group, a ethoxy group, a butoxy group, a hexoxy group, or an octoxy group. In some embodiments, the alkoxy group can be independently a methoxy group, an ethoxy group, an n-propoxy group, an n-butoxy group, an iso-butoxy group, a n-hexoxy group, or an n-octoxy group; alternatively, a methoxy group, an ethoxy group, a n-butoxy group, or an iso-butoxy group; alternatively, a methoxy group; alternatively, an ethoxy group; alternatively, an n-propoxy group; alternatively, an n-butoxy group; alternatively, an iso-butoxy group; alternatively, a n-hexoxy group; or alternatively, an n-octoxy group. In an embodiment, each aryloxide of the organoaluminum compound having the formula Al(X¹⁰)ₙ(X¹¹)₃₋ₙ can be independently a be a phenoxide or a substituted phenoxide; alternatively, a phenoxide; or alternatively, a substituted phenoxide.

In an embodiment, the organoaluminum compound that can utilized in any aspect or embodiment of this disclosure can comprise, consist essentially of, or consist of, a trialkylaluminum, a dialkylaluminium halide, an alkylaluminum dihalide, a dialkylaluminum alkoxide, an alkylaluminum dialkoxide, a dialkylaluminum hydride, an alkylaluminum dihydride, or any combination thereof. In other embodiments, the organoaluminum compound that can utilized in any aspect or embodiment of this disclosure can comprise, consist essentially of, or consist of, a trialkylaluminum, a dialkylaluminium halide, an alkylaluminum dihalide, or any combination thereof; alternatively, a trialkylaluminum; alternatively, a dialkylaluminium halide; alternatively, an alkylaluminum dihalide; alternatively, a dialkylaluminum alkoxide; alternatively, an alkylaluminum dialkoxide; alternatively, a dialkylaluminum hydride; or alternatively, an alkylaluminum dihydride. In yet other embodiments, the organoaluminum compound that that can utilized in any aspect or embodiment of this disclosure can comprise, consist essentially of, or consist of, a trialkylaluminum, an alkylaluminum halide, or any combination thereof; alternatively, a trialkylaluminum; or alternatively, an alkylaluminum halide.

In a non-limiting embodiment, useful trialkylaluminum compounds can include trimethylaluminum, triethylaluminum, tripropylaluminum, tributylaluminum, trihexylalumirium, trioctylaluminum, or mixtures thereof. In some non-limiting embodiments, useful trialkylaluminum compounds can include trimethylaluminum, triethylaluminum, tripropylaluminum, tri-n-butylaluminum, tri-isobutylaluminum, trihexylaluminum, tri-n-octylaluminum, or mixtures thereof; alternatively, triethylaluminum, tri-n-butylaluminum, tri-isobutylaluminum, tri-n-hexylaluminum, tri-n-octylaluminum, or mixtures thereof; alternatively, triethylaluminum, tri-n-butylaluminum, tri-n-hexylaluminum, tri-n-octylaluminum, or mixtures thereof. In other non-limiting embodiments, useful trialkylaluminum compounds can be trimethylaluminum; alternatively, triethylaluminum; alternatively, tripropylaluminum; alternatively, tri-n-butylaluminum; alternatively, tri-isobutylaluminum; alternatively, tri-n-hexylaluminum; or alternatively, tri-n-octylaluminum.

In a non-limiting embodiment, useful alkylaluminum halides can include diethylaluminum chloride, diethylaluminum bromide, ethylaluminum dichloride, ethylaluminum sesquichloride, and mixtures thereof. In some non-limiting embodiments, useful alkylaluminum halides can include diethylaluminum chloride, ethylaluminum dichloride, ethylaluminum sesquichloride, and mixtures thereof. In other non-limiting embodiments, useful alkylaluminum halides can be diethylaluminum chloride; alternatively, diethylaluminum bromide; alternatively, ethylaluminum dichloride; or alternatively, ethylaluminum sesquichloride.

In one aspect, the present disclosure provides for precontacting the metallocene with at least one organoaluminum compound and an olefin monomer to form a precontacted mixture, prior to contact this precontacted mixture with the solid oxide activator-support to form the active catalyst. When the catalyst system is prepared in this manner, typically, though not necessarily, a portion of the organoaluminum compound can be added to the precontacted mixture and another portion of the organoaluminum compound can be added to the postcontacted mixture prepared when the precontacted mixture can be contacted with the solid oxide activator. However, all the organoaluminum compound can be used to prepare the catalyst system in either the precontacting or postcontacting step. Alternatively, all the catalyst system components can be contacted in a single step.

Further, more than one organoaluminum compounds can be used, in either the precontacting or the postcontacting step. When an organoaluminum compound is added in multiple steps, the amounts of organoaluminum compound disclosed herein include the total amount of organoaluminum compound used in both the precontacted and postcontacted mixtures, and any additional organoaluminum compound added to the oligomerization reactor. Therefore, total amounts of organoaluminum compounds are disclosed, regardless of whether a single organoaluminum compound is used, or more than one organoaluminum compound. In another aspect, triethylaluminum (TEA) or triisobutylaluminum are typical organoaluminum compounds used in this disclosure. In some embodiments, the organoaluminum compound can be triethylaluminum; or alternatively, triisobutylaluminum.

In one aspect and in any embodiment disclosed herein wherein the catalyst system utilizes an organoaluminum compound, the molar ratio aluminum of the organoaluminum compound to the metal of the metallocene (Al:metal of the metallocene) can be greater than 0.1:1; alternatively, greater than 1:1; or alternatively, greater than 10:1; or alternatively, greater than 50:1. In some embodiments wherein the catalyst system utilizes an organoaluminum compound, the molar ratio aluminum of the organoaluminum compound to the metal of the metallocene (Al:metal of the metallocene) can range from 0.1:1 to 100,000:1; alternatively, range from 1:1 to 10,000:1; alternatively, range from 10:1 to 1,000:1; or alternatively, range from 50:1 to 500:1. When the metallocene contains a specific metal (e.g. Zr) the molar ratio can be stated as an Al:specific metal molar ratio (e.g. Al:Zr molar ratio).

In another aspect and in any embodiment disclosed herein wherein the catalyst system utilizes an organoaluminum compound, the molar ratio of the aluminum-carbon bonds of the organoaluminum compound to the metal of the metallocene (Al—C bonds:metal of the metallocene) can be greater than 0.1:1; alternatively, greater than 1:1; or alternatively, greater than 10:1; or alternatively; greater than 50:1. In some embodiments wherein the catalyst system utilizes an organoaluminum compound, the molar ratio of the aluminum-carbon bonds or the organoaluminum compound to the metal of the metallocene (Al—C bonds:metal of the metallocene) can range from 0.1:1 to 100,000:1; alternatively, range from 1:1 to 10,000:1; alternatively, range from 10:1 to 1,000:1; or alternatively, range from 50:1 to 500:1. When the metallocene contains a specific metal (e.g. Zr) the ratio can be stated as an Al—C bonds:specific metal ratio (e.g. Al—C bonds:Zr molar ratio).

### The Organozinc Compound

One aspect of this disclosure provides for a method of producing an olefin wax oligomer and/or olefin wax oligomer composition comprising contacting an olefin wax and a catalyst system, wherein the catalyst system can comprise a metallocene and an activator. In an embodiment, the activator can comprise, consist of, or consist essentially of an organozinc compound. The organozinc compound can be used alone or in combination with any other activators disclosed herein. In an aspect of any embodiment provided here, for example, the catalyst system can comprise at least one organozinc compound as an activator, either alone or in combination with a chemically-treated solid oxide, an aluminoxane, or any other activators(s). In some embodiments, the catalyst system can, comprise, consist essentially of, or consist of, a metallocene, a first activator comprising a chemically-treated solid oxide, and a second activator comprising an organozinc compound.

In an aspect, the organozinc compounds that can be used in the catalyst system of this disclosure include but are not limited to compounds having the formula:

Zn(X⁴⁰)ₚ(X⁴¹)₂₋ₚ.

In an embodiment, each X⁴⁰ can be independently a C₁ to C₂₀ hydrocarbyl group; alternatively, a C₁ to C₁₀ hydrocarbyl group; alternatively, a C₆ to C₂₀ aryl group; alternatively, a C₆ to C₁₀ aryl group; alternatively, a C₁ to C₂₀ alkyl group; alternatively, a C₁ to C₁₀ alkyl group; or alternatively, a C₁ to C₅ alkyl group. In an embodiment, each X⁴¹ can be independently a halide, a hydride, or a C₁ to C₂₀ hydrocarboxide group; alternatively, a halide, a hydride, or a C₁ to C₁₀ hydrocarboxide group; alternatively, a halide, a hydride, or a C₆ to C₂₀ aryloxide group; alternatively, a halide, a hydride, or a C₆ to C₁₀ aryloxide group; alternatively, a halide, a hydride, or a C₁ to C₂₀ alkoxide group; alternatively, a halide, a hydride, or a C₁ to C₁₀ alkoxide group; alternatively, a halide, a hydride, or, or a C₁ to C₅ alkoxide group; alternatively, a halide; alternatively, a hydride; alternatively, a C₁ to C₂₀ hydrocarboxide group; alternatively, a C₁ to C₁₀ hydrocarboxide group; alternatively, a C₆ to C₂₀ aryloxide group; alternatively, a C₆ to C₁₀ aryloxide group; alternatively, a C₁ to C₂₀ alkoxide group; alternatively, a C₁ to C₁₀ alkoxide group; alternatively, a C₁ to C₅ alkoxide group. In an embodiment, p can be a number (whole or otherwise) from 1 to 2, inclusive; alternatively, 1; or alternatively, 2. Alkyl groups, aryl groups, alkoxide groups, aryloxide groups, and halides have been independently described herein potential group for X¹⁰ and X¹¹ of the organoaluminum compound having the formula Al(X¹⁰)ₙ(X¹¹)₃₋ₙ and these alkyl groups, aryl groups, alkoxide groups, aryloxide groups, and halides can be utilized without limitation to describe the organozinc compounds having the formula Zn(X⁴⁰)ₚ(X⁴¹)₂₋ₚ that can be used in the aspects and embodiments described in this disclosure.\

In another aspect an in any embodiment of this disclosure, useful organozinc compounds can comprise, consist essentially of, or consist of, dimethylzinc, diethylzinc, dipropylzinc, dibutylzinc, dineopentylzinc, di(trimethylsilylmethyl)zinc, any combinations thereof; alternatively, dimethylzinc; alternatively, diethylzinc; alternatively, dipropylzinc; alternatively, dibutylzinc; alternatively, dineopentylzinc; or alternatively, di(trimethylsilylmethyl)zinc.

In one aspect and in any embodiment disclosed herein wherein the catalyst system utilizes an organozinc compound, the molar ratio of the organozinc compound to the metal of the metallocene (Zn:metal of the metallocene) can be greater than 0.1:1; alternatively, greater than 1:1; or alternatively, greater than 10:1; or alternatively, greater than 50:1. In some embodiments wherein the catalyst system utilizes an organozinc compound, the molar ratio of the organozinc compound to the metal of the metallocene (Zn:metal of the metallocene) can range from 0.1:1 to 100,000:1; alternatively, range from 1:1 to 10,000:1; alternatively, range from 10:1 to 1,000:1; or alternatively, range from 50:1 to 500:1. When the metallocene contains a specific metal (e.g. Zr) the ratio may be stated as a Zn:specific metal ratio (e.g. Zn:Zr molar ratio).

### Organomagnesium Compounds

One aspect of this disclosure provides for a method of producing an olefin wax oligomer and/or an olefin wax oligomer composition comprising contacting an olefin wax and a catalyst system, wherein the catalyst system can comprise a metallocene and an activator. In an embodiment, the activator can comprise, consist of, or consist essentially of, an organomagnesium compound. The organomagnesium compound can be used alone or in combination with any other activators disclosed herein. In an aspect of any embodiment provided here, for example, the catalyst system can comprise at least one organomagnesium compound as an activator, either alone or in combination with a chemically-treated solid oxide, an aluminoxane, or any other activators(s). In some embodiments, the catalyst system can comprise, consist essentially of, or consist of, a metallocene, a first activator comprising a chemically-treated solid oxide, and a second activator comprising an organomagnesium compound.

In an aspect, the organomagnesium compounds that can be used in the catalyst system of this disclosure include but are not limited to compounds having the formula.

Mg(X¹⁷)_{q}(X¹⁸)_{2-q}.

In an embodiment, each X¹⁷ can be independently a C₁ to C₂₀ hydrocarbyl group; alternatively, a C₁ to C₁₀ hydrocarbyl group; alternatively, a C₆ to C₂₀ aryl group; alternatively, a C₆ to C₁₀ aryl group; alternatively, a C₁ to C₂₀ alkyl group; alternatively, a C₁ to C₁₀ alkyl group; or alternatively, a C₁ to C₅ alkyl group. In an embodiment, each X¹⁸ can be independently a halide, a hydride, or a C₁ to C₂₀ hydrocarboxide group; alternatively, a halide, a hydride, or a C₁ to C₁₀ hydrocarboxide group; alternatively, a halide, a hydride, or a C₆ to C₂₀ aryloxide group; alternatively, a halide, a hydride, or a C₆ to C₁₀ aryloxide group; alternatively, a halide, a hydride, or a C₁ to C₂₀ alkoxide group; alternatively, a halide, a hydride, or a C₁ to C₁₀ alkoxide group; alternatively, a halide, a hydride, or, or a C₁ to C₅ alkoxide group; alternatively, a halide; alternatively, a hydride; alternatively, a C₁ to C₂₀ hydrocarboxide group; alternatively, a C₁ to Cₜₒ hydrocarboxide group; alternatively, a C₆ to C₂₀ aryloxide group; alternatively, a C₆ to C₁₀ aryloxide group; alternatively, a C₁ to C₂₀ alkoxide group; alternatively, a C₁ to C₁₀ alkoxide group; alternatively, a C₁ to C₅ alkoxide group. In an embodiment, q can be a number (whole or otherwise) from 1 to 2, inclusive; alternatively, 1; or alternatively, 2. Alkyl groups, aryl groups, alkoxide groups, aryloxide groups, and halides have been independently described herein as potential group for X¹⁰ and X¹¹ of the organoaluminum compound having the formula Al(X¹⁰)ₙ(X¹¹)₃₋ₙ and these alkyl groups, aryl groups, alkoxide groups, aryloxide groups, and halides can be utilized without limitation to describe the organomagnesium compounds having the formula Mg(X¹⁷)_{q}(X¹⁸)_{2-q} that can be used in the aspects and embodiments described in this disclosure. As an example, the organomagnesium compound can include or can be selected from dihydrocarbyl magnesium compounds, Grignard reagents, and similar compounds such as alkoxymagnesium alkyl compounds.

In another aspect an in any embodiment of this disclosure, useful organomagnesium compounds can comprise, consist essentially of, or consist of, dimethylmagnesium, diethylmagnesium, dipropylmagnesium, dibutylmagnesium, dineopentylmagnesium, di(trimethylsilylmethyl)magnesium, methylmagnesium chloride, ethylmagnesium chloride, propylmagnesium chloride, butylmagnesium chloride, neopentylmagnesium chloride, trimethylsilylmethylmagnesium chloride, methylmagnesium bromide, ethylmagnesium bromide, propylmagnesium bromide, butylmagnesium bromide, neopentylmagnesium bromide, trimethylsilylmethylmagnesium bromide, methylmagnesium iodide, ethylmagnesium iodide, propylmagnesium iodide, butylmagnesium iodide, neopentylmagnesium iodide, trimethylsilylmethylmagnesium iodide, methylmagnesium ethoxide, ethylmagnesium ethoxide, propylmagnesium ethoxide, butylmagnesium ethoxide, neopentylmagnesium ethoxide, trimethylsilylmethylmagnesium ethoxide, methylmagnesium propoxide, ethylmagnesium propoxide, propylmagnesium propoxide, butylmagnesium propoxide, neopentylmagnesium propoxide, trimethylsilylmethylmagnesium propoxide, methylmagnesium phenoxide, ethylmagnesium phenoxide, propylmagnesium phenoxide, butylmagnesium phenoxide, neopentylmagnesium phenoxide, trimethylsilylmethylmagnesium phenoxide, any combinations thereof; alternatively, dimethylmagnesium; alternatively, diethylmagnesium; alternatively, dipropylmagnesium; alternatively, dibutylmagnesium; alternatively, dineopentylmagnesium; alternatively, di(trimethylsilylmethyl)magnesium; alternatively, methylmagnesium chloride; alternatively, ethylmagnesium chloride; alternatively, propylmagnesium chloride; alternatively, butylmagnesium chloride; alternatively, neopentylmagnesium chloride; alternatively, trimethylsilylmethylmagnesium chloride; alternatively, methylmagnesium bromide; alternatively, ethylmagnesium bromide; alternatively, propylmagnesium bromide; alternatively, butylmagnesium bromide; alternatively, neopentylmagnesium bromide; alternatively, trimethylsilylmethylmagnesium bromide; alternatively, methylmagnesium iodide; alternatively, ethylmagnesium iodide; alternatively, propylmagnesium iodide; alternatively, butylmagnesium iodide; alternatively, neopentylmagnesium iodide; alternatively, trimethylsilylmethylmagnesium iodide; alternatively, methylmagnesium ethoxide; alternatively, ethylmagnesium ethoxide; alternatively, propylmagnesium ethoxide; alternatively, butylmagnesium ethoxide; alternatively, neopentylmagnesium ethoxide; alternatively, trimethylsilylmethylmagnesium ethoxide; alternatively, methylmagnesium propoxide; alternatively, ethylmagnesium propoxide; alternatively, propylmagnesium propoxide; alternatively, butylmagnesium propoxide; alternatively, neopentylmagnesium propoxide; alternatively, trimethylsilylmethylmagnesium propoxide; alternatively, methylmagnesium phenoxide; alternatively, ethylmagnesium phenoxide; alternatively, propylmagnesium phenoxide; alternatively, butylmagnesium phenoxide; alternatively, neopentylmagnesium phenoxide; or alternatively, trimethylsilylmethylmagnesium phenoxide.

In one aspect and in any embodiment disclosed herein wherein the catalyst system utilizes an organomagnesium compound, the molar ratio of the organomagnesium compound to the metal of the metallocene (Mg:metal of the metallocene) can be greater than 0.1:1; alternatively, greater than 1:1; or alternatively, greater than 10:1; or alternatively, greater than 50:1. In some embodiments wherein the catalyst system utilizes an organomagnesium compound, the molar ratio of the organomagnesium compound to the metal of the metallocene (Mg:metal of the metallocene) can range from 0.1:1 to 100,000:1; alternatively, range from 1:1 to 10,000:1; alternatively, range from 10:1 to 1,000:1; or alternatively, range from 50:1 to 500:1. When the metallocene contains a specific metal (e.g. Zr) the ratio can be stated as a Mg:specific metal ratio (e.g Mg:Zr molar ratio).

### Organolithium Compounds

One aspect of this disclosure provides for a method of producing an olefin wax oligomer and/or an olefin wax oligomer composition comprising contacting an olefin wax and a catalyst system, wherein the catalyst system can comprise a metallocene and an activator. In an embodiment, the activator can comprise, consist of, or consist essentially of, an organolithium compound. The organolithium compound can be used alone or in combination with any other activators disclosed herein. In an aspect of any embodiment provided here, for example, the catalyst system can comprise at least one organolithium compound as an activator, either alone or in combination with a chemically-treated solid oxide, an aluminoxane, or any other activators(s). In some embodiments, the catalyst system can comprise, consist essentially of, or consist of, a metallocene, a first activator comprising a chemically-treated solid oxide, and a second activator comprising an organolithium compound.

In an aspect, the organolithium compounds that can be used in the catalyst system of this disclosure include but are not limited to compounds having the formula:

Li(X¹⁹).

In an embodiment, X¹⁹ can be a C₁ to C₂₀ hydrocarbyl group or hydride; alternatively, a C₁ to C₁₀ hydrocarbyl group; alternatively, a C₆ to C₂₀ aryl group; alternatively, a C₆ to C₁₀ aryl group; alternatively, a C₁ to C₂₀ alkyl group; alternatively, a C₁ to C₁₀ alkyl group; alternatively, a C₁ to C₅ alkyl group; or alternatively, hydride. Alkyl groups and aryl groups have been independently described herein as potential group for X¹⁰ and X¹¹ of the organoaluminum compound having the formula Al(X¹⁰)ₙ(X¹¹)₃₋ₙ and these alkyl groups and aryl groups can be utilized without limitation to describe the organolithium compounds having the formula Li(X¹⁹) that can be used in the aspects and embodiments described in this disclosure.

In another aspect an in any embodiment of this disclosure, useful organolithium compound can comprise, consist essentially of, or consist of, methyllithium, ethyllithium, propyllithium, n-butyllithium, sec-butyllithium, t-butyllithium, neopentyllithium, trimethylsilylmethyllithium, phenyllithium, tolyllithium, xylyllithium, benzyllithium, (dimethylphenyl)methyllithium, allyllithium, or combinations thereof. In an embodiment, the organolithium compound can comprise, consist essentially of, or consist of, methyllithium, ethyllithium, propyllithium, n-butyllithium, sec-butyllithium, t-butyllithium, or any combination thereof; alternatively, phenyllithium, tolyllithium, xylyllithium, or any combination thereof. In some embodiments, the organolithium compound can comprise, consist essentially of, or consist of, methyllithium; alternatively, ethyllithium; alternatively, propyllithium; alternatively, n-butyllithium; alternatively, sec-butyllithium; alternatively, t-butyllithium; alternatively, neopentyllithium; alternatively, trimethylsilylmethyllithium; alternatively, phenyllithium; alternatively, tolyllithium; alternatively, xylyllithium; alternatively, benzyllithium; alternatively, (dimethylphenyl)methyllithium; or alternatively, allyllithium.

In one aspect and in any embodiment disclosed herein wherein the catalyst system utilizes an organolithium compound, the molar ratio of the organolithium compound to the metal of the metallocene (Li:metal of the metallocene) can be greater than 0.1:1; alternatively, greater than 1:1; or alternatively, greater than 10:1; or alternatively, greater than 50:1. In some embodiments wherein the catalyst system utilizes an organolithium compound, the molar ratio of the organolithium compound to the metal of the metallocene (Li:metal of the metallocene) can range from 0.1:1 to 100,000:1; alternatively, range from 1:1 to 10,000:1; alternatively, range from 10:1 to 1,000:1; or alternatively, range from 50:1 to 500:1. When the metallocene contains a specific metal (e.g. Zr) the ratio can be stated as a Li:specific metal ratio (e.g Li:Zr molar ratio).l

### Organoboron Compounds

One aspect of this disclosure provides for a method of producing an olefin wax oligomer and/or an olefin wax oligomer composition comprising contacting an olefin wax and a catalyst system, wherein the catalyst system can comprise a metallocene and an activator. In an embodiment, the activator can comprise, consist of, or consist essentially of, an organoboron compound. The organoboron compound can be used alone or in combination with any other activators disclosed herein. In an aspect of any embodiment provided here, for example, the catalyst system can comprise at least one organoboron compound as an activator, either alone or in combination with a chemically-treated solid oxide, an aluminoxane, or any other activators(s). In some embodiments, the catalyst system can, comprise, consist essentially of, or consist of, a metallocene, a first activator comprising a chemically-treated solid oxide, and a second activator comprising an organoboron compound.

In an aspect, organoboron compounds that can be used in the catalyst system of this disclosure are varied. In one aspect, the organoboron compound can comprise neutral boron compounds, borate salts, or combinations thereof; alternatively, neutral organoboron compound; or alternatively, borate salts. In an aspect, the organoboron compounds of this disclosure can comprise a fluoroorganoboron compound, a fluoroorganoborate compound, or a combination thereof; alternatively, a fluoroorganoboron compound; or alternatively, a fluoroorganoborate compound. Any fluoroorganoboron or fluoroorganoborate compound known in the art can be utilized. The term fluoroorganoboron compound has its usual meaning to refer to neutral compounds of the form BY₃. The term fluoroorganoborate compound also has its usual meaning to refer to the monoanionic salts of a fluoroorganoboron compound of the form [cation]⁺[BY₄]⁻, where Y represents a fluorinated organic group. For convenience, fluoroorganoboron and fluoroorganoborate compounds are typically referred to collectively by organoboron compounds, or by either name as the context requires.

According to one aspect, organoboron compounds that can be used in the catalyst system of this disclosure include but are not limited to compounds having the formula:

B(X⁴²)ₙ(X⁴³)₃₋ₙ.

In an embodiment, each X⁴² can be independently a C₁ to C₂₀ hydrocarbyl group; alternatively, a C₁ to C₁₀ hydrocarbyl group; alternately, a C₆ to C₂₀ aryl group; alternatively, a C₆ to C₁₀ aryl group; alternatively, a C₁ to C₂₀ alkyl group; alternatively, a C₁ to C₁₀ alkyl group; or alternatively, a C₁ to C₅ alkyl group. In an embodiment, each X⁴³ can be independently a halide, a hydride, or a C₁ to C₂₀ hydrocarboxide group (also referred to as a hydrocarboxy group); alternatively, a halide, a hydride, or a C₁ to C₁₀ hydrocarboxide group; alternatively, a halide, a hydride, or a C₆ to C₂₀ aryloxide group (also referred to as an aroxide or aroxy group); alternatively, a halide, a hydride, or a C₆ to C₁₀ aryloxide group; alternatively, a halide, a hydride, or a C₁ to C₂₀ alkoxide group (also referred to as an alkoxy group); alternatively, a halide, a hydride, or a C₁ to C₁₀ alkoxide group; alternatively, a halide, a hydride, or, or a C₁ to C₅ alkoxide group; alternatively, a halide; alternatively, a hydride; alternatively, a C₁ to C₂₀ hydrocarboxide group; alternatively, a C₁ to C₁₀ hydrocarboxide group; alternatively, a C₆ to C₂₀ aryloxide group; alternatively, a C₆ to C₁₀ aryloxide group; alternatively, a C₁ to C₂₀ alkoxide group; alternatively, a C₁ to C₁₀ alkoxide group; alternatively, a C₁ to C₅ alkoxide group. In an embodiment, n can be a number (whole or otherwise) from 1 to 3, inclusive; alternatively, about 1.5, alternatively, or alternatively, 3.

In an embodiment, each alkyl group(s) of the organoboron compound having the formula B(X⁴²)ₙ(X⁴³)₃₋ₙ can be independently a methyl group, an ethyl group, a propyl group, a butyl group, a pentyl group, a hexyl group, a heptyl group, or an octyl group; alternatively, a methyl group, a ethyl group, a butyl group, a hexyl group, or an octyl group. In some embodiments, each alkyl group(s) of the organoboron compound having the formula B(X⁴²)ₙ(X⁴³)₃₋ₙ can be independently a methyl group, an ethyl group, an n-propyl group, an n-butyl group, an iso-butyl group, a n-hexyl group, or an n-octyl group; alternatively, a methyl group, an ethyl group, a n-butyl group, or an iso-butyl group; alternatively, a methyl group; alternatively, an ethyl group; alternatively, an n-propyl group; alternatively, an n-butyl group; alternatively, an iso-butyl group; alternatively, a n-hexyl group; or alternatively, an n-octyl group. In an embodiment, each aryl group of the organoboron compound having the formula B(X⁴²)ₙ(X⁴³)₃₋ₙ can be independently a phenyl group or a substituted phenyl group; alternatively, a phenyl group; or alternatively, a substituted phenyl group. Substituted phenyl groups are described herein and these substituted phenyl group may be utilized without limitation for the organoboron compound having the formula B(X⁴²)ₙ(X⁴³)₃₋ₙ.

In an embodiment, each halide of the organoboron compound having the formula B(X⁴²)ₙ(X⁴³)₃₋ₙ can be independently a fluoride, chloride, bromide, or iodide. In some embodiments, each halide of the organoboron compound having the formula B(X⁴²)ₙ(X⁴³)₃₋ₙ can be independently a fluoride; alternatively, chloride; alternatively, bromide; or alternatively, iodide.

In an embodiment, each alkoxide of the organoboron compound having the formula B(X⁴²)ₙ(X⁴³)₃₋ₙ can be independently a methoxy group, an ethoxy group, a propoxy group, a butoxy group, a pentoxy group, a hexoxy group, a heptoxy group, or an octoxy group; alternatively, a methoxy group, a ethoxy group, a butoxy group, a hexoxy group, or an octoxy group. In some embodiments, the alkoxy group can be independently a methoxy group, an ethoxy group, an n-propoxy group, an n-butoxy group, an iso-butoxy group, a n-hexoxy group, or an n-octoxy group; alternatively, a methoxy group, an ethoxy group, a n-butoxy group, or an iso-butoxy group; alternatively, a methoxy group; alternatively, an ethoxy group; alternatively, an n-propoxy group; alternatively, an n-butoxy group; alternatively, an iso-butoxy group; alternatively, a n-hexoxy group; or alternatively, an n-octoxy group. In an embodiment, each aryloxide of the organoboron compound having the formula B(X⁴²)ₙ(X⁴³)₃₋ₙ can be independently a be a phenoxide or a substituted phenoxide; alternatively, a phenoxide; or alternatively, a substituted phenoxide.

In an embodiment, the organoboron compound that can utilized in any aspect or embodiment of this disclosure can comprise, consist essentially of, or consist of, a trialkylboron, a dialkylaluminium halide, an alkylboron dihalide, a dialkylboron alkoxide, an alkylboron dialkoxide, a dialkylboron hydride, an alkylboron dihydride, or any combination thereof. In other embodiments, the organoboron compound that can utilized in any aspect or embodiment of this disclosure can comprise, consist essentially of, or consist of, a trialkylboron, a dialkylaluminium halide, an alkylboron dihalide, or any combination thereof; alternatively, a trialkylboron; alternatively, a dialkylaluminium halide; alternatively, an alkylboron dihalide; alternatively, a dialkylboron alkoxide; alternatively, an alkylboron dialkoxide; alternatively, a dialkylboron hydride; or alternatively, an alkylboron dihydride. In yet other embodiments, the organoboron compound that that can utilized in any aspect or embodiment of this disclosure can comprise, consist essentially of, or consist of, a trialkylboron, an alkylboron halide, or any combination thereof; alternatively, a trialkylboron; or alternatively, an alkylboron halide.

In a non-limiting embodiment, useful trialkylboron compounds can include trimethylboron, triethylboron, tripropylboron, tributylboron, trihexylboron, trioctylboron, or mixtures thereof. In some non-limiting embodiments, useful trialkylboron compounds can include trimethylboron, triethylboron, tripropylboron, tri-n-butylboron, tri-isobutylboron, trihexylboron, tri-n-octylboron, or mixtures thereof; alternatively, triethylboron, tri-n-butylboron, tri-isobutylboron, tri-n-hexylboron, tri-n-octylboron, or mixtures thereof; alternatively, triethylboron, tri-n-butylboron, tri-n-hexylboron, tri-n-octylboron, or mixtures thereof. In other non-limiting embodiments, useful trialkylboron compounds can be trimethylboron; alternatively, triethylboron; alternatively, tripropylboron; alternatively, tri-n-butylboron; alternatively, tri-isobutylboron; alternatively, tri-n-hexylboron; or alternatively, tri-n-octylboron.

In an embodiment, the fluoroorganoborate compounds that can be used as activators in the present disclosure include, but are not limited to, fluorinated aryl borates such as, N,N-dimethylanilinium tetrakis(pentafluorophenyl)borate, triphenylcarbenium tetrakis(pentafluorophenyl)borate, lithium tetrakis(pentafluorophenyl)borate, N,N-dimethylanilinium tetrakis[3,5-bis(trifluoromethyl)phenyl]borate, triphenylcarbenium tetrakis[3,5-bis(trifluoromethyl)-phenyl]borate, and mixtures thereof; alternatively, N,N-dimethylanilinium tetrakis(pentafluorophenyl)-borate; alternatively, triphenylcarbenium tetrakis(pentafluorophenyl)borate; alternatively, lithium tetrakis-(pentafluorophenyl)borate; alternatively, N,N-dimethylanilinium tetrakis[3,5-bis(trifluoro-methyl)phenyl]borate; or alternatively, triphenylcarbenium tetrakis[3,5-bis(trifluoromethyl)phenyl]borate. Examples of fluoroorganoboron compounds that can be used as activators in the present disclosure include, but are not limited to, tris(pentafluorophenyl)boron, tris[3,5-bis(trifluoromethyl)phenyl]boron, and mixtures thereof.

Although not intending to be bound by the following theory, these fluoroorganoborate and fluoroorganoboron compounds, and related compounds, are thought to form “weakly-coordinating” anions when combined with organometal compounds, as disclosed in U.S. Pat. No. 5,919,983, which is incorporated herein by reference in its entirety.

Generally, any amount of organoboron compound can be utilized in this disclosure. In one aspect and in any embodiment disclosed herein, the molar ratio of the organoboron compound to the metallocene can be from 0.001:1 to 100,000:1. Alternatively and in any embodiment, the molar ratio of the organoboron compound to the metallocene can be from 0.01:1 to 10,000:1; alternatively, from 0.1:1 to 100:1; alternatively, from 0.5:1 to 10:1; or alternatively, from 0.2:1 to 5:1. Typically, the amount of the fluoroorganoboron or fluoroorganoborate compound used as an activator for the metallocenes can be in a range of from 0.5 mole to 10 moles of organoboron compound per total mole of metallocene compounds employed. In one aspect, the amount of fluoroorganoboron or fluoroorganoborate compound used as an activator for the metallocene is in a range of 0.8 mole to 5 moles of organoboron compound per total moles of metallocene compound.

### Ionizing Ionic Compounds

One aspect of this disclosure provides for a method of producing an olefin wax oligomer and/or an olefin wax oligomer composition comprising contacting an olefin wax and a catalyst system, wherein the catalyst system can comprise a metallocene and an activator. In an embodiment, the activator can comprise, consist of, or consist essentially of, an ionizing ionic compound. The ionizing ionic compound can be used alone or in combination with any other activators disclosed herein. In an aspect of any embodiment provided here, for example, the catalyst system can comprise at least one ionizing ionic compound as an activator, either alone or in combination with a chemically-treated solid oxide, an aluminoxane, or any other activators(s). In some embodiments, the catalyst system can comprise, consist essentially of, or consist of, a metallocene, a first activator comprising a chemically-treated solid oxide, and a second activator comprising an ionizing ionic compound. Examples of ionizing ionic compound are disclosed in U.S. Pat. Nos. 5,576,259 and 5,807,938, each of which is incorporated herein by reference, in its entirety.

An ionizing ionic compound is an ionic compound which can function to enhance the activity of the catalyst system. While not bound by theory, it is believed that the ionizing ionic compound can be capable of reacting with the metallocene compound and converting it into a cationic metallocene compound or a metallocene compound that can be an incipient cation. Again, while not intending to be bound by theory, it is believed that the ionizing ionic compound can function as an ionizing compound by at least partially extracting an anionic ligand, possibly a Group II (non-η⁵-alkadienyl) ligand from the metallocenes. However, the ionizing ionic compound is an activator regardless of whether it is ionizes the metallocenes, abstracts a Group II ligand in a fashion as to form an ion pair, weakens the metal-Group II ligand bond in the metallocene, simply coordinates to a Group II ligand, or any other mechanisms by which activation may occur.

Further, it is not necessary that the ionizing ionic compound activate the metallocenes only. The activation function of the ionizing ionic compound may be evident in the enhanced activity of the catalyst system as a whole, as compared to a catalyst system that does not comprise any ionizing ionic compound. It is also not necessary that the ionizing ionic compound activate different metallocenes to the same extent.

In one aspect and in any embodiment disclosed herein, the ionizing ionic compound can have the formula:

[Q]⁺[M⁴Z₄]⁻.

In an embodiment, Q is can be [NR^{A}R^{B}R^{C}R^{D}]⁺, [CR^{E}R^{F}R^{G}]⁺, [C₇H₇]⁺, Li⁺, Na⁺, or K⁺; alternatively, [NR^{A}R^{B}R^{C}R^{D}]⁺; alternatively, [CR^{E}R^{F}R^{G}]⁺; alternatively, [C₇H₇]⁺; alternatively, Li⁺, Na⁺, or K⁺; alternatively, Li⁺; alternatively, Na⁺; or alternatively, K. In an embodiment, R^{A}, R^{B}, and R^{C} can each independently be a hydrogen, or a C₁ to C₂₀ hydrocarbyl group; alternatively, hydrogen or a C₁ to C₁₀ hydrocarbyl group; alternatively, hydrogen or a C₁ to C₅ hydrocarbyl group; alternatively, hydrogen or a C₆ to C₂₀ aryl group; alternatively, hydrogen or a C₆ to C₁₅ aryl group; alternatively, hydrogen or a C₆ to C₁₀ aryl group; alternatively, hydrogen or a C₁ to C₂₀ alkyl group; alternatively, hydrogen or a C₁ to C₁₀ alkyl group; or alternatively, hydrogen or a C₁ to C₅ alkyl group; alternatively, hydrogen; alternatively, a C₁ to C₂₀ hydrocarbyl group; alternatively, a C₁ to C₁₀ hydrocarbyl group; alternatively, a C₁ to C₅ hydrocarbyl group; alternatively, a C₆ to C₂₀ aryl group; alternatively, a C₆ to C₁₅ aryl group; alternatively, C₆ to C₁₀ aryl group; alternatively, a C₁ to C₂₀ alkyl group; alternatively, a C₁ to C₁₀ alkyl group; or alternatively, a C₁ to C₅ alkyl group. In an embodiment, R^{D} can be hydrogen, a halide, or a C₁ to C₂₀ hydrocarbyl group; alternatively, hydrogen, a halide, or a C₁ to C₁₀ hydrocarbyl group; alternatively, hydrogen, a halide, or a C₁ to C₅ hydrocarbyl group; alternatively, hydrogen, a halide, or a C₆ to C₂₀ aryl group; alternatively, hydrogen, a halide, or a C₆ to C₁₅ aryl group; alternatively, hydrogen, a halide, or a C₆ to C₁₀ aryl group; alternatively, hydrogen, a halide, or a C₁ to C₂₀ alkyl group; alternatively, hydrogen, a halide, or a C₁ to C₁₀ alkyl group; or alternatively, hydrogen, a halide, or a C₁ to C₅ alkyl; alternatively, hydrogen; alternatively, a halide; alternatively, or a C₁ to C₂₀ hydrocarbyl group; alternatively, a C₁ to C₁₀ hydrocarbyl group; alternatively, a C₁ to C₅ hydrocarbyl group; alternatively, a C₆ to C₂₀ aryl group; alternatively, a C₆ to C₁₅ aryl group; alternatively, a C₆ to C₁₀ aryl group; alternatively, a C₁ to C₂₀ alkyl group; alternatively, a C₁ to C₁₀ alkyl group; or alternatively, a C₁ to C₅ alkyl group. In an embodiment, R^{E}, R^{F}, and R^{G} can each independently be hydrogen, a halide, or a C₁ to C₂₀ hydrocarbyl group; alternatively, hydrogen, a halide, or a C₁ to C₁₀ hydrocarbyl group; alternatively, hydrogen, a halide, or a C₁ to C₅ hydrocarbyl group; alternatively, hydrogen, a halide, or a C₆ to C₂₀ aryl group; alternatively, hydrogen, a halide, or a C₆ to C₁₅ aryl group; or alternatively, hydrogen, a halide, or a C₆ to C₁₀ aryl group; alternatively, hydrogen; alternatively, a halide; alternatively, a C₁ to C₂₀ hydrocarbyl group; alternatively, a C₁ to C₁₀ hydrocarbyl group; alternatively, a C₁ to C₅ hydrocarbyl group; alternatively, a C₆ to C₂₀ aryl group; alternatively, a C₆ to C₁₅ aryl group; or alternatively, a C₆ to C₁₀ aryl group. Alkyl groups, aryl groups, and halides have been independently described herein potential group for X¹⁰ and X¹¹ of the organoaluminum compound having the formula Al(X¹⁰)ₙ(X¹¹)₃₋ₙ and these alkyl groups, aryl groups, and halides can be utilized without limitation to describe the ionizing ionic compound having the formula [NR^{A}R^{B}R^{C}R^{D}]⁺ or [CR^{E}R^{F}R^{G}]⁺ that can be used in the aspects and embodiments described in this disclosure.

In some embodiments, Q can be a trialkyl ammonium or a dialkylarylammonium (e.g. dimethyl anilinium); alternatively, triphenylcarbenium or substituted triphenylcarbenium; alternatively, tropylium or a substituted tropylium; alternatively, a trialkylammonium; alternatively, a dialkylarylammonium (e.g. dimethyl anilinium); alternatively, a triphenylcarbenium; or alternatively, tropylium. In other embodiments, Q can be tri(n-butyl)ammonium, N,N-dimethylanilinium, triphenylcarbenium, tropylium, lithium, sodium, and potassium; alternatively, tri(n-butyl)ammonium and N,N-dimethylanilinium; alternatively, triphenylcarbenium, tropylium; or alternatively, lithium, sodium and potassium. In an embodiment, M⁴ can be B or Al; alternatively, B; or alternatively, Al. In an embodiment, Z can be halide or

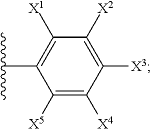

alternatively, halide; or alternatively,

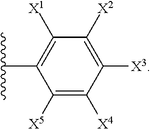

In an embodiment, X¹, X², X³, X⁴, and X⁵ can be independently hydrogen, a halide, a C₁ to C₂₀ hydrocarbyl group, or a C₁ to C₂₀ hydrocarboxide group (also referred to herein as a hydrocarboxy group); alternatively, hydrogen, a halide, a C₁ to C₁₀ hydrocarbyl group, or a C₁ to C₁₀ hydrocarboxide group; alternatively, hydrogen, a halide, a C₆ to C₂₀ aryl group, or a C₆ to C₂₀ aryloxide group; alternatively, hydrogen, a halide, a C₆ to C₁₀ aryl group, or a C₆ to C₁₀ aryloxide group; alternatively, hydrogen, a halide, a C₁ to C₂₀ alkyl group, or a C₁ to C₂₀ alkoxide group (also referred to herein as an alkoxy group); alternatively, hydrogen, a halide, a C₁ to C₁₀ alkyl group, or a C₁ to C₁₀ alkoxide group; or alternatively, hydrogen, a halide, a C₁ to C₅ alkyl group, or a C₁ to C₅ alkoxide group. Alkyl groups, aryl groups, alkoxide groups, aryloxide groups, and halides have been independently described herein potential group for X¹⁰ and X¹¹ of the organoaluminum compound having the formula Al(X¹⁰)ₙ(X¹¹)₃₋ₙ and these alkyl groups, aryl groups, alkoxide groups, aryloxide groups, and halides can be utilized without limitation as X¹, X², X³, X⁴, and X⁵. In some embodiments,

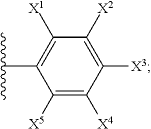

can be phenyl, p-tolyl, m-tolyl, 2,4-dimethylphenyl, 3,5-dimethylphenyl, pentafluorophenyl, and 3,5-bis(trifluoromethyl)phenyl; alternatively, phenyl; alternatively, p-tolyl; alternatively, m-tolyl; alternatively, 2,4-dimethylphenyl; alternatively, 3,5-dimethylphenyl; alternatively, pentafluorophenyl; or alternatively, 3,5-bis(trifluoro-methyl)phenyl.

Examples of ionizing ionic compounds include, but are not limited to, the following compounds: tri(n-butyl)ammonium tetrakis(p-tolyl)borate, tri(n-butyl)ammonium tetrakis(m-tolyl)borate, tri(n-butyl)ammonium tetrakis(2,4-dimethylphenyl)borate, tri(n-butyl)ammonium tetrakis(3,5-dimethylphenyl)borate, tri(n-butyl)ammonium tetrakis[3,5-bis(trifluoromethyl)phenyl]borate, tri(n-butyl)ammonium tetrakis(pentafluorophenyl)borate, N,N-dimethylanilinium tetrakis(p-tolyl)borate, N,N-dimethylanilinium tetrakis(m-tolyl)borate, N,N-dimethylanilinium tetrakis(2,4-dimethylphenyl)borate, N,N-dimethylanilinium tetrakis(3,5-dimethylphenyl)borate, N,N-dimethylanilinium tetrakis[3,5-bis(trifluoromethyl)phenyl]borate, or N,N-dimethylanilinium tetrakis(pentafluorophenyl)borate; alternatively, triphenylcarbenium tetrakis(p-tolyl)borate, triphenylcarbenium tetrakis(m-tolyl)borate, triphenylcarbenium tetrakis(2,4-dimethylphenyl)borate, triphenylcarbenium tetrakis(3,5-dimethylphenyl)borate, triphenylcarbenium tetrakis[3,5-bis(trifluoromethyl)phenyl]borate, or triphenylcarbenium tetrakis(pentafluorophenyl)borate; alternatively, tropylium tetrakis(p-tolyl)borate, tropylium tetrakis(m-tolyl)borate, tropylium tetrakis(2,4-dimethylphenyl)borate, tropylium tetrakis(3,5-dimethylphenyl)borate, tropylium tetrakis[3,5-bis(trifluoromethyl)phenyl]borate, or tropylium tetrakis(pentafluorophenyl)borate; alternatively, lithium tetrakis(pentafluorophenyl)borate, lithium tetrakis(phenyl)borate, lithium tetrakis(p-tolyl)borate, lithium tetrakis(m-tolyl)borate, lithium tetrakis(2,4-dimethylphenyl)borate, lithium tetrakis(3,5-dimethylphenyl)borate, or lithium tetrafluoroborate; alternatively, sodium tetrakis(pentafluorophenyl)borate, sodium tetrakis(phenyl)borate, sodium tetrakis(p-tolyl)borate, sodium tetrakis(m-tolyl)borate, sodium tetrakis(2,4-dimethylphenyl)borate, sodium tetrakis(3,5-dimethylphenyl)borate, or sodium tetrafluoroborate; alternatively, potassium tetrakis-(pentafluorophenyl)borate, potassium tetrakis(phenyl)borate, potassium tetrakis(p-tolyl)borate, potassium tetrakis(m-tolyl)borate, potassium tetrakis(2,4-dimethylphenyl)borate, potassium tetrakis(3,5-dimethylphenyl)borate, or potassium tetrafluoroborate; alternatively, tri(n-butyl)ammonium tetrakis(p-tolyl)aluminate, tri(n-butyl)ammonium tetrakis(m-tolyl)aluminate, tri(n-butyl)ammonium tetrakis(2,4-dimethylphenyl)aluminate, tri(n-butyl)ammonium tetrakis(3,5-dimethylphenyl)aluminate, tri(n-butyl)ammonium tetrakis(pentafluorophenyl)aluminate, N,N-dimethylanilinium tetrakis(p-tolyl)-aluminate, N,N-dimethylanilinium tetrakis(m-tolyl)aluminate, N,N-dimethylanilinium tetrakis(2,4-dimethylphenyl)aluminate, N,N-dimethylanilinium tetrakis(3,5-dimethylphenyl)aluminate, or N,N-dimethylanilinium tetrakis(pentafluorophenyl)aluminate; alternatively, triphenylcarbenium tetrakis(p-tolyl)aluminate, triphenylcarbenium tetrakis(m-tolyl)aluminate, triphenylcarbenium tetrakis(2,4-dimethylphenyl)aluminate, triphenylcarbenium tetrakis(3,5-dimethylphenyl)aluminate, or triphenylcarbenium tetrakis(pentafluorophenyl)aluminate; alternatively, tropylium tetrakis(p-tolyl)aluminate, tropylium tetrakis(m-tolyl)aluminate, tropylium tetrakis(2,4-dimethylphenyl)aluminate, tropylium tetrakis(3,5-dimethylphenyl)aluminate, or tropylium tetrakis(pentafluorophenyl)aluminate; alternatively, lithium tetrakis(pentafluorophenyl)aluminate, lithium tetrakis(phenyl)aluminate, lithium tetrakis(p-tolyl)aluminate, lithium tetrakis(m-tolyl)aluminate, lithium tetrakis(2,4-dimethylphenyl)aluminate, lithium tetrakis(3,5-dimethylphenyl)aluminate, or lithium tetrafluoroaluminate; alternatively, sodium tetrakis(pentafluorophenyl)aluminate, sodium tetrakis(phenyl)aluminate, sodium tetrakis(p-tolyl)aluminate, sodium tetrakis(m-tolyl)aluminate, sodium tetrakis(2,4-dimethylphenyl)aluminate, sodium tetrakis(3,5-dimethylphenyl)aluminate, or sodium tetrafluoroaluminate; or alternatively, potassium tetrakis(pentafluorophenyl)aluminate, potassium tetrakis-(phenyl)aluminate, potassium tetrakis(p-tolyl)aluminate, potassium tetrakis(m-tolyl)aluminate, potassium tetrakis(2,4-dimethylphenyl)aluminate, potassium tetrakis(3,5-dimethylphenyl)aluminate, potassium tetrafluoroaluminate. In some embodiments, the ionizing ionic compound can be tri(n-butyl)ammonium tetrakis(3,5-dimethylphenyl)borate, tri(n-butyl)ammonium tetrakis[3,5-bis(trifluoromethyl)phenyl]borate, tri(n-butyl)ammonium tetrakis(pentafluorophenyl)borate, N,N-dimethylanilinium tetrakis(p-tolyl)borate, N,N-dimethylanilinium tetrakis(m-tolyl)borate, N,N-dimethylanilinium tetrakis[3,5-bis(trifluoro-methyl)phenyl]borate, N,N-dimethylanilinium tetrakis(pentafluorophenyl)borate, triphenylcarbenium tetrakis(p-tolyl)borate, triphenylcarbenium tetrakis(m-tolyl)borate, triphenylcarbenium tetrakis(2,4-dimethylphenyl)borate, triphenylcarbenium tetrakis(3,5-dimethylphenyl)borate, triphenylcarbenium tetrakis[3,5-bis(trifluoromethyl)phenyl]borate, lithium tetrakis(p-tolyl)aluminate, lithium tetrakis(m-tolyl)aluminate, lithium tetrakis(2,4-dimethylphenyl)aluminate, or lithium tetrakis(3,5-dimethylphenyl)aluminate.

Alternatively and in some embodiments, the ionizing ionic compound can be tri(n-butyl)-ammonium tetrakis[3,5-bis(trifluoromethyl)phenyl]borate, tri(n-butyl)ammonium tetrakis(pentafluorophenyl)borate, tetrakis[3,5-bis(trifluoromethyl)phenyl]borate, N,N-dimethylanilinium tetrakis(pentafluorophenyl)borate, triphenylcarbenium tetrakis[3,5-bis(trifluoro-methyl)phenyl]borate, lithium tetrakis(p-tolyl)aluminate, or lithium tetrakis(m-tolyl)aluminate; alternatively, tri(n-butyl)ammonium tetrakis[3,5-bis(trifluoromethyl)phenyl]borate; alternatively, tri(n-butyl)ammonium tetrakis(pentafluorophenyl)borate; alternatively, N,N-dimethylanilinium tetrakis[3,5-bis(trifluoromethyl)phenyl]borate; alternatively, N,N-dimethylanilinium tetrakis(pentafluorophenyl)borate; alternatively, triphenylcarbenium tetrakis[3,5-bis(trifluoro-methyl)phenyl]borate; alternatively, lithium tetrakis(p-tolyl)aluminate; or alternatively, lithium tetrakis(m-tolyl)aluminate. In other embodiments, the ionizing compound can be a combination of any ionizing compound recited herein. However, the ionizing ionic compound is not limited thereto in the present disclosure.

In one aspect and in any embodiment disclosed herein, the molar ratio of the ionizing ionic compound to the metallocene can be from 0.001:1 to 100,000:1. Alternatively and in any aspect or embodiment, the molar ratio of the ionizing ionic compound to the metallocene can be from 0.01:1 to 10,000:1; alternatively, from 0.1:1 to 100:1; alternatively, from 0.5:1 to 10:1; or alternatively, from 0.2:1 to 5:1.

### Catalyst System

In an aspect, this disclosure encompasses a catalyst system comprising a metallocene. Generally, the metallocene may be any metallocene described herein.

In an aspect, this disclosure encompasses a catalyst system comprising a metallocene and a chemically-treated solid oxide. The metallocene and chemically-treated solid oxide are independent elements of the catalyst system comprising a metallocene and a chemically-treated solid oxide. Consequently, the metallocene may be any metallocene described herein and the chemically treated solid oxide may be any chemically-treated solid oxide described herein. In an embodiment, the catalyst system comprising a metallocene and a chemically-treated solid oxide may further comprise an activator; alternatively, at least one activator. The activators are independently described herein and may be utilized without limitation to describe further catalyst systems comprising a metallocene and a chemically-treated solid oxide.

In an aspect, this disclosure encompasses a catalyst system comprising a metallocene, a chemically-treated solid oxide, and an organoaluminum compound. Alternatively, this disclosure encompasses a catalyst system consisting essentially of a metallocene, a chemically-treated solid oxide, and an organoaluminum compound. Sometimes, the chemically-treated solid oxide may be referred to as a first activator while the organoaluminum compound may be referred to as a second activator. The metallocene, chemically-treated solid oxide, and organoaluminum compound are independent elements of the catalyst system comprising a metallocene, a chemically-treated solid oxide, and an organoaluminum compound. Consequently, the metallocene may be any metallocene described herein, the chemically treated solid oxide may be any chemically-treated solid oxide described herein, and the organoaluminum compound may be any organoaluminum compound described herein. In an embodiment, the catalyst system comprising a metallocene, a chemically-treated solid oxide, and an organoaluminum compound may further comprise additional activators. These other activators are independently described herein and may be utilized without limitation to describe further catalyst systems comprising a metallocene, a chemically-treated solid oxide, and an organoaluminum compound.

In an aspect, this disclosure encompasses a catalyst system comprising a metallocene and an alumoxane. Alternatively, this disclosure encompasses a catalyst system consisting essentially of a metallocene and an alumoxane. Sometimes, the alumoxane may be referred to as an activator. The metallocene and alumoxane are independent elements of the catalyst system comprising a metallocene and an alumoxane. Consequently, the metallocene may be any metallocene described herein and the alumoxane may be any alumoxane described herein. In an embodiment, the catalyst system comprising a metallocene and an alumoxane may further comprise another activator; alternatively, at least one other activator. These other activators are independently described herein and may be utilized without limitation to describe further catalyst systems comprising a metallocene and an alumoxane.

For illustration purposes, exemplary metallocenes, alumoxanes, organoaluminum compounds, and chemically-treated solid oxides will be provided in this section. However, is not meant to limit the metallocenes, alumoxanes, organoaluminum compounds, and chemically-treated solid oxides which may be utilized in the catalyst systems. Any other metallocene, alumoxane, organoaluminum compound, and chemically-treated solid oxide described herein may be utilized in the catalyst system of this disclosure.

In a non-limiting embodiment, the metallocene may have the formula ZrR¹⁰R¹¹X⁹₂ wherein each X⁹ independently is a halogen atom, R¹⁰ and R¹¹ are substituted or unsubstituted η⁵-indenyl groups, and optionally R¹⁰ and R¹¹ may be connected by a linking group. In an embodiment, X⁹ of the metallocene having the formula ZrR¹⁰R¹¹X⁹₂ may be chlorine; or alternatively bromine. In some embodiments, R¹⁰ and R¹¹ are unsubstituted indenyl groups; alternatively, any substituted indenyl groups disclosed herein. In some particular embodiments, any substituent of the substituted η⁵-indenyl groups may be a C₁-C₂₀ hydrocarbyl group; alternatively, a C₁-C₁₀ hydrocarbyl group; alternatively, a C₁-C₁₀ alkyl group; or alternatively, a C₁-C₅ alkyl group. In some embodiment one of the substituents of a substituted η⁵-indenyl group may be a C₃-C₁₂ alkenyl group. In an embodiment, R¹⁰ and R¹¹, whether substituted or unsubstituted, may be connected by a linking group. In an embodiment, the linking group linking the η⁵-indenyl groups (substituted or unsubstituted) of the metallocene having the formula ZrR¹⁰R¹¹X⁹₂ may have the formula >CR¹R², >SiR³R⁴, or —CR⁵R⁶CR⁷R⁸—, and R¹, R², R³, R⁴, R⁵, R⁶, R⁷, and R⁸ independently are hydrogen or a C₁-C₂₀ hydrocarbyl group; alternatively, hydrogen or a C₁-C₁₀ hydrocarbyl group; alternatively, a hydrogen or a C₁-C₂₀ alkyl group; alternatively, hydrogen or a C₁-C₁₀ alkyl group; or alternatively, hydrogen or a C₁-C₅ alkyl group. In some particular embodiments, at least one of the R groups on the linking group is a C₃-C₁₂ alkenyl group. In an embodiment, the linking group linking the η⁵-indenyl group (substituted or unsubstituted) of the metallocene having the formula ZrR¹⁰R¹¹X⁹₂ may have the formula >CR¹R² and R¹ and R² independently are hydrogen or a C₁-C₂₀ hydrocarbyl group; alternatively, hydrogen or a C₁-C₁₀ hydrocarbyl group; alternatively, a hydrogen or a C₁-C₂₀ alkyl group; alternatively, hydrogen or a C₁-C₁₀ alkyl group; alternatively, hydrogen or a C₁-C₅ alkyl group. In some particular embodiments, at least one of the R¹ or R² is a C₃-C₁₂ alkenyl group. In other embodiments, the linking group has the formula >CR¹R² wherein R¹ is a C₃-C₁₂ alkenyl group and R² is a hydrogen, C₁ to C₂₀ alkyl group or a C₆ to C₂₀ aryl group; R¹ is a C₃-C₁₂ alkenyl group and R² is a hydrogen, C₁ to C₁₀ alkyl group or a C₆ to C₁₀ aryl group; or alternatively, R¹ is a C₃-C₁₂ alkenyl group and R² is a C₁ to C₅ alkyl group or a C₆ to C₁₀ aryl group.

In a non-limiting embodiment, the metallocene may have the formula ZrR¹⁰R¹¹X⁹₂ wherein each X⁹ independently is a halogen atom, R¹⁰ is a substituted or unsubstituted η⁵-cyclopentadienyl group, R¹¹ is a substituted or unsubstituted η⁵-fluorenyl group and R¹⁰ and R¹¹ are connected by a linking group. Within embodiments of the metallocene having the formula ZrR¹⁰R¹¹X⁹₂, X⁹, R¹⁰, and R¹¹ and may be any halide disclosed herein, any substituted or unsubstituted η⁵-cyclopentadienyl group disclosed herein, and any substituted or unsubstituted η⁵-fluorenyl group disclosed herein, respectively. Additionally, the group linking R¹⁰ and R¹¹ may be any linking group disclosed herein.

In an embodiment, X⁹ of the metallocene having the formula ZrR¹⁰R¹¹X⁹₂ may be chlorine; or alternatively bromine. In an embodiment, R¹⁰ of the metallocene having the formula ZrR¹⁰R¹¹X⁹₂ may be an unsubstituted η⁵-cyclopentadienyl group; or alternatively, any substituted η⁵-cyclopentadienyl group disclosed herein.

In an embodiment, and R¹¹ of the metallocene having the formula ZrR¹⁰R¹¹X⁹₂ may be an unsubstituted η⁵-fluorenyl group; or alternatively, any substituted η⁵-cyclopentadienyl group disclosed herein. Generally, the linking group linking the η⁵-fluorenyl group (substituted or unsubstituted) and the η⁵-cyclopentadienyl group (substituted or unsubstituted) is attached at the 9 position of the η⁵-fluorenyl group. In some embodiments, excluding the linking group, the substituted η⁵-fluorenyl group has substituents located at the 2 and 7 positions; alternatively, only has substituents at the 2 and 7 positions. In some particular embodiments, the substituents of the substituted η⁵-cyclopentadienyl group or substituted η⁵-fluorenyl group may be a C₁-C₂₀ hydrocarbyl group; alternatively, a C₁-C₁₀ hydrocarbyl group; alternatively, a C₁-C₁₀ alkyl group; or alternatively, a C₁-C₅ alkyl group; or alternatively, a C₃-C₁₂ alkenyl group. In an embodiment, the linking group linking the η⁵-fluorenyl group (substituted or unsubstituted) and the η⁵-cyclopentadienyl group (substituted or unsubstituted) of the metallocene having the formula ZrR¹⁰R¹¹X⁹₂ may have the formula >CR¹R², >SiR³R⁴, or —CR⁵R⁶CR⁷R⁸—, and R¹, R², R³, R⁴, R⁵, R⁶, R⁷, and R⁸ independently are hydrogen or a C₁-C₂₀ hydrocarbyl group; alternatively, hydrogen or a C₁-C₁₀ hydrocarbyl group; alternatively, a hydrogen or a C₁-C₂₀ alkyl group; alternatively, hydrogen or a C₁-C₁₀ alkyl group; or alternatively, hydrogen or a C₁-C₅ alkyl group. In some particular embodiments, at least one of the R groups on the linking group is a C₃-C₁₂ alkenyl group. In an embodiment, the linking group linking the η⁵-fluorenyl group (substituted or unsubstituted) and the η⁵-cyclopentadienyl group (substituted or unsubstituted) of the metallocene having the formula ZrR¹⁰R¹¹X⁹₂ may have the formula >CR¹R² and R¹ and R² independently are hydrogen or a C₁-C₂₀ hydrocarbyl group; alternatively, hydrogen or a C₁-C₁₀ hydrocarbyl group; alternatively, a hydrogen or a C₁-C₂₀ alkyl group; alternatively, hydrogen or a C₁-C₁₀ alkyl group; alternatively, hydrogen or a C₁-C₅ alkyl group. In some particular embodiments, at least one of the R¹ or R² is a C₃-C₁₂ alkenyl group. In other embodiments, the linking group has the formula >CR¹R² wherein R¹ is a C₃-C₁₂ alkenyl group and R² is a hydrogen, C₁ to C₂₀ alkyl group or a C₆ to C₂₀ aryl group; R¹ is a C₃-C₁₂ alkenyl group and R² is a hydrogen, C₁ to C₁₀ alkyl group or a C₆ to C₁₀ aryl group; or alternatively, R¹ is a C₃-C₁₂ alkenyl group and R² is a C₁ to C₅ alkyl group or a C₆ to C₁₀ aryl group.

In another non-limiting embodiment, the metallocene of formula ZrR¹⁰R¹¹X⁹₂ can have the formula:

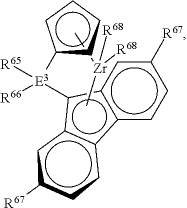

wherein E³ can be C, Si, Ge, or Sn, R⁶⁵ can be H or a C₁-C₂₀ hydrocarbyl group, R⁶⁶ can be a C₃-C₁₂ alkenyl group, R⁶⁷ can be H or a C₁-Cl₂ hydrocarbyl group, and R⁶⁸ can be H or a C₁-C₂₀ hydrocarbyl group.

In some particular embodiments, the metallocene of formula ZrR¹⁰R¹¹X⁹₂ may have the formula:

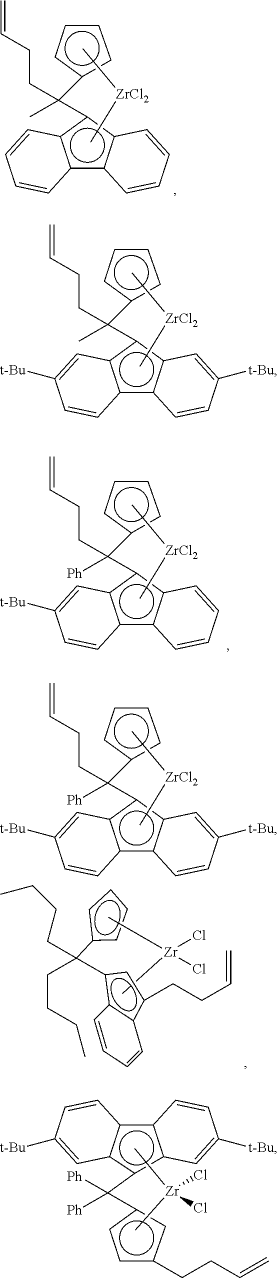

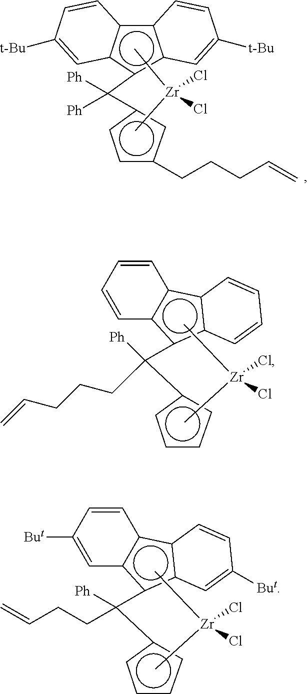

or any combination thereof; or alternatively, may have the formula

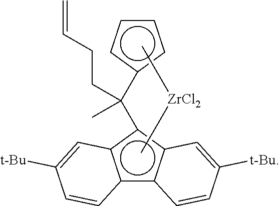

In yet another non-limiting embodiment, the metallocene may have the formula ZrR¹²R¹³R¹⁴X⁹₂ wherein each X⁹ independently is a halogen atom, R¹² is a neutral ether group, R¹³ is a η¹-aminyl group, R¹⁴ is a substituted or unsubstituted η¹-fluorenyl group, and wherein R¹³ and R¹⁴ are connected by a linking group. Within embodiments of the metallocene having the formula ZrR¹²R¹³R¹⁴X⁹₂, X⁹, R¹², R¹³, and R¹⁴ may be any halide disclosed herein, any neutral ether disclosed herein, any η¹-aminyl group disclosed herein, and any substituted or unsubstituted η¹-fluorenyl disclosed herein respectively. Additionally, the group linking R¹³ and R¹⁴ may be any linking group disclosed herein.

In an embodiment, X⁹ of the metallocene having the formula ZrR¹²R¹³R¹⁴X⁹₂ may be chlorine; or alternatively bromine. In an embodiment, X¹² of the metallocene having the formula ZrR¹²R¹³R¹⁴X⁹₂ may be any C₂-C₂₀ ether group disclosed herein. In an embodiment, the ether group may have the formula R¹⁵OR¹⁶ and R¹⁵ and R¹⁶ are independently selected from a C₁-C₂₀ hydrocarbyl group; alternatively, C₁-C₁₀ hydrocarbyl group; C₁-C₅ hydrocarbyl group; C₁-C₂₀ hydrocarbyl group; C₁-C₁₀ alkyl group; or alternatively C₁-C₅ alkyl group. In some embodiments, the ether group may be a C₁-C₁₀ cyclic ether; alternatively, a C₁-C₁₀ aliphatic cyclic ether. In other embodiments the ether may be dimethyl ether, diethyl ether, or dipropyl ether; alternatively, diethyl ethyl. In other embodiments, the ether group may be diphenyl ether or dibenzyl ether; alternatively, diphenyl ether; or alternatively, dibenzyl ether. In yet other embodiments, the ether group may be tetrahydrofuran, a substituted tetrahydrofuran, pyran, or a substituted pyran; alternatively, tetrahydrofuran or a substituted tetrahydrofuran; alternatively, pyran or a substituted pyran; alternatively, tetrahydrofuran.

In an embodiment, R¹³ of the metallocene having the formula ZrR¹²R¹³R¹⁴X⁹₂ may be any amidyl group disclosed herein. In an embodiment, the amidyl has the formula >NR¹⁷ wherein R¹⁷ is a C₁-C₂₀ hydrocarbyl group; a C₁-C₁₀ hydrocarbyl group; a C₁-C₁₀ alkyl group; or alternatively, C₁-C₅ alkyl group. Generally, the alkyl group may be any alkyl group disclosed herein.

In an embodiment, R¹⁴ of the metallocene having the formula ZrR¹²R¹³R¹⁴X⁹₂ may be any substituted η¹-fluorenyl group. Alternatively, R¹⁴ of the metallocene having the formula ZrR¹²R¹³R¹⁴X⁹₂ may be an unsubstituted η¹-fluorenyl group. Generally, the linking group linking the η¹-fluorenyl group (substituted or unsubstituted) and the amidyl group is attached at the 9 position of the η¹-fluorenyl group. In an embodiment, the substituted η¹-fluorenyl group has substituents located at the 2 and 7 positions; alternatively, has substituents located at the 2, 3, 6, and 7 positions; alternatively, excluding the linking group the η¹-fluorenyl group only has substituents located at 2 and 7 positions; alternatively, excluding the linking group the η¹-fluorenyl group only has substituent located at the 2, 3, 6, and 7 positions. In any embodiment, when the η¹-fluorenyl group has substituents and the 2, 3, 6, and 7 positions the group at the 2 and 3 positions may be joined to form a ring and/or the groups at the 6 and 7 positions may be joined to form a ring. In some particular embodiments, the substituents of the substituted η¹-fluorenyl group may be a C₁-C₂₀ hydrocarbyl group; alternatively, a C₁-C₁₀ hydrocarbyl group; alternatively, a C₁-C₂₀ alkyl group; a C₁-C₁₀ alkyl group; or alternatively, a C₁-C₅ alkyl group; or alternatively, a C₃-C₁₂ alkenyl group. If the 2 and 3 positions and/or the 6 and 7 positions are joined to form a ring the joined substituent group may be a C₁-C₂₀ hydrocarbylene group; alternatively, a C₁-C₁₀ hydrocarbylene group; alternatively, a C₁-C₂₀ alkylene group; a C₁-C₁₀ alkylene group; or alternatively, a C₁-C₅ alkylene group. In some embodiments, the substituted η¹-fluorenyl group is a substituted or unsubstituted (excluding the linking group) dibenzofluorene group or a substituted or unsubstituted (excluding the linking group) octahydrobenzofluorene group; alternatively a substituted or unsubstituted 2,3,6,7-dibenzofluorene group or a substituted or unsubstituted octahydro-2,3,6,7-benzofluorene group. In an embodiment, the linking group linking the η¹-fluorenyl group (substituted or unsubstituted) with the amidyl group of the metallocene having the formula ZrR¹²R¹³R¹⁴X⁹₂ may have the formula >CR¹R², >SiR³R⁴, or —CR⁵R⁶CR⁷R⁸—, and R¹, R², R³, R⁴, R⁵; R⁶, R⁷, and R⁸ are each selected independently from a hydrogen, and a C₁-C₂₀ hydrocarbyl group; alternatively, hydrogen or a C₁-C₁₀ hydrocarbyl group; alternatively, a hydrogen or a C₁-C₂₀ alkyl group; alternatively, hydrogen or a C₁-C₁₀ alkyl group; or alternatively, hydrogen or a C₁-C₅ alkyl group. In an embodiment, the linking group linking the η¹-fluorenyl group (substituted or unsubstituted) with the amidyl group of the metallocene having the formula ZrR¹²R¹³R¹⁴X⁹₂ may have the formula >CR¹R² and R¹ and R² independently are hydrogen or a C₁-C₂₀ hydrocarbyl group; alternatively, hydrogen or a C₁-C₁₀ hydrocarbyl group; alternatively, a hydrogen or a C₁-C₂₀ alkyl group; alternatively, hydrogen or a C₁-C₁₀ alkyl group; alternatively, hydrogen or a C₁-C₅ alkyl group; alternatively, C₁-C₂₀ hydrocarbyl groups; alternatively, C₁-C₁₀ hydrocarbyl groups; alternatively, C₁-C₂₀ alkyl groups; alternatively, C₁-C₁₀ alkyl groups; or alternatively, C₁-C₅ alkyl groups. In an embodiment, the linking group linking the η¹-fluorenyl group (substituted or unsubstituted) with the amidyl group of the metallocene having the formula ZrR¹²R¹³R¹⁴X⁹₂ may have the formula >SiR³R⁴ and R³ and R⁴ independently are hydrogen or a C₁-C₂₀ hydrocarbyl group; alternatively, hydrogen or a C₁-C₁₀ hydrocarbyl group; alternatively, a hydrogen or a C₁-C₂₀ alkyl group; alternatively, hydrogen or a C₁-C₁₀ alkyl group; alternatively, hydrogen or a C₁-C₅ alkyl group; alternatively, C₁-C₂₀ hydrocarbyl groups; alternatively, C₁-C₁₀ hydrocarbyl groups; alternatively, C₁-C₂₀ alkyl groups; alternatively, C₁-C₁₀ alkyl groups; or alternatively, C₁-C₅ alkyl groups.

In a non-limiting embodiment, the metallocene of formula ZrR¹²R¹³R¹⁴X⁹₂ may have the formula

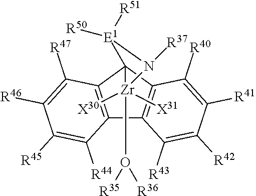

wherein, E¹ can be C, Si, Ge, or Sn; R⁴⁰, R⁴¹, R⁴², R⁴³, R⁴⁴, R⁴⁵, R⁴⁶, and R⁴⁷ independently can be hydrogen or a C₁ to C₂₀ hydrocarbyl group (saturated or unsaturated); R⁵⁰ and R⁵¹ independently can be a hydrogen, and saturated or unsaturated C₁-C₂₀ hydrocarbyl group; R³⁷ can be a C₁-C₂₀ hydrocarbyl group; and R³⁵OR³⁶ represents an ether group wherein R³⁵ and R³⁶ independently can be a C₁-C₂₀ hydrocarbyl group. In an embodiment, E¹ can be C or Si; alternatively, C; or alternatively Si. In an embodiment, R⁴⁰, R⁴¹, R⁴², R⁴³, R⁴⁴, R⁴⁵, R⁴⁶, and R⁴⁷ independently can be hydrogen or a C₁-C₁₀ hydrocarbyl group; alternatively, hydrogen or a C₁-C₂₀ alkyl group; alternatively, hydrogen, or a C₁-C₁₀ alkyl group; or alternatively, hydrogen or a C₁-C₅ alkyl group. In other embodiments, R⁴⁰, R⁴³, R⁴⁴, and R⁴⁷ can be hydrogen and R⁴¹, R⁴², R⁴⁵, and R⁴⁶ independently can be hydrogen or a C₁ to C₂₀ hydrocarbyl groups; alternatively, R⁴⁰, R⁴³, R⁴⁴, and R⁴⁷ can be hydrogen and R⁴¹, R⁴², R⁴⁵, and R⁴⁶ independently can be hydrogen or a C₁-C₁₀ hydrocarbyl group; alternatively, R⁴⁰, R⁴³, R⁴⁴, and R⁴⁷ can be hydrogen and R⁴¹, R⁴², R⁴⁵, and R⁴⁶ independently can be hydrogen or a C₁-C₂₀ alkyl group; alternatively, R⁴⁰, R⁴³, R⁴⁴, and R⁴⁷ can be hydrogen and R⁴¹, R⁴², R⁴⁵, and R⁴⁶ independently can be hydrogen or a C₁-C₁₀ alkyl group; or alternatively, R⁴⁰, R⁴³, R⁴⁴, and R⁴⁷ can be hydrogen and R⁴¹, R⁴⁵ and R⁴⁶, independently can be hydrogen or a C₁-C₅ alkyl group. In any embodiment wherein R⁴¹, R⁴², R⁴⁵, and R⁴⁶ are not hydrogen, R⁴¹ and R⁴² can be joined to form a ring and/or R⁴⁵ and R⁴⁶ can be joined to form a ring. In any embodiment where R⁴⁰ and R⁴² and/or are joined to form a ring, the joined group can be a C₁-C₂₀ hydrocarbylene group; alternatively, a C₁-C₁₀ hydrocarbylene group; alternatively, a C₁-C₂₀ alkylene group; a C₁-C₁₀ alkylene group; or alternatively, a C₁-C₅ alkylene group. In any embodiment, R³⁷ can be a C₁-C₁₀ hydrocarbyl group; a C₁-C₁₀ alkyl group; or alternatively, C₁-C₅ alkyl group. In any embodiment, R⁵⁰ and R⁵⁰ independently can be hydrogen or a C₁-C₂₀ hydrocarbyl group; alternatively, hydrogen or a C₁-C₁₀ hydrocarbyl group; alternatively, a hydrogen or a C₁-C₂₀ alkyl group; alternatively, hydrogen or a C₁-C₁₀ alkyl group; alternatively, hydrogen or a C₁-C₅ alkyl group; alternatively, C₁-C₂₀ hydrocarbyl groups; alternatively, C₁-C₁₀ hydrocarbyl groups; alternatively, C₁-C₂₀ alkyl groups; alternatively, C₁-C₁₀ alkyl groups; or alternatively, C₁-C₅ alkyl groups. Further, X³⁰ and X³¹ are as provided herein, and in some embodiments, X³⁰ and X³¹ independently are halogen atoms.

In a non-limiting embodiment, the metallocene of formula ZrR¹²R¹³R¹⁴X⁹₂ can have the formula

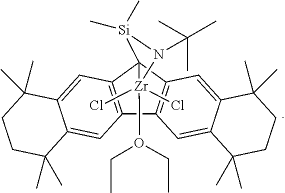

In a non-limiting embodiment, the alumoxane can be a) a cyclic alumoxane having the formula

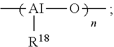

wherein R¹⁸ is a linear or branched alkyl having from 1 to 10 carbon atoms, and n is an integer from 3 to about 10, b) a linear aluminoxane having the formula

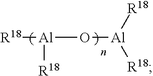

wherein R¹⁸ is a linear or branched alkyl having from 1 to 10 carbon atoms, and n is an integer from 1 to about 50, c) a cage aluminoxane having the formula R^{t}_{5m+α}R^{b}_{m-α}Al₄ₘO₃ₘ, wherein m is 3 or 4 and α is =n_{Al(3)}−n_{O(2)}+n_{O(4)}; wherein n_{Al(3)} is the number of three coordinate aluminum atoms, n_{O(2)} is the number of two coordinate oxygen atoms, n_{O(4)} is the number of 4 coordinate oxygen atoms, R^{t} represents a terminal alkyl group, and R^{b} represents a bridging alkyl group; wherein R is a linear or branched alkyl having from 1 to 10 carbon atoms; or d) any combination thereof. In an embodiment, the alumoxane can comprise a linear aluminoxane having the formula

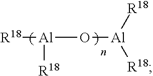

wherein R¹⁸ is a linear or branched alkyl having from 1 to 10 carbon atoms, and n is an integer from 1 to about 50. In some embodiments, the alumoxane can comprise methylaluminoxane, ethylaluminoxane, n-propylaluminoxane, iso-propylaluminoxane, n-butylaluminoxane, sec-butylaluminoxane, iso-butylaluminoxane, t-butyl aluminoxane, 1-pentylaluminoxane, 2-pentylaluminoxane, 3-pentylaluminoxane, iso-pentylaluminoxane, neopentylaluminoxane, or mixtures thereof. In other embodiments, the alumoxane can comprise methylaluminoxane (MAO), modified methylaluminoxane (MMAO), isobutyl aluminoxane, t-butyl aluminoxane, or mixtures thereof; or alternatively, a modified methylaluminoxane.

In a non-limiting embodiment, the chemically treated can be fluorided alumina, chlorided alumina, sulfated alumina, fluorided silica-alumina, or any combination thereof. In an embodiment, the chemically-treated solid oxide can be fluorided alumina; alternatively, chlorided alumina; alternatively, sulfated alumina; or alternatively, fluorided silica-alumina.

In a non-limiting embodiment, the organoaluminum compound can have the formula Al(X¹⁰)ₙ(X¹¹)₃₋ₙ wherein each X¹⁰ can be independently a C₁ to C₂₀ hydrocarbyl group, each X¹¹ independently can be a halide, a hydride, or a C₁ to C₂₀ hydrocarboxide group, and n can be a number from 1 to 3. In other embodiments, each X¹⁰ can be independently a C₁ to C₁₀ hydrocarbyl group; alternatively, a C₆ to C₂₀ aryl group; alternatively, a C₆ to C₁₀ aryl group; alternatively, a C₁ to C₂₀ alkyl group; alternatively, a C₁ to C₁₀ alkyl group; or alternatively, a C₁ to C₅ alkyl group. In other embodiments, each X¹¹ can be independently a halide, a hydride, or a C₁ to C₂₀ hydrocarboxide group; alternatively, a halide, a hydride, or a C₁ to C₁₀ hydrocarboxide group; alternatively, a halide, a hydride, or a C₆ to C₂₀ aryloxide group; alternatively, a halide, a hydride, or a C₀ to C₁₀ aryloxide group; alternatively, a halide, a hydride, or a C₁ to C₂₀ alkoxide group; alternatively, a halide, a hydride, or a C₁ to C₁₀ alkoxide group; or alternatively, a halide, a hydride, or a C₁ to C₅ alkoxide group. In an embodiment, and n can be a number from 1 to 3; alternatively 1; alternatively, 1.5; alternatively, 2; or alternatively, 3. In an embodiment, the organoaluminum compound can comprise a trialkylalumium compound, a dialkylaluminum halide compound, an alkylaluminum dihalide, or a combination thereof; alternatively, a trialkylalumium compound, a dialkylaluminum halide compound, or a combination thereof. In some embodiments, the organoaluminum compound can comprise, or consist essentially of, trimethylaluminum, triethylaluminum, ethylaluminum sesquichloride, tripropylaluminum, tributylaluminum, diethylaluminum ethoxide, tri-n-butylaluminum, disobutylaluminum hydride, triisobutylaluminum, diethylaluminum chloride, or any combination thereof. In other embodiments, the organoaluminum compound can comprise, or consist essentially of, a trialkyl aluminum compound. In yet other embodiments, the organoaluminum compound can comprise, or consist essentially of, trimethylaluminum, triethylaluminum, tripropylaluminum, tri-n-butylaluminum, triisobutylaluminum, tri-n-hexylaluminum, trioctylaluminum, or any combination thereof; alternatively, triethylaluminum; alternatively, tripropylaluminum; alternatively, tri-n-butylaluminum; alternatively, triisobutylaluminum; alternatively, tri-n-hexylaluminum; or alternatively, trioctylaluminum.

In an embodiment when the catalyst system comprises a metallocene and an alumoxane, the molar ratio of the metal of the alumoxane to metal of the metallocene can be at least 1:1; alternatively, 100:1; alternatively, 250:1; or alternatively, 500:1. Alternatively, the molar ratio of the metal of the alumoxane to metal of the metallocene can range from 1:1 to 100,000:1; alternatively, 100:1 to 10,000:1; alternatively, 250:1 to 7,500:1; or alternatively, 500:1 to 5,000:1.

In an embodiment when the catalyst system comprises a metallocene and chemically treated solid oxide or a metallocene, a chemically-treated solid oxide, and an organoaluminum compound, the weight ratio of the chemically-treated solid oxide to metallocene can be at least 1:1; alternatively, at least 5:1; alternatively, at least 10:1; or alternatively, at least 25:1. Alternatively, the weight ratio of the chemically-treated solid oxide to metallocene can range from 1:1 to 10,000; alternatively, 5:1 to 5,000:1; alternatively, 10:1 to 1,000:1; or alternatively, 25:1 to 750:1.

In an embodiment, wherein the catalyst system comprises a metallocene, a chemically-treated solid oxide, an organoaluminum compound, the molar ratio of the metal of the organoaluminum compound to the metal of the metallocene can be at least 0.1:1; alternatively, at least 1:1; alternatively, at least 5:1; or alternatively, at least 10:1. Alternatively, the molar ratio of the metal of the organoaluminum compound to the metal of the metallocene can range from 0.1:1 to 10,000; alternatively, 1:1 to 5,000:1; alternatively, 5:1 to 2,500:1; or alternatively, 25:1 to 1,500:1.

Further, in one aspect, activators such as aluminoxanes, organoboron compounds, ionizing ionic compounds, organozinc compounds, or any combination thereof can be used as activators with the metallocene, either in the presence or in the absence of the chemically treated solid oxide, and either in the presence or in the absence of the organoaluminum compounds.

### Method for Producing an Olefin Wax Oligomer or Oligomerizing an Olefin Wax

In an aspect, this disclosure encompasses a method of producing an olefin wax oligomer and/or olefin wax oligomer composition, a method of oligomerizing an olefin wax. In an embodiment, a method disclosed herein can be a method of producing olefin wax oligomer and/or an olefin wax oligomer composition, the method comprising: a) contacting an olefin wax and a catalyst system, and b) oligomerizing the olefin wax under oligomerization conditions. In an embodiment, a method disclosed herein can be a method of oligomerizing an olefin wax, the method comprising: a) contacting an olefin wax and a catalyst system, and b) oligomerizing the olefin wax under oligomerization conditions. In an embodiment, a method disclosed herein can be a method to produce any olefin wax oligomer and/or olefin wax oligomer composition described herein, the method comprising: a) contacting an olefin wax and a catalyst system, and b) oligomerizing the olefin wax under oligomerization conditions. Catalyst systems which can be utilized within these methods are disclosed herein and can be utilized without limitation to further describe the methods. Additionally, the methods can contain other steps such as deactivating the catalyst system, removing the catalyst system, and/or removing deactivated catalyst system components from the olefin wax oligomer composition, among other method steps. The other method steps can be utilized without limitation to further describe the methods.

The olefin waxes which can be utilized in the methods may be any olefin wax described herein. In an exemplary, but non-limiting, embodiment, the olefin wax can be an alpha olefin wax; or alternatively, a normal alpha olefin wax. In one exemplary, but non-limiting, embodiment, the olefin wax can be an olefin wax having 70 wt % olefins having from 20 to 24 carbon atoms, an olefin wax having 60 wt % olefins having from 24 to 28 carbon atoms, an olefin wax having 70 wt % olefins having from 26 to 28 carbon atoms, or an olefin wax having 70 wt % olefins having greater than 30 carbon atoms. In another exemplary, but non-limiting embodiment, the olefin wax can be an olefin wax having 70 wt % olefins having from 20 to 24 carbon atoms; alternatively, an olefin wax having 60 wt % olefins having from 24 to 28 carbon atoms; alternatively, an olefin wax having 70 wt % olefins having from 26 to 28 carbon atoms; or alternatively, an olefin wax having 70 wt % olefins having greater than 30 carbon atoms. In a further exemplary, but non-limiting, embodiment, the olefin wax can be an olefin wax having 70 wt % olefins having from 20 to 24 carbon atoms and greater than 70 mole % alpha olefin, an olefin wax having 60 wt % olefins having from 24 to 28 carbon atoms and greater than 45 mole % alpha olefin, an olefin wax having 70 wt % olefins having from 26 to 28 carbon atoms and greater than 75 mole % alpha olefin, or an olefin wax having 70 wt % olefins having greater than 30 carbon atoms and greater than 45 mole % alpha olefin. In yet another exemplary, but non-limiting, embodiment, the olefin wax can be an olefin wax having 70 wt % olefins having from 20 to 24 carbon atoms and greater than 70 mole % alpha olefin; alternatively, an olefin wax having 60 wt % olefins having, from 24 to 28 carbon atoms and greater than 45 mole % alpha olefin; alternatively, an olefin wax having 70 wt % olefins having from 26 to 28 carbon atoms and greater than 75 mole % alpha olefin; or alternatively, an olefin wax having 70 wt % olefins having greater than 30 carbon atoms and greater than 45 mole % alpha olefin. Other olefin waxes and olefin wax features are disclosed herein and may be utilized, without limitation, to describe the olefin wax that may be utilized in the methods described herein.

The olefin wax oligomerizations according to this disclosure can be carried out in any manner known in the art suitable for the specific olefin waxes employed in the oligomerization process. For example, the oligomerization processes can include, but are not limited to batch process. Alternatively the oligomerization can be carried out continuously in a loop reactor or in a continuous stirred reactor.

In an embodiment, the weight ratio of the olefin wax to the metallocene can be greater than 100:1; alternatively, greater than 1,000:1; alternatively, greater than 5,000:1; or alternatively, greater than 10,000:1. Alternatively, the weight ratio of the olefin wax to the metallocene can range from 100:1 to 10,000,000:1; alternatively, 1,000:1 to 1,000,000:1; alternatively 5,000:1 to 500,000:1; or alternatively, 10,000:1 to 1000,000:1.

In any embodiment, disclosed herein the oligomerization can be performed in the presence of a solvent. In other embodiments, the oligomerization can be performed in the absence of a solvent.

Generally, the olefin wax can be oligomerized at a temperature greater than the melting point of the wax; or alternatively, when a solvent is utilized the olefin wax can be oligomerized at a temperature sufficient to ensure that the olefin wax is completely dissolved in the solvent. In an embodiment, the oligomerization can be performed at a temperature greater than 40° C.; alternatively, greater than 50° C.; alternatively, greater than 60° C.; or alternatively, greater than 70° C. In some embodiments, the olefin wax can be oligomerized at a temperature ranging from the melting point of the olefin wax to 200° C.; or alternatively, when a solvent is utilized, the olefin wax can be oligomerized at a temperature ranging from a temperature sufficient to ensure that the olefin wax is completely dissolved in the solvent and 200° C. In other embodiments, the oligomerization can performed at a temperature ranging from 40° C. to 150° C.; alternatively, ranging from 50° C. to 130° C.; alternatively, ranging from 60° C. to 110° C.; or alternatively, ranging from 70° C. to 100° C.

The oligomerization reaction can be performed in an inert atmosphere, that is, in atmosphere substantially free of oxygen (e.g. less than 100, 50, 10, 5 or 1 ppm of oxygen) and under substantially anhydrous conditions, thus, in the substantial absence of water (e.g. less than 100, 50, 10, 5 or 1 ppm of water) as the reaction begins. Therefore a dry, inert atmosphere, for example, dry nitrogen, or dry argon, can be employed in the oligomerization reactor.

Another aspect and in any embodiment disclosed herein, the oligomerizations can be carried out in the presence of hydrogen or in the substantial absence of hydrogen (e.g. a partial pressure of hydrogen of less than 10 psig; alternatively, less than 7 psig; alternatively, less than 5 psig; alternatively, less than 4 psig; alternatively, less than 3 psig; alternatively, less than 2 psig; or alternatively, less than 1 psig of ethylene pressure).

In an aspect, the olefin oligomerization may be carried out in the presence of hydrogen. Generally, and while not intending to be bound by theory, hydrogen can be used in the oligomerization process to control oligomer molecular weight. In any embodiment or aspect disclosed here, the olefin wax oligomerization can be conducted with a partial pressure of hydrogen greater than or equal to 10 psig; alternatively, greater than or equal to 15 psig; alternatively, alternatively, greater than or equal to 20 psig; alternatively, or alternatively, greater than or equal to 25 psig. In other embodiments, the olefin wax oligomerization can be conducted with a partial pressure of hydrogen ranging from 10 psig to 5,000 psig; alternatively, ranging from 15 psig to 1,000 psig; alternatively, ranging from 20 psig to 750 psig; or alternatively, ranging from 25 psig to 500 psig.

The olefin wax oligomerization described herein can be carried out in the absence of an organic solvent. In an embodiment, the olefin wax oligomerization can be carried out in the presence of an organic solvent. Illustrative organic solvent types which can be utilized for the olefin wax oligomerization can include, but are not limited to, aliphatic hydrocarbons, aromatic hydrocarbons, halogenated aliphatic hydrocarbons, halogenated aromatic hydrocarbons, and combinations thereof; alternatively, aliphatic hydrocarbons; alternatively, aromatic hydrocarbons; alternatively, halogenated aliphatic hydrocarbons; or alternatively, halogenated aromatic hydrocarbons. Aliphatic hydrocarbons which can be useful as an organic solvent include C₃ to C₂₀ aliphatic hydrocarbons; alternatively, C₃ to C₁₅ aliphatic hydrocarbons; or alternatively, C₄ to C₁₀ aliphatic hydrocarbons. The aliphatic hydrocarbons may be cyclic or acyclic and/or may be linear or branched, unless otherwise specified. Non-limiting examples of suitable acyclic aliphatic hydrocarbon solvents that may be utilized singly or in any combination include propane, iso-butane, butane, pentane (n-pentane or mixture of linear and branched C₅ acyclic aliphatic hydrocarbons), hexane (n-hexane or mixture of linear and branched C₆ acyclic aliphatic hydrocarbons), heptane (n-heptane or mixture of linear and branched C₇ acyclic aliphatic hydrocarbons), octane, and combinations thereof. Non-limiting examples of suitable cyclic aliphatic hydrocarbon solvents include cyclohexane, methyl cyclohexane. Aromatic hydrocarbons which can be useful as an organic solvent include C₆ to C₂₀ aromatic hydrocarbons; or alternatively, C₆ to C₁₀ aromatic hydrocarbons. Non-limiting examples of suitable aromatic hydrocarbons that can be utilized singly or in any combination include benzene, toluene, xylene (including ortho-xylene, meta-xylene, para-xylene, or mixtures thereof), and ethylbenzene, or combinations thereof. Halogenated aliphatic hydrocarbons which can be useful as an organic solvent include C₁ to C₁₅ halogenated aliphatic hydrocarbons; alternatively, C₁ to C₁₀ halogenated aliphatic hydrocarbons; or alternatively, C₁ to C₅ halogenated aliphatic hydrocarbons. The halogenated aliphatic hydrocarbons can be cyclic or acyclic and/or can be linear or branched, unless otherwise specified. Non-limiting examples of suitable halogenated aliphatic hydrocarbons which can be utilized singly of in any combination include methylene chloride, chloroform, carbon tetrachloride, dichloroethane, trichloroethane, and combinations thereof. Halogenated aromatic hydrocarbons which can be useful as an organic solvent include C₆ to C₂₀ halogenated aromatic hydrocarbons; or alternatively, C₆ to C₁₀ halogenated aromatic hydrocarbons. Non-limiting examples of suitable halogenated aromatic hydrocarbons that can be utilized singly or in any combination include chlorobenzene, dichlorobenzene, and combinations thereof.

In an aspect of this disclosure, the methods utilizing a catalyst system comprising, or consisting essentially of a metallocene, a chemically-treated solid oxide, and an organoaluminum compound disclosed herein can optionally include a step of precontacting the metallocene with the olefin wax monomer to be oligomerized, and an organoaluminum compound for a first period of time prior to contacting this precontacted mixture with the chemically treated solid oxide. In one aspect, the first period of time for contact, the precontact time, between the metallocene compound or compounds, the olefin wax monomer, and the organoaluminum compound can range from 0.1 hour to 24 hours, and from 0.1 to 1 hour. Precontact times from 10 minutes to 30 minutes can also be utilized. In an embodiment, once the precontacted mixture of metallocene compound, olefin monomer, and organoaluminum compound is contacted with the chemically treated solid oxide, this composition (further comprising the chemically treated solid oxide) can termed the postcontacted mixture. Typically, the postcontacted mixture can optionally be allowed to remain in contact for a second period of time, the postcontact time, prior to being initiating the oligomerization process. In one aspect, postcontact times between the precontacted mixture and the chemically treated solid oxide can range in time from 0.1 hour to 24 hours. In another aspect, for example, postcontact range can range from 0.1 hour to 1 hour. In one aspect, the precontacting, the postcontacting step, or both may increase the productivity of the oligomerization as compared to the same catalyst system that is prepared without precontacting or postcontacting. However, neither a precontacting step nor a postcontacting step is required for a particular method.

In an aspect of this disclosure, the methods utilizing a catalyst system comprising a metallocene, a chemically-treated solid oxide, and an organoaluminum, the postcontacted mixture can be maintained at a temperature and for a duration sufficient to allow adsorption, impregnation, or interaction of precontacted mixture and the chemically treated solid oxide, such that a portion of the components of the precontacted mixture can be immobilized, adsorbed, or deposited thereon. For example, the postcontacted mixture can be maintained at a temperature ranging from 0° F. to 150° F.; alternatively from 40° F. to 95° F.

For purposes of the disclosure, the term oligomerization reactor includes any oligomerization reactor or oligomerization reactor system known in the art that is capable of oligomerizing the particular olefin wax monomers to produce olefin wax oligomers according to the present disclosure. Oligomerization reactors suitable for the present disclosure can comprise at least one raw material feed system, at least one feed system for the catalyst system or catalyst system components, at least one reactor system, at least one oligomer recovery system or any suitable combination thereof. Suitable reactors for the present disclosure can further comprise any one, or combination of, a catalyst system storage system, catalyst system component storage system, a cooling system, a diluent or solvent recycling system, a monomer recycling system, and/or a control system. Such reactors can comprise continuous take-off and direct recycling of catalyst, diluent, monomer, and oligomer.

Oligomerization reactor systems of the present disclosure can comprise one type of reactor per system or multiple reactor systems comprising two or more types of reactors operated in parallel or in series. Multiple reactor systems can comprise reactors connected together to perform oligomerization, or reactors that are not connected. The olefin wax monomer can be oligomerized in one reactor under one set of conditions, and then the olefin wax oligomers can be transferred to a second reactor for oligomerization under a different set of conditions.

In one aspect of the disclosure, the oligomerization reactor system can comprise a batch reactor. The oligomerization can be performed using the olefin wax monomer in the presence or absence of an organic solvent. Exemplary solvents are disclosed herein and can be utilized without limitation to disperse and/or carry the catalyst system. Olefin wax monomer, solvent, catalyst system components, and/or catalyst system, can be separately fed to the batch reactor where oligomerization occurs. Alternatively, the catalyst system and/or one or more of the catalyst system components can be dispersed and/or carried in the olefin wax monomer and then fed to the batch reactor.

In another aspect of the disclosure, the oligomerization reactor system can comprise at least one loop reactor. Such reactors are known in the art and can comprise vertical or horizontal loops. Such loops can comprise a single loop or a series of loops. Multiple loop reactors can comprise both vertical and horizontal loops. The oligomerization can be performed using the olefin wax monomer as the liquid carrier to disperse and/or carry the catalyst system components and/or catalyst system to the reactor; alternatively, an organic solvent can be used to disperse and/or carry the catalyst system components and/or catalyst system to the reactor. An organic solvent can also be utilized to reduce the viscosity of the reaction mixture (including the alpha olefin oligomers) and allow the reaction mixture to easily flow or be pumped through the process equipment. Exemplary organic solvents are disclosed herein and can be utilized without limitation to disperse and/or carry the catalyst system. Olefin wax monomer, solvent, catalyst system components and/or catalyst system, can be continuously fed to a loop reactor where oligomerization occurs.

In still another aspect of the disclosure, the oligomerization reactor can comprise a tubular reactor. Tubular reactors can be utilized to make oligomers by free radical initiation, or by employing the catalysts typically used for coordination oligomerization. Tubular reactors can have several zones where fresh monomer, catalyst system components, and/or catalyst system can be added.

In a further aspect of the disclosure, the oligomerization reactor system can comprise the combination of two or more reactors. Production of oligomers in multiple reactors can include several stages in at least two separate oligomerization reactors interconnected by a transfer device making it possible to transfer the olefin wax oligomers resulting from the first oligomerization reactor into the second reactor. The desired oligomerization conditions in one of the reactors can be different from the operating conditions of the other reactors. Alternatively, oligomerization in multiple reactors can include the manual transfer of olefin wax oligomer from one reactor to subsequent reactors for continued oligomerization.

In an aspect, the process can include a step to deactivate the catalyst system and/or remove the catalyst system and/or catalyst components from the olefin wax oligomer. In an embodiment, the catalyst system can be deactivated by contacting the product of the olefin wax oligomerization with water, an alcohol, a ketone, or any combination thereof. Alternatively, the catalyst system can be deactivated by contacting the product of the olefin wax oligomerization with a mixture of an alcohol and water; alternatively a mixture of an alcohol ketone; alternatively, an alcohol; alternatively, a ketone; or alternatively, water. The alcohol which can be utilized in any embodiment utilizing an alcohol for deactivating the catalyst system can comprise, or consist essentially of, a C₁ to C₁₀ alcohol; or alternatively, a C₁ to C₅ alcohol. In an embodiment, the alcohol which can be utilized in any embodiment utilizing an alcohol for deactivating the catalyst system can comprise, or consist essentially of, methanol, ethanol, isopropanol, or any combination thereof; alternatively, methanol; alternatively, ethanol; or alternatively, isopropanol. The ketone which can be utilized in any embodiment utilizing a ketone for deactivating the catalyst system can comprise, or consist essentially of, a C₃ to C₁₀ alcohol. In an embodiment, the ketone which can be utilized in any embodiment utilizing a ketone for deactivating the catalyst system can comprise, or consist essentially of, acetone.

In an aspect, the process can include a step to separate catalyst system, the catalyst system, the deactivated catalyst system, or deactivated catalyst system components from the product of the olefin wax oligomerization. In an embodiment, the separation step can include contacting the product of the olefin wax oligomerization with and wash solvent. Generally, the product of the olefin wax oligomerization and the wash solvent can be contacted at temperature and concentration at which the product of the olefin wax oligomerization is substantially dissolved in the solvent. The solution can then be filtered to remove the insoluble solids (catalyst system, the catalyst system, the deactivated catalyst system, or deactivated catalyst system components). Organic solvents have been described as solvent for the olefin wax oligomerization. These solvent can be utilized without limitation as the wash solvent for the separation step.

All publications and patents mentioned in this disclosure are incorporated herein by reference in their entireties, for the purpose of describing and disclosing the constructs and methodologies described in those publications, which might be used in connection with the methods of this disclosure. Any publications and patents discussed above and throughout the text are provided solely for their disclosure prior to the filing date of the present application. Nothing herein is to be construed as an admission that the inventors are not entitled to antedate such disclosure by virtue of prior invention.

Unless indicated otherwise, when a range of any type is disclosed or claimed, for example a range of the number of carbon atoms, viscosities, viscosity indices, pour points, Bernoulli indices, temperatures, and the like, it is intended to disclose or claim individually each possible number that such a range could reasonably encompass, including any sub-ranges encompassed therein. For example, when describing a range of the number of carbon atoms, each possible individual integral number and ranges between integral numbers of atoms that the range includes are encompassed therein. Thus, by disclosing a C₁ to C₁₀ alkyl group or an alkyl group having from 1 to 10 carbon atoms or “up to” 10 carbon atoms, Applicants' intent is to recite that the alkyl group can have 1, 2, 3, 4, 5, 6, 7, 8, 9, or 10 carbon atoms, and these methods of describing such a group are interchangeable. When describing a range of measurements such as a range of oligomerization temperatures, every possible number that such a range could reasonably encompass can, for example, refer to values within the range with one significant digit more than is present in the end points of a range. In this example, a temperature between 70° C. and 85° C. includes individually temperatures of 70° C., 71° C., 72° C., 73° C., 74° C., 75° C., 76° C., 77° C., 78° C., 79° C., 80° C., 81° C., 82° C., 83° C., 84° C., and 85° C. Applicants' intent is that these two methods of describing the range are interchangeable. Moreover, when a range of values is disclosed or claimed, which Applicants intent to reflect individually each possible number that such a range could reasonably encompass, Applicants also intend for the disclosure of a range to reflect, and be interchangeable with, disclosing any and all sub-ranges and combinations of sub-ranges encompassed therein. In this aspect, Applicants' disclosure of a C₁ to C₁₀ alkyl group is intended to literally encompass a C₁ to C₆ alkyl, a C₄ to C₈ alkyl, a C₂ to C₇ alkyl, a combination of a C₁ to C₃ and a C₅ to C₇ alkyl, and so forth. Accordingly, Applicants reserve the right to proviso out or exclude any individual members of any such group, including any sub-ranges or combinations of sub-ranges within the group, if for any reason Applicants choose to claim less than the full measure of the disclosure, for example, to account for a reference that Applicants are unaware of at the time of the filing of the application.

In any application before the United States Patent and Trademark Office, the Abstract of this application is provided for the purpose of satisfying the requirements of 37 C.F.R. §1.72 and the purpose stated in 37 C.F.R. §1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that may be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

For any particular compound disclosed herein, the general structure presented is also intended to encompass all conformational isomers and stereoisomers that may arise from a particular set of substituents, unless indicated otherwise. Thus, the general structure encompasses all enantiomers, diastereomers, and other optical isomers whether in enantiomeric or racemic forms, as well as mixtures of stereoisomers, as the context permits or requires. For any particular formula that is presented, any general formula presented also encompasses all conformational isomers, regioisomers, and stereoisomers that may arise from a particular set of substituents.

The present disclosure is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims.

In the following examples, unless otherwise specified, the syntheses and preparations described therein were carried out under an inert atmosphere such as nitrogen and/or argon. Solvents were purchased from commercial sources and were typically dried prior to use. Unless otherwise specified, reagents were obtained from commercial sources.

### EXAMPLES

### General Procedures and Reagents

The olefin waxes used in the Examples are Chevron Phillips Chemical Company (“CPChem”) ALPHAPLUS® normal alpha olefin (NAO) waxes, having the designation ALPHAPLUS® C20-24 (also designated C_{20/24} or C₂₀₋₂₄), ALPHAPLUS® C24-28 (C_{24/28} or C₂₄₋₂₈), ALPHAPLUS® C26-28 (C_{26/28} or C₂₆₋₂₈), ALPHAPLUS® C30+HA (C_{30+HA}), and ALPHAPLUS® C30+(C₃₀₊), where the carbon count represents the highest proportion of olefins in the product. Reference to equivalents is molar equivalents throughout. Reactions performed under an inert atmosphere were generally performed under, but are not limited to, dry nitrogen. Examples 1-3 and used Metallocene I.

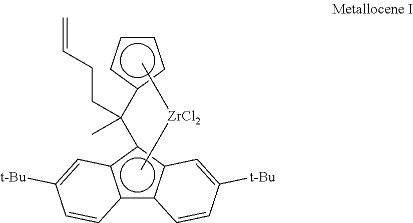

Molecular weights (MW) and molecular weight distributions were determined by Gel Permeation Chromatography (GPC), in which the GPC samples were measured in trichlorobenzene at 150° C. using SEC-FTIR. The Mark-Houwink-Sakurada constants associated for polyethylene in trichlorobenzene were used. The GPC numbers are expected to be slightly lower than the actual values, according to the literature (see Sun et al., Macromolecules 2001, 34, 6812).

Characterization of the olefin wax oligomers included the following tests. The drop melt point was measured according to ASTM D 127 and reported in ° F. The oil content was determied by MEK (methyl ethyl ketone) extraction, and reported in weight percent Measurements of hardness were carried out by needle penetration tests, determined at 77° F., 100° F., and @ 110° F. and reported in dmm (decimillimeters), according to ASTM D1321. This test measures the distance that a weighted needle or cone will sink into a sample during a set period of time at a prescribed temperature. Penetration results are presented in units of 0.1 mm (that is, the units are given as decimillimeters, dmm); therefore, a penetration of 40 means the needle has penetrated 4 mm. Flash point was determined according to ASTM D 93, and reported in ° F. and ° C. Saybolt Chromometer Method according to ASTM D156-07a was used to determine the Saybolt color of the olefin wax oligomers, which is reported in Saybolt color units. Kinematic viscosity was determined according to ASTM D445 at a temperature of 100° C., the results being reported in centistokes (cSt).

Preparation of a Fluorided Silica-Alumina Activator-Support (fSSA or FSSA)

The silica-alumina used to prepare the fluorided silica-alumina acidic activator-support in this Example was obtained from W.R. Grace as commercial Grade MS13-110, containing 13 weight % alumina, and having a pore volume of about 1.2 cc/g and a surface area of about 400 m²/g. This material was fluorided by impregnation to incipient wetness with a solution containing ammonium bifluoride, in an amount sufficient to equal 10 wt % of the weight of the silica-alumina. This impregnated material was then dried in a vacuum oven for 8 hours at 100° C. The thus-fluorided silica-alumina samples were then calcined as follows. About 10 grams of the fluorided silica-alumina were placed in a 1.75-inch quartz tube fitted with a sintered quartz disk at the bottom. While the fluorided silica-alumina was supported on the disk, dry air was blown up through the disk at the linear rate of about 1.6 to 1.8 standard cubic feet per hour. An electric furnace around the quartz tube was used to increase the temperature of the tube at the rate of about 400° C. per hour to a final temperature of about 450° C. At this temperature, the silica-alumina was allowed to fluidize for three hours in the dry air. Afterward, the silica-alumina was collected and stored under dry nitrogen, and was used without exposure to the atmosphere.

### Example 1

Under air- and moisture-free conditions, 50 g of CPChem C_{20/24} normal alpha olefin wax, as described and disclosed in this application, was heated to 75° C. with stirring, and purged with nitrogen for several hours. To the heated and purged wax was added 1000 molar equivalents (Al:Zr) of MMAO-3A (Akzo Nobel) followed immediately by 2.6 mg of Metallocene 1 dissolved 2.6 mL of anhydrous toluene. The reaction was maintained at 75° C., with stirring and under an inert atmosphere (dry nitrogen) for 3 days. After this time the reaction solution was poured warm (in the air) into a mixture of excess methanol and acetone sufficient to precipitate all the solids from the mixture. The solution was then decanted to remove the liquids. The solids were then washed thoroughly with heptane and pentane. During the washing procedure, the solids were crushed using a pestle to help dissolve any heptane and pentane soluble materials. After washing, 41 g of solids were isolated (83% isolated yield). Comparison of a 0.4% solution of the starting material and a 0.4% solution of the product in xylene using gas chromatography indicated that isolated solid sample contained about 34 g of olefin wax oligomers (68% polymer yield). Note that the GC analysis did not show olefin oligomer peaks; percent olefin wax monomer and olefin wax oligomers in the sample were determined by comparison of the response of the olefin wax monomer in the two samples. The solid sample was then subjected to GP (as described herein). The molecular weight information for the solid sample is reported in Table 1.

### Example 2

Under air- and moisture-free conditions, 60 g of CPChem C_{26/28} normal alpha olefin wax was heated to 75° C. with stirring, and purged with nitrogen for several hours. To the heated and purged wax was added 1000 equivalents (Al:Zr) of MMAO-3A followed immediately by 2.6 mg of Metallocene I dissolved 2.6 mL of toluene. The reaction was maintained at 75° C., with stirring and under inert atmosphere, for 18 hours. The reaction solution was then poured, hot, into heptane. The solution was then decanted to remove the liquids. The solids were then washed thoroughly with heptane, which solubilizes the starting wax, followed by pentane to remove the heptane. During the washing procedure, the solids were crushed using a pestle to help dissolve any heptane and pentane soluble materials. After washing, 34 g of solids were isolated (83% isolated yield). Comparison of a 0.4% solution of the starting material and a 0.4% solution of the product in xylene using gas chromatography indicated that isolated solid sample contained about 72% olefin wax oligomers and 28% olefin wax monomer. Note that the GC analysis did not show olefin oligomer peaks; percent olefin wax monomer and olefin wax oligomers in the sample were determined by comparison of the response of the olefin wax monomer in the two samples. The GC analysis thus indicated that the oligomerization produced about 24.5 g of olefin wax oligomers (41% polymer yield). The solid sample was then subjected to GP (as described herein). The molecular weight information for the solid sample is reported in Table 1.

### Example 3

Under air- and moisture-free conditions, 60 g of CPChem C_{30+HA} normal alpha olefin wax was heated to 75° C. with stirring, and purged with nitrogen for several hours. To the heated and purged wax was added 1000 equivalents (Al:Zr) of MMAO-3A followed immediately by 2.6 mg of Metallocene I dissolved 2.6 mL of toluene. The reaction was maintained at 75° C., with stirring and under inert atmosphere, for 18 hours. The reaction solution was then poured, hot, into heptane. The solution was then decanted to remove the liquids. The solids were then washed thoroughly with heptane and pentane. During the washing procedure, the solids were crushed using a pestle to help dissolve any heptane and pentane soluble materials. After washing, 46 g of solids were isolated (77% isolated yield). Comparison of a 0.4% solution of the starting material and a 0.4% solution of the product in xylene using gas chromatography indicated that isolated solid sample contained about 56% olefin wax oligomers and 44% olefin wax monomer. Note that the GC analysis did not show olefin oligomer peaks; percent olefin wax monomer and olefin wax oligomers in the sample were determined by comparison of the response of the olefin wax monomer in the two samples. The GC analysis thus indicated that the oligomerization produced about 25.8 g of olefin wax oligomers (43% polymer yield). The solid sample was then subjected to GPC as described herein. The molecular weight information for the solid sample is reported in Table 1, with comparative data for VyBar® 260 illustrated.

TABLE 1
Molecular Weight (MW) Properties of Oligomerized Olefin Waxes

| | Peak Oligomer | Mₙ | M_{w} | Polydispersity |
|---|---|---|---|---|
| | MW (amu) | (amu) | (amu) | Index |
| Example 1 | 9,949 | 2,787 | 11,986 | 4.3 |
| Example 2 | 13221 | 1,400 | 14,230 | 10.16 |
| Example 3 | 11,502 | 1,144 | 9,966 | 8.71 |
| VyBar ® 260 | 3,477 | 2,127 | 20,077 | 9.44 |

### Example 4

Under air- and moisture-free conditions, 20 g of CPChem C_{20/24} normal alpha olefin wax was heated to 80° C. with stirring under a nitrogen atmosphere. To the heated wax was added, with stirring, 120 mg trisobutylaluminum in 0.6 mL of toluene followed immediately by 0.96 mg of bis-indenyl zirconium dichloride dissolved 0.48 mL of toluene. To this solution was added, with stirring, 200 mg of fluorided silica-alumina (fSSA or FSSA) prepared as provided herein. The reaction was maintained at 80° C., with stirring, for 8 hours. The reaction solution was quenched by adding 0.5 mL of water while the reaction solution was hot. The product was then poured into heptane and the insoluble solids were removed by filtration. The solid material was then subjected to vacuum to remove heptane. The product obtained was a white waxy material.

### Example 5

Under air- and moisture-free conditions, 20 g of CPChem C_{30+HA} normal alpha olefin wax was heated to 80° C. with stirring under a nitrogen atmosphere. To the heated wax was added, with stirring, 120 mg trisobutylaluminum in 0.6 mL of toluene followed immediately by 0.96 mg of bis-indenyl zirconium dichloride dissolved 0.48 mL of toluene. To this solution was added, with stirring, 200 mg of FSSA. The reaction was maintained at 80° C., with stirring, for 8 hours. The reaction solution was quenched by adding 0.5 mL of water while the reaction solution was hot. The product was then poured into heptane and the insoluble solids were removed by filtration. The solid material was then subjected to vacuum to remove heptane. The product obtained was a white waxy material.

### Example 6

C₃₀₊ normal alpha olefin wax was purified by passing it over an activated alumina column. To a 20 mL glass vial containing a stirbar was add 5.0 g of the purified C₃₀₊ normal alpha olefin wax. The vial was then warmed to 74° C. To the warmed glass vial was added, with stirring, 2.0 g of 7 wt % Al MMAO-3A in heptanes. Immediately after, 0.5 mL of a 2 mg/mL toluene solution of Metallocene I was injected into the stirring solution and allowed to react for 2 hours. (Al:Zr equivalent ratio of 3,000.) The solution was then cooled to room temperature and dried under vacuum. GPC analysis indicated that the olefin wax oligomer within the olefin wax composition had a Mₙ of 15,290 g/mole, a M_{w} of 20,070 g/mole, and a polydispersity index of 1.31.

### Example 7

C_{30+HA} normal alpha olefin wax was purified by passing it over an activated alumina column. To a 20 mL glass vial containing a stirbar was add 5.0 g of the purified C_{30+HA} normal alpha olefin wax. The vial was then warmed to 74° C. To the warmed glass vial was added, with stirring, 2.0 g of 7 wt Al MMAO-3A in heptanes. Immediately after, 0.5 mL of a 2 mg/mL toluene solution of Metallocene I was injected into the stirring solution and allowed to react for 2 hours. (Al:Zr equivalent ratio of 3,000.) The solution was then cooled to room temperature and dried under vacuum. A GPC analysis of the olefin wax oligomer composition indicated that the olefin wax oligomer within the olefin wax composition had a Mₙ of 15,610 g/mole, a M_{w} of 20,500 g/mole, and a polydispersity index of 1.31.

What is claimed:

1. An oligomerization method, comprising:
a) contacting an olefin wax and a catalyst system, the catalyst system comprising
1) a metallocene, and
2) an activator;
and
b) forming an olefin wax oligomer composition under oligomerization conditions.

2. The oligomerization method of claim 1, wherein the activator comprises an alumoxane.

3. The oligomerization method of claim 1, wherein the activator comprises
a) a first activator comprising a chemically-treated solid oxide, and
b) a second activator comprising at least one of:
i) an organoaluminum compound having a formula Al(X¹⁰)ₙ(X¹¹)₃₋ₙ wherein X¹⁰ is independently a C₁ to C₂₀ hydrocarbyl, X¹¹ is independently a halide, a hydride, or a C₁ to C₂₀ hydrocarboxide, and n is a number from 1 to 3;
ii) an organozinc compound having the formula Zn X⁴⁰X⁴¹ wherein X⁴⁰ is independently a C₁ to C₂₀ hydrocarbyl and X⁴¹ is independently a halide, a hydride, or a C₁ to C₂₀ hydrocarbyl; and
iii) an organoboron compound having a formula B(X⁴²)ₙ(X⁴³)₃₋ₙ wherein X⁴² is independently a C₁ to C₂₀ hydrocarbyl, X⁴³ is independently a halide, a hydride, or a C₁ to C₂₀ hydrocarboxide, and n is a number from 1 to 3; and

4. The oligomerization method of claim 1, wherein
a) a first activator comprises a chemically-treated solid oxide;
b) a second activator comprising an organoaluminum compound;
c) an aluminum of the organoaluminum compound to metal of the metallocene molar ratio (Al:metal) is from 1:1 to 10,000:1;
d) a first activator to metallocene weight ratio is from 0.1:1 to 100,000:1; and
e) an olefin wax monomer to metallocene weight ratio is from 100:1 to 1,000,000,000:1.

5. The oligomerization method of claim 1, wherein the metallocene comprises a metallocene having a formula X²¹X²²X²³X²⁴M¹ wherein
M¹ is titanium, zirconium, hafnium, vanadium, niobium, tantalum, chromium, molybdenum, or tungsten;
X²¹ and X²² are substituted or unsubstituted pi-bonded η^{x≧5} ligands optionally connected by a linking group; and
X²³ and X²⁴ independently are a halide, a C₁ to C₂₀ hydrocarboxide, a C₁ to C₃₀ hydrocarbyl, or a C₃ to C₂₀ trihydrocarbylsiloxy.

6. The oligomerization method of claim 1, wherein the metallocene has a formula: 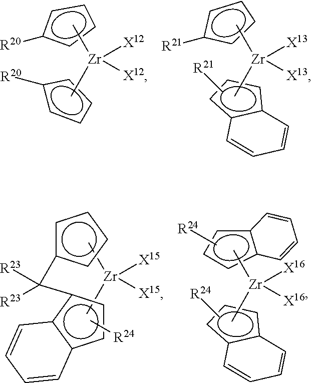
or any combination thereof, wherein
i) each R²⁰, R²¹, R²³, and R²⁴ is independently a hydrogen, a C₁ to C₂₀ alkyl group, or a C₃ to C₂₀ alkenyl group, and
ii) each X¹², X¹³, X¹⁵, and X¹⁶ is independently F, Cl, Br, or I.

7. The oligomerization method of claim 1, wherein the metallocene has a formula: 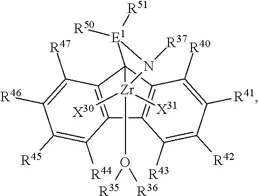
wherein
a) E¹ is C, Si, Ge, or Sn,
b) R⁴⁰, R⁴¹, R⁴², R⁴³, R⁴⁴, R⁴⁵, R⁴⁶, and R⁴⁷ are independently hydrogen or a C₁ to C₂₀ hydrocarbyl group,
c) optionally, R⁴¹ and R⁴² together form a ring and/or R⁴⁵ and R⁴⁶ together form a ring;
d) R⁵⁰ and R⁵¹ independently are hydrogen or a C₁-C₂₀ hydrocarbyl group;
e) R³⁵, R³⁶, and R³⁷ independently are a C₁-C₂₀ hydrocarbyl group; and
f) X³⁰ and X³¹ are independently halogen atoms.

8. The oligomerization method of claim 1, wherein the activator comprises fluorided alumina, chlorided alumina, sulfated alumina, fluorided silica-alumina, or any combination thereof.

9. The oligomerization method of claim 1, wherein the activator comprises fluorided silica-alumina.

10. The oligomerization method of claim 1, wherein the activator comprises a trialkylaluminum, an alkylaluminum sesquihalide, an alkylaluminum halide, a dialkyl zinc, a trialkylboron, a triaryl boron, or any combination thereof.

11. The oligomerization method of claim 1, wherein activator comprises a trialkylaluminum.

12. The oligomerization method of claim 1, wherein the olefin wax comprises:
a) at least 70 wt % olefins having from 20 to 24 carbon atoms,
b) at least 60 wt % olefins having from 24 to 28 carbon atoms;
c) at least 70 wt % olefins having from 26 to 28 carbon atoms; or
d) at least 70 wt % olefins having greater than 30 carbon atoms.

13. The oligomerization method of claim 1, wherein the olefin wax comprises:
a) i) at least 70 wt % olefins having from 20 to 24 carbon atoms, and ii) greater than 70 mole % alpha olefin;
b) i) at least 60 wt % olefins having from 24 to 28 carbon atoms, and ii) greater than 45 mole % alpha olefin;
c) i) at least 70 wt % olefins having from 26 to 28 carbon atoms, and ii) greater than 75 mole % alpha olefin; or
d) i) at least 70 wt % olefins having greater than 30 carbon atoms, and ii) greater than 45 mole % alpha olefin.

14. The oligomerization method of claim 1, wherein at least 60 weight % of the olefin wax is converted to olefin wax oligomer.

15. The oligomerization method of claim 1, wherein the olefin wax oligomer composition comprises olefin wax oligomer and olefin wax monomer and the olefin wax oligomer composition comprises from 50 to 95 weight percent olefin wax oligomers.

16. The oligomerization method of claim 1, wherein the olefin wax oligomer composition consists essentially of olefin wax oligomers and olefin wax monomer.

17. The oligomerization method of claim 1, wherein the olefin wax oligomer composition has a 25° C. needle penetration at least 15 percent lower than the needle penetration of the olefin wax.

18. The oligomerization method of claim 1, wherein the olefin wax oligomer composition has a drop melt point, in ° C., at least 15 percent higher than the olefin wax.

19. The oligomerization method of claim 15, wherein
A)
1) the olefin wax comprises a C₂₀ to C₂₄ alpha olefin;
2) the olefin wax oligomer composition comprises greater than 75 weight percent olefin wax oligomers; and
3) the olefin wax oligomer composition has
a) a 25° C. needle penetration at least 25 percent lower than the needle penetration of the olefin wax;
b) a drop melt point, in ° C., at least 15 percent higher than the olefin wax;
c) a 100° C. viscosity at least 40 percent higher than the olefin wax; and
d) a polydispersity index as measured by GPC ranging from 2.5 to 15.5;
B)
1) the olefin wax comprises a C₂₄ to C₂₈ alpha olefin;
2) the olefin wax oligomer composition comprises greater than 65 weight percent olefin wax oligomers; and
3) the olefin wax oligomer composition has
a) a 25° C. needle penetration at least 20 percent lower than the needle penetration of the olefin wax;
b) a drop melt point, in ° C., at least 20 percent higher than the olefin wax;
c) a 100° C. viscosity at least 60 percent higher than the olefin wax; and
d) a polydispersity index as measured by GPC ranging from 2.5 to 15.5;
C)
1) the olefin wax comprises a C₂₆ to C₂₈ alpha olefin;
2) the olefin wax oligomer composition comprises greater than 60 weight percent olefin wax oligomers; and
3) the olefin wax oligomer composition has
a) a 25° C. needle penetration at least 20 percent lower than the needle penetration of the olefin wax;
b) a drop melt point, in ° C., at least 25 percent higher than the olefin wax;
c) a 100° C. viscosity at least 60 percent higher than the olefin wax; and
d) a polydispersity index as measured by GPC ranging from 2.5 to 15.5; or
D)
1) the olefin wax comprises a C₃₀₊ alpha olefin;
2) the olefin wax oligomer composition comprises greater than 50 weight percent olefin wax oligomers; and
3) the olefin wax oligomer composition has
a) a 25° C. needle penetration at least 15 percent lower than the needle penetration of the olefin wax;
b) a drop melt point, in ° C., at least 30 percent higher than the olefin wax;
c) a 100° C. viscosity at least 80 percent higher than the olefin wax; and
d) a polydispersity index as measured by GPC ranging from 2.5 to 15.5.

20. The oligomerization method of claim 15, wherein the olefin wax oligomer having the greatest maximum peak height as measured by GPC has a molecular weight greater than 4,000 g/mole.

21. The oligomerization method of claims 15, wherein the olefin wax oligomer composition has a Mₙ as measured by GPC from 1,250 g/mole to 45,000 g/mole.

22. The oligomerization method of claim 15, wherein the olefin wax oligomer composition has a M_{w} as measured by GPC greater than 6,000 g/mole.

23. A method for producing an olefin wax oligomer composition, the method comprising:
a) contacting
1) an olefin wax comprising
i) (a) at least 70 wt % olefins having from 20 to 24 carbon atoms, and (b) greater than 70 mole % alpha olefin;
ii) (a) at least 60 wt % olefins having from 24 to 28 carbon atoms, and (b) greater than 45 mole % alpha olefin;
iii) (a) at least 70 wt % olefins having from 26 to 28 carbon atoms, and (b) greater than 75 mole % alpha olefin; or
iv) (a) at least 70 wt % olefins having greater than 30 carbon atoms, and (b) greater than 45 mole % alpha olefin; and
2) a catalyst system, the catalyst system comprising;
i) a metallocene having a formula: 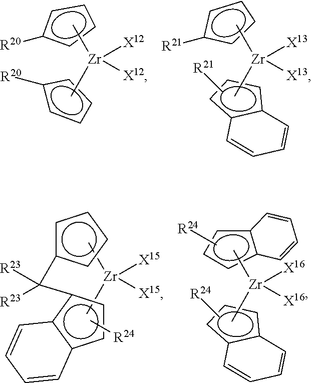
or any combination thereof, wherein
(a) each R²⁰, R²¹, R²³, and R²⁴ is independently a hydrogen, a C₁ to C₂₀ alkyl group, or a C₁ to C₂₀ alkenyl group, and
(b) each X¹², X¹³, X¹⁵, and X¹⁶ is independently F, Cl, Br, or I; or
a metallocene having the formula: 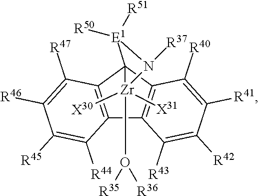
wherein
(a) E¹ is C, Si, Ge, or Sn,
(b) R⁴⁰, R⁴¹, R⁴², R⁴³, R⁴⁴, R⁴⁵, R⁴⁶, and R⁴⁷ are independently hydrogen or a C₁ to C₂₀ hydrocarbyl group,
(c) optionally, R⁴¹ and R⁴² together form a ring and/or R⁴⁵ and R⁴⁶ together form a ring;
(e) R⁵⁰ and R⁵¹ independently are hydrogen or a C₁-C₂₀ hydrocarbyl group;
(f) R³⁵, R³⁶, and R³⁷ independently are a C₁-C₂₀ hydrocarbyl group; and
(g) X³⁰ and X³¹ are independently Cl or Br; and
ii) an activator; and
b) forming an olefin wax oligomer composition under oligomerization conditions; wherein the olefin wax oligomer composition
i) comprises olefin wax oligomer and olefin wax monomer;
ii) comprises greater than 55 weight percent olefin wax oligomers;
iii) has a 25° C. needle penetration at least 25 percent lower than the needle penetration of the olefin wax; and
iv) has a 100° C. viscosity at least 40 percent higher than the olefin wax.

24. The method of claim 23, wherein the activator comprises an alumoxane.

25. The method of claim 23, wherein the activator comprises
a) a first activator comprising a chemically-treated solid oxide, wherein the chemically-treated solid oxide comprises fluorided alumina, chlorided alumina, sulfated alumina, fluorided silica-alumina, or any combination thereof, and
b) a second activator comprising a trialkylaluminum, an alkylaluminum sesquihalide, an alkylaluminum halide, or any combination thereof; and

26. The method of claim 25, wherein the olefin wax monomer and the catalyst system are contacted at
1) an aluminum of the organoaluminum compound to metal of the metallocene molar ratio is an Al:metal molar ratio ranging from 50:1 to 500:1,
2) a first activator to metallocene weight ratio ranging from 100:1 to 1,000:1,
3) an alpha olefin to metallocene weight ratio ranging from 1,000:1 to 100,000,000; and
4) an oligomerization temperature from 70° C. to 120° C.

27. The method of claim 23, wherein
A)
a) the olefin wax comprises the C₂₀ to C₂₄ alpha olefin;
b) the olefin wax oligomer composition comprises from 50 to 95 weight percent olefin wax oligomers; and
c) the olefin wax oligomer composition has
1) a 25° C. needle penetration at least 25 percent lower than the needle penetration of the olefin wax;
2) a drop melt point, in ° C., at least 15 percent higher than the olefin wax;
3) a 100° C. viscosity at least 40 percent higher than the olefin wax; and
4) has a Mₙ as measured by GPC greater than 1,000 g/mole, and
5) has a polydispersity index as measured by GPC of from 2.5 to 15.5;
B)
a) the olefin wax comprises the C₂₄ to C₂₈ alpha olefin;
b) the olefin wax oligomer composition comprises from 60 to 95 weight percent olefin wax oligomers; and
c) the olefin wax oligomer composition has
1) a 25° C. needle penetration at least 20 percent lower than the needle penetration of the olefin wax;
2) a drop melt point, in ° C., at least 20 percent higher than the olefin wax;
3) a 100° C. viscosity at least 60 percent higher than the olefin wax;
4) has a Mₙ as measured by GPC greater than 1,750 g/mole, and
5) has a polydispersity index as measured by GPC ranging from 2.5 to 15.5;
C)
a) the olefin wax comprises the C₂₆ to C₂₈ alpha olefin;
b) the olefin wax oligomer composition comprises from 60 to 95 weight percent olefin wax oligomers weight percent olefin wax oligomers; and
c) the olefin wax oligomer composition has
1) a 25° C. needle penetration at least 20 percent lower than the needle penetration of the olefin wax;
2) a drop melt point, in ° C., at least 25 percent higher than the olefin wax;
3) a 100° C. viscosity at least 60 percent higher than the olefin wax;
4) has a Mₙ as measured by GPC greater than 1,000 g/mole; and
5) has a polydispersity index as measured by GPC of from 2.5 to 15.5; or
D)
a) the olefin wax monomer comprises the C₃₀₊ alpha olefin;
b) the olefin wax oligomer composition comprises from 50 to 95 weight percent olefin wax oligomers;
c) the olefin wax composition has
1) a 25° C. needle penetration at least 15 percent lower than the needle penetration of the olefin wax;
2) a drop melt point, in ° C., at least 30 percent higher than the olefin wax;
3) a 100° C. viscosity at least 80 percent higher than the olefin wax;
4) has a Mₙ as measured by GPC greater than 1,000 g/mole, and
5) has a polydispersity index as measured by GPC of from 2.5 to 15.5.

28. An olefin wax oligomer composition prepared from olefin wax comprising olefin wax oligomers and olefin wax monomer, wherein the olefin wax oligomer composition has at least four (4) of the following properties:
1) greater than 50 weight percent olefin wax oligomers;
2) a 25° C. needle penetration at least 15 percent lower than the needle penetration of the starting olefin wax;
3) a drop melt point, in ° C., at least 15 percent higher than the starting olefin wax;
4) a 100° C. kinematic viscosity at least 40 percent higher than the starting olefin wax monomer;
5) a M_{w} as measured by GPC greater than 6,000 g/mole;
6) a Mₙ as measured by GPC greater than 1,000 g/mole; and
7) a polydispersity index as measured by GPC greater than 2.5.

29. The composition of claim 28, having at least five (5) of the listed properties.

30. The composition of claim 28, having at least six (6) of the listed properties.

31. The composition of claim 28, having all of the listed properties.

32. The composition of claim 28, having 55 to 95 weight percent olefin wax oligomers.

33. The composition of claim 28, wherein the olefin wax oligomer contains monomer units of an alpha olefin wax.

34. The composition of claim 28, wherein the olefin wax oligomer contains monomer units of an olefin wax selected from:
a) an olefin wax having at least 70 wt % olefins having from 20 to 24 carbon atoms,
b) an olefin wax having at least 60 wt % olefins having from 24 to 28 carbon atoms;
c) an olefin wax having at least 70 wt % olefins having from 26 to 28 carbon atoms; and
d) an olefin wax having at least 70 wt % olefins having greater than 30 carbon atoms.
